SEQUENCE LISTING (193 sequences)

SEQ ID NO: 1 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 2 - protein, 133 aa, synthetic construct [sequence omitted]

SEQ ID NO: 3 - protein, 133 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 4 - protein, 133 aa, synthetic construct
APTSSSTKKTQLQLEHLLLDLQMILNGINNYKNPKLTRMLTEKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 5 - protein, 119 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSS

SEQ ID NO: 6 - protein, 10 aa, synthetic construct
GSIFSINVMG

SEQ ID NO: 7 - protein, 16 aa, synthetic construct
AISSGGSTNYADSVKG

SEQ ID NO: 8 - protein, 11 aa, synthetic construct
ASSWYEDETDY

SEQ ID NO: 9 - protein, 120 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSA

SEQ ID NO: 10 - protein, 121 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSAS

SEQ ID NO: 11 - protein, 122 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSAST

SEQ ID NO: 12 - protein, 122 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSAAA

SEQ ID NO: 13 - protein, 121 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSAH

SEQ ID NO: 14 - protein, 121 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSGS

SEQ ID NO: 15 - protein, 121 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSPP

SEQ ID NO: 16 - protein, 122 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSPPP

SEQ ID NO: 17 - protein, 120 aa, synthetic construct
EVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSGP

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 21 - protein, 230 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGGSPG

SEQ ID NO: 22 - protein, 8 aa, synthetic construct
IHVTLKSL

SEQ ID NO: 23 - protein, 8 aa, synthetic construct
NSYTIKGL

SEQ ID NO: 24 - protein, 8 aa, synthetic construct
SNESLSLS

SEQ ID NO: 25 - protein, 8 aa, synthetic construct
QVSSSLSP

SEQ ID NO: 26 - protein, 8 aa, synthetic construct
PTSTSLSP

SEQ ID NO: 27 - protein, 8 aa, synthetic construct
ESLSLSEE

SEQ ID NO: 28 - protein, 8 aa, synthetic construct
ASLSLAPV

SEQ ID NO: 29 - protein, 8 aa, synthetic construct
SQESLSLS

SEQ ID NO: 30 - protein, 4 aa, synthetic construct
PLGL

SEQ ID NO: 31 - protein, 10 aa, synthetic construct
APAGLIVPYN

SEQ ID NO: 32 - protein, 8 aa, synthetic construct
PVSLRSGS

SEQ ID NO: 33 - protein, 9 aa, synthetic construct
PANLVAPDP

SEQ ID NO: 34 - protein, 8 aa, synthetic construct
MPYDLYHP

SEQ ID NO: 35 - protein, 10 aa, synthetic construct
ISSGLLSGRS

SEQ ID NO: 36 - protein, 8 aa, synthetic construct
RAAAVKSP

SEQ ID NO: 37 - protein, 8 aa, synthetic construct
RPLALWRS

SEQ ID NO: 38 - protein, 8 aa, synthetic construct
TQKPLGLS

SEQ ID NO: 39 - protein, 8 aa, synthetic construct
RSKYLATA

SEQ ID NO: 40 - protein, 8 aa, synthetic construct
GRPRHQGV

SEQ ID NO: 41 - protein, 4 aa, synthetic construct
GLFG

SEQ ID NO: 42 - protein, 4 aa, synthetic construct
GFLG

SEQ ID NO: 43 - protein, 7 aa, synthetic construct
AGRRAAK

SEQ ID NO: 44 - protein, 5 aa, synthetic construct
FRLWA

SEQ ID NO: 45 - protein, 5 aa, synthetic construct
FRLWS

SEQ ID NO: 46 - protein, 8 aa, synthetic construct
NFFGVGGE

SEQ ID NO: 47 - protein, 8 aa, synthetic construct
PMKRLTLA

SEQ ID NO: 48 - protein, 8 aa, synthetic construct
FPLATYAP

SEQ ID NO: 49 - protein, 8 aa, synthetic construct
FLVGGASL

SEQ ID NO: 50 - protein, 8 aa, synthetic construct
KPMQFLGD

SEQ ID NO: 51 - protein, 8 aa, synthetic construct
GIVRAKGV

SEQ ID NO: 52 - protein, 8 aa, synthetic construct
ALFKSSFP

SEQ ID NO: 53 - protein, 8 aa, synthetic construct
SGRRSPGG

SEQ ID NO: 54 - protein, 8 aa, synthetic construct
SLGRRPGG

SEQ ID NO: 55 - protein, 8 aa, synthetic construct
SLSGRRGG

SEQ ID NO: 56 - protein, 8 aa, synthetic construct
SLSLGRRG

SEQ ID NO: 57 - protein, 8 aa, synthetic construct
SLSLSGRR

SEQ ID NO: 58 - protein, 6 aa, synthetic construct
GGPRRL

SEQ ID NO: 59 - protein, 6 aa, synthetic construct
GGPLRL

SEQ ID NO: 60 - protein, 6 aa, synthetic construct
GGPKLL

SEQ ID NO: 61 - protein, 6 aa, synthetic construct
GGPRNL

SEQ ID NO: 62 - protein, 6 aa, synthetic construct
GGPRML

SEQ ID NO: 63 - protein, 8 aa, synthetic construct
EHLRSPGG

SEQ ID NO: 64 - protein, 8 aa, synthetic construct
FRSGVPGG

SEQ ID NO: 65 - protein, 8 aa, synthetic construct
SLLLRTGN

SEQ ID NO: 66 - protein, 8 aa, synthetic construct
AGLRSPGG

SEQ ID NO: 67 - protein, 8 aa, synthetic construct
SLFRSAGP

SEQ ID NO: 68 - protein, 8 aa, synthetic construct
SLFRAPGP

SEQ ID NO: 69 - protein, 8 aa, synthetic construct
WLFRSPLG

SEQ ID NO: 70 - protein, 8 aa, synthetic construct
SRLRSPQG

SEQ ID NO: 71 - protein, 8 aa, synthetic construct
SLVLSGRR

SEQ ID NO: 72 - protein, 8 aa, synthetic construct
KQLRHMRG

SEQ ID NO: 73 - protein, 8 aa, synthetic construct
LSGRSDNH

SEQ ID NO: 74 - protein, 4 aa, synthetic construct
LSGK

SEQ ID NO: 75 - protein, 4 aa, synthetic construct
LSGR

SEQ ID NO: 76 - protein, 8 aa, synthetic construct
RQARVVGG

SEQ ID NO: 77 - protein, 8 aa, synthetic construct
RQRRVVGG

SEQ ID NO: 78 - protein, 8 aa, synthetic construct
RQYRVVGG

SEQ ID NO: 79 - protein, 8 aa, synthetic construct
SKGRSLIG

SEQ ID NO: 80 - protein, 8 aa, synthetic construct
PRFKIIGG

SEQ ID NO: 81 - protein, 8 aa, synthetic construct
KQLRVVNG

SEQ ID NO: 82 - protein, 8 aa, synthetic construct
IQPRITGG

SEQ ID NO: 83 - protein, 8 aa, synthetic construct
KQSRKFVP

SEQ ID NO: 84 - protein, 8 aa, synthetic construct
GRQSRAGG

SEQ ID NO: 85 - protein, 8 aa, synthetic construct
SGRSSPGG

SEQ ID NO: 86 - protein, 8 aa, synthetic construct
SSGRSPGG

SEQ ID NO: 87 - protein, 8 aa, synthetic construct
SLSGRSGG

SEQ ID NO: 88 - protein, 8 aa, synthetic construct
SLSSGRSG

SEQ ID NO: 89 - protein, 8 aa, synthetic construct
KLSLSGRS

SEQ ID NO: 90 - protein, 7 aa, synthetic construct
PLRLSRA

SEQ ID NO: 91 - protein, 7 aa, synthetic construct
PLKLSRA

SEQ ID NO: 92 - protein, 8 aa, synthetic construct
PLGLSGRS

SEQ ID NO: 93 - protein, 8 aa, synthetic construct
PLGLRSRA

SEQ ID NO: 94 - protein, 8 aa, synthetic construct
PLGLKSRA

SEQ ID NO: 95 - protein, 6 aa, synthetic construct
RGSRAG

SEQ ID NO: 96 - protein, 6 aa, synthetic construct
RLSRGK

SEQ ID NO: 97 - protein, 6 aa, synthetic construct
RGSRGG

SEQ ID NO: 98 - protein, 6 aa, synthetic construct
KGSRAG

SEQ ID NO: 99 - protein, 6 aa, synthetic construct
KLSRGK

SEQ ID NO: 100 - protein, 6 aa, synthetic construct
GRSRAG

SEQ ID NO: 101 - protein, 6 aa, synthetic construct
LRSRGK

SEQ ID NO: 102 - protein, 6 aa, synthetic construct
GRSRGG

SEQ ID NO: 103 - protein, 6 aa, synthetic construct
GKSRAG

SEQ ID NO: 104 - protein, 6 aa, synthetic construct
LKSRGK

SEQ ID NO: 105 - protein, 9 aa, synthetic construct
VPLSLYSGP

SEQ ID NO: 106 - protein, 8 aa, synthetic construct
DSGGFMLT

SEQ ID NO: 107 - protein, 6 aa, synthetic construct
HEQLTV

SEQ ID NO: 108 - protein, 6 aa, synthetic construct
VPLSLY

SEQ ID NO: 109 - protein, 9 aa, synthetic construct
DLLAVVAAS

SEQ ID NO: 110 - protein, 10 aa, synthetic construct
GMPKDLYHAS

SEQ ID NO: 111 - protein, 10 aa, synthetic construct
IVGRPRHQGV

SEQ ID NO: 112 - protein, 9 aa, synthetic construct
MPYDLYHPS

SEQ ID NO: 113 - protein, 8 aa, synthetic construct
VPLSLYSG

SEQ ID NO: 114 - protein, 13 aa, synthetic construct
ISSGLLSGRSDQP

SEQ ID NO: 115 - skipped

SEQ ID NO: 116 - protein, 8 aa, synthetic construct
QQGNVFSC

SEQ ID NO: 117 - protein, 7 aa, synthetic construct
LSLSGRS

SEQ ID NO: 118 - protein, 20 aa, synthetic construct
GPPSGSSPMPYDLYHPSGGG

SEQ ID NO: 119 - protein, 21 aa, synthetic construct
GPPSGSSPGMPYDLYHPSGGG

SEQ ID NO: 120 - protein, 15 aa, synthetic construct
GGPMPYDLYHPSGGG

SEQ ID NO: 121 - protein, 15 aa, synthetic construct
GSPMPYDLYHPSGGG

SEQ ID NO: 122 - protein, 17 aa, synthetic construct
SSGGPMPYDLYHPSGGG

SEQ ID NO: 123 - protein, 18 aa, synthetic construct
SGSSPGMPYDLYHPSGGG

SEQ ID NO: 124 - protein, 19 aa, synthetic construct
GSSGPSGMPYDLYHPSGGG

SEQ ID NO: 125 - protein, 20 aa, synthetic construct
GSSSGPGSMPYDLYHPSGGG

SEQ ID NO: 126 - protein, 20 aa, synthetic construct
GPPSGSSPVPLSLYSGSGGG

SEQ ID NO: 127 - protein, 16 aa, synthetic construct
GGSPVPLSLYSGSGGG

SEQ ID NO: 128 - protein, 16 aa, synthetic construct
GSGGVPLSLYSGSGGG

SEQ ID NO: 129 - protein, 17 aa, synthetic construct
GGSPGVPLSLYSGSGGG

SEQ ID NO: 130 - protein, 18 aa, synthetic construct
GGSSGPVPLSLYSGSGGG

SEQ ID NO: 131 - protein, 19 aa, synthetic construct
GGSSGSGVPLSLYSGSGGG

SEQ ID NO: 132 - protein, 19 aa, synthetic construct
GGSPGSPVPLSLYSGSGGG

SEQ ID NO: 133 - protein, 26 aa, synthetic construct
GSPGVPLSLYSGSSPMPYDLYHPSGG

SEQ ID NO: 134 - protein, 15 aa, synthetic construct
GGSGGSVPLSLYSGP

SEQ ID NO: 135 - protein, 18 aa, synthetic construct
GGSGGSGGSVPLSLYSGP

SEQ ID NO: 136 - protein, 17 aa, synthetic construct
GGSGGSMPYDLYHPSGP

SEQ ID NO: 137 - protein, 20 aa, synthetic construct
GGSGGSGGSMPYDLYHPSGP

SEQ ID NO: 138 - protein, 17 aa, synthetic construct
GGSGGSDSGGFMLTSGP

SEQ ID NO: 139 - protein, 20 aa, synthetic construct
GGSGGSGGSDSGGFMLTSGP

SEQ ID NO: 140 - protein, 17 aa, synthetic construct
GGSGGSRAAAVKSPSGP

SEQ ID NO: 141 - protein, 20 aa, synthetic construct
GGSGGSGGSRAAAVKSPSGP

SEQ ID NO: 142 - protein, 19 aa, synthetic construct
GGSGGSISSGLLSGRSSGP

SEQ ID NO: 143 - protein, 22 aa, synthetic construct
GGSGGSGGSISSGLLSGRSSGP

SEQ ID NO: 144 - protein, 19 aa, synthetic construct
SPGGGGPMPYDLYHPSGGG

SEQ ID NO: 145 - protein, 6 aa, synthetic construct
GGSSPP

SEQ ID NO: 146 - protein, 4 aa, synthetic construct
GSGP

SEQ ID NO: 147 - protein, 4 aa, synthetic construct
GSPG

SEQ ID NO: 148 - protein, 4 aa, synthetic construct
GGSG

SEQ ID NO: 149 - protein, 9 aa, synthetic construct
GPPSGSSPG

SEQ ID NO: 150 - protein, 8 aa, synthetic construct
GPPSGSSP

SEQ ID NO: 151 - protein, 4 aa, synthetic construct
GGPS

SEQ ID NO: 152 - protein, 5 aa, synthetic construct
GSGPS

SEQ ID NO: 153 - protein, 6 aa, synthetic construct
GSSGGP

SEQ ID NO: 154 - skipped

SEQ ID NO: 155 - protein, 6 aa, synthetic construct
GSGSPS

SEQ ID NO: 156 - protein, 7 aa, synthetic construct
PSGSSPG

SEQ ID NO: 157 - protein, 5 aa, synthetic construct
SGSPS

SEQ ID NO: 158 - protein, 7 aa, synthetic construct
SPGGGGP

SEQ ID NO: 159 - skipped

SEQ ID NO: 160 - protein, 4 aa, synthetic construct
SGGG

SEQ ID NO: 161 - protein, 5 aa, synthetic construct
GSGGG

SEQ ID NO: 162 - protein, 5 aa, synthetic construct
PGSGS

SEQ ID NO: 163 - protein, 17 aa, synthetic construct
GGSSPPGGGSSGGGSGP

SEQ ID NO: 164 - protein, 5 aa, synthetic construct
GGGGS

SEQ ID NO: 165 - protein, 10 aa, synthetic construct
GGGGSGGGGS

SEQ ID NO: 166 - protein, 13 aa, synthetic construct
GGSGGGSGGGGGS

SEQ ID NO: 167 - protein, 18 aa, synthetic construct
GGSGGSGGSGGSGGSSGP

SEQ ID NO: 168 - protein, 6 aa, synthetic construct
PGGSGP

SEQ ID NO: 169 - protein, 120 aa, synthetic construct [sequence omitted]

SEQ ID NO: 170 - protein, 223 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 171 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 172 - protein, 8 aa, synthetic construct
GYTFTNYY

SEQ ID NO: 173 - protein, 8 aa, synthetic construct
INPSNGGT

SEQ ID NO: 174 - protein, 13 aa, synthetic construct
ARRDYRFDMGFDY

SEQ ID NO: 175 - protein, 119 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKTSENLYFQ

SEQ ID NO: 176 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 10 aa, synthetic construct
KGVSTSGYSY

SEQ ID NO: 178 - skipped

SEQ ID NO: 179 - protein, 9 aa, synthetic construct
QHSRDLPLT

SEQ ID NO: 180 - protein, 113 aa, synthetic construct [sequence omitted]

SEQ ID NO: 181 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 182 - protein, 8 aa, synthetic construct
GITFSNSG

SEQ ID NO: 183 - protein, 17 aa, synthetic construct
VIWYDGSKRYYADSVKG

SEQ ID NO: 184 - protein, 6 aa, synthetic construct
ATNDDY

SEQ ID NO: 185 - protein, 216 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 186 - protein, 107 aa, synthetic construct [sequence omitted]

SEQ ID NO: 187 - protein, 213 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 188 - protein, 6 aa, synthetic construct
QSVSSY

SEQ ID NO: 189 - skipped

SEQ ID NO: 190 - protein, 9 aa, synthetic construct
QQSSNWPRT

SEQ ID NO: 191 - protein, 599 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGGGSSPPGGGSSGGGSGPAPTSSSTKKTQLQLEHLLLDLQMILNGINNYKNPKLTRMLTEKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 192 - protein, 590 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSPGGGGPMPYDLYHPSGGGEVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSSAAA

SEQ ID NO: 193 - protein, 587 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSPGGGGPMPYDLYHPSGGGEVQLVESGGGLVQPGGSLRLSCAASGSIFSINVMGWYRQAPGKQRELVAAISSGGSTNYADSVKGRFTISRDNAKNTVYLQMNSLKPEDTAVYYCMYASSWYEDETDYWGQGTQVTVSS

(19) United States
(12) Patent Application Publication
(10) Pub. No.: US 2025/0101075 A1
(43) Pub. Date: Mar. 27, 2025

(54) MASKED IL-2 CYTOKINES AND METHODS OF USE THEREOF

(71) Applicant: Xilio Development, Inc., Waltham, MA (US)

(72) Inventors: Parker JOHNSON, Waltham, MA (US); Ertan ERYILMAZ, Waltham, MA (US); Dheeraj TOMAR, Waltham, MA (US); Benjamin NICHOLSON, Waltham, MA (US); Kurt JENKINS, Waltham, MA (US); Carl Uli BIALUCHA, Waltham, MA (US)

(21) Appl. No.: 18/821,234

(22) Filed: Aug. 30, 2024

Related U.S. Application Data
(60) Provisional application No. 63/579,684, filed on Aug. 30, 2023.

(51) Int. Cl. C07K 14/55; A61K 38/00; A61P 35/00; C07K 16/24

(52) U.S. Cl. CPC C07K 14/55; A61P 35/00; C07K 16/246; A61K 38/00; C07K 2317/52; C07K 2317/569; C07K 2317/73; C07K 2317/92; C07K 2319/30; C07K 2319/50

(57) ABSTRACT

The present invention provides, among other things, a masked cytokine comprising an attenuated interleukin 2 (IL-2) polypeptide, a VHH masking moiety, a carrier moiety and anti-PD1 targeting moiety. In some embodiments, the IL-2 polypeptide is engineered to be activatable by a protease at a target site, such as in a tumor microenvironment, by including a proteolytically cleavable linker. The VHH masking moiety blocks, occludes, inhibits (e.g., decreases) or otherwise prevents (e g masks) the activity or binding of the cytokine to its cognate receptor or protein. Upon proteolytic cleavage of the cleavable linker at the target site, the IL-2 polypeptide becomes activated, which renders it capable of binding to its cognate receptor or protein with increased affinity.

### CROSS-REFERENCE TO RELATED APPLICATIONS

This application claims priority to, and the benefit of, U.S. provisional application No. 63/579,684, filed Aug. 30, 2023, the contents of which is hereby incorporated by reference in its entirety.

### INCORPORATION-BY-REFERENCE OF SEQUENCE LISTING

The present specification refers to and includes a Sequence Listing filed herewith (submitted electronically as an XML file named XTX_IL2_04US1_SL.xml on Aug. 30, 2024). The .xml file was generated on Aug. 30, 2024, and is 171,326 bytes in size. The entire contents of the sequence listing are herein incorporated by reference in its entirety.

### BACKGROUND

Cancer is the second leading cause of death in the United States, accounting for more deaths than the next five leading causes (chronic respiratory disease, stroke, accidents, Alzheimer's disease, and diabetes). While great strides have been made especially with targeted therapies, there remains a great deal of work to do in this space. Immunotherapy and a branch of this field, immuno-oncology, is creating viable and exciting therapeutic options for treating malignancies. Specifically, it is now recognized that one hallmark of cancer is immune evasion and significant efforts have identified targets and developed therapies to these targets to reactivate the immune system to recognize and treat cancer.

Cytokine therapy is an effective strategy for stimulating the immune system to induce anti-tumor cytotoxicity. In particular, Proleukin (aldesleukin), a recombinant form of interleukin-2 (IL-2), has been approved by the FDA for the treatment of metastatic renal cell carcinoma and melanoma. Unfortunately, cytokines that are administered to patients generally have a very short half-life, thereby requiring frequent dosing. For instance, the product label of aldesleukin, marketed under the brand name Proleukin, states that the drug was shown to have a half-life of 85 minutes in patients who received a 5-minute intravenous (IV) infusion. In addition, administration of high doses of cytokine can cause adverse health outcomes, such as vascular leakage, through systemic immune activation. These findings illustrate the need for developing IL-2 polypeptide therapeutics that effectively target tumor without the side effects associated with systemic immune activation.

### SUMMARY OF THE INVENTION

The present invention provides, among other things a masked cytokine comprising an attenuated IL-2 and a VHH masking moiety. A VHH masking moiety binds to the IL-2 cytokine and inhibits a biological activity of the cytokine in undesired targets. Upon cleavage in desired targets (e.g., tumor), the masking moiety is released from the cytokine, activating the function of the IL-2 polypeptide. In particular, the masked cytokine comprising an attenuated IL-2 and VHH masking moieties of the present invention is characterized with (1) effective masking efficiency such that IL-2 cytokine's function is inhibited in undesired targets; (2) efficient IL-2 activation by protease to release the VHH masking moiety; (3) selective IL-2 activation in tumor, and not in plasma; and (4) in vivo efficacy (e.g., high tumor growth inhibition).

In one aspect, the present invention provides, among other things, a masked cytokine comprising an attenuated IL-2 polypeptide, a masking moiety comprising a heavy-chain only antibody (VHH), a carrier moiety, and an anti-PD1 targeting moiety.

In some embodiments, the attenuated IL-2 polypeptide comprises mutation at amino acid position R38 compared to the sequence of a mature IL-2 having SEQ ID NO: 2. In some embodiments, the attenuated IL-2 polypeptide comprises mutation at amino acid position F42 compared to the sequence of a mature IL-2 having SEQ ID NO: 2. In some embodiments, the attenuated IL-2 polypeptide comprises mutation at amino acid position K43 compared to the sequence of a mature IL-2 having SEQ ID NO: 2. In some embodiments, the attenuated IL-2 polypeptide comprises mutation at amino acid position Y45 compared to the sequence of a mature IL-2 having SEQ ID NO: 2. In some embodiments, the attenuated IL-2 polypeptide comprises mutation at amino acid position E62 compared to the sequence of a mature IL-2 having SEQ ID NO: 2. In some embodiments, the attenuated IL-2 polypeptide comprises mutation at amino acid position L72 compared to the sequence of a mature IL-2 having SEQ ID NO: 2. In some embodiments, the attenuated IL-2 polypeptide comprises mutation at amino acid position C125 compared to the sequence of a mature IL-2 having SEQ ID NO: 2.

In some embodiments, the attenuated IL-2 polypeptide comprises F42E and C125A mutations. In some embodiments, the attenuated IL-2 polypeptide comprises E62S and C125A mutations. In some embodiments, the attenuated IL-2 polypeptide comprises F42A and C125A mutations. In some embodiments, the attenuated IL-2 polypeptide comprises Y45R and C125A mutations. In some embodiments, the attenuated IL-2 polypeptide comprises F42S, E62S, and C125A mutations. In some embodiments, the attenuated IL-2 polypeptide comprises F42K mutation. In some embodiments, the attenuated IL-2 polypeptide comprises F42A, Y45A, L72G, and C125A mutations. In some embodiments, the attenuated IL-2 polypeptide comprises E62R mutation. In some embodiments, the attenuated IL-2 polypeptide comprises Y45N mutation. In some embodiments, the attenuated IL-2 polypeptide comprises K43A mutation. In some embodiments, the attenuated IL-2 polypeptide comprises Y45A and E62S mutations. In some embodiments, the attenuated IL-2 polypeptide comprises R38G, Y45A and E62S mutations. In some embodiments, the attenuated IL-2 polypeptide comprises C125A mutation.

In some embodiments, the attenuated IL-2 polypeptide comprises SEQ ID NO: 4.

In some embodiments, the VHH specifically binds to the IL-2 polypeptide.

In some embodiments, wherein the VHH comprises an HCDR1 comprising an amino acid sequence GSIFSINVMG (SEQ ID NO: 6), an HCDR2 comprising an amino acid sequence AISSGGSTNYADSVKG (SEQ ID NO: 7), and an HCDR3 comprising an amino acid sequence selected from ASSWYEDETDY (SEQ ID NO: 8).

In some embodiments, the VHH comprises an amino acid sequence that is at least 85% identical to SEQ ID NO: 5. In some embodiments, the VHH comprises an amino acid sequence that is at least 88% identical to SEQ ID NO: 5. In some embodiments, the VHH comprises an amino acid sequence that is at least 90% identical to SEQ ID NO: 5. In some embodiments, the VHH comprises an amino acid sequence that is at least 92% identical to SEQ ID NO: 5. In some embodiments, the VHH comprises an amino acid sequence that is at least 93% identical to SEQ ID NO: 5. In some embodiments, the VHH comprises an amino acid sequence that is at least 94% identical to SEQ ID NO: 5. In some embodiments, the VHH comprises an amino acid sequence that is at least 95% identical to SEQ ID NO: 5. In some embodiments, the VHH comprises an amino acid sequence that is at least 96% identical to SEQ ID NO: 5. In some embodiments, the VHH comprises an amino acid sequence that is at least 97% identical to SEQ ID NO: 5. In some embodiments, the VHH comprises an amino acid sequence that is at least 98% identical to SEQ ID NO: 5. In some embodiments, the VHH comprises an amino acid sequence that is at least 99% identical to SEQ ID NO: 5.

In some embodiments, the VHH comprises an amino acid sequence of SEQ ID NO: 5.

In some embodiments, the VHH comprises an amino acid extension. In some embodiments, the extension is at the N-terminus of the VHH. In some embodiments, the extension is at the C-terminus of the VHH.

In some embodiments, the amino extension comprises 1 amino acid. In some embodiments, the amino extension comprises 2 amino acids. In some embodiments, the amino extension comprises 3 amino acids. In some embodiments, the amino extension comprises 4 amino acids. In some embodiments, the amino extension comprises 5 amino acids. In some embodiments, the amino extension comprises 6 amino acids. In some embodiments, the amino extension comprises 7 amino acids. In some embodiments, the amino extension comprises 8 amino acids. In some embodiments, the amino extension comprises 9 amino acids. In some embodiments, the amino extension comprises 10 amino acids.

In some embodiments, the amino acid extension is residue A. In some embodiments, the amino acid extension is residues AS. In some embodiments, the amino acid extension is residues AST. In some embodiments, the amino acid extension is residues AAA. In some embodiments, the amino acid extension is residues AH. In some embodiments, the amino acid extension is residues GS. In some embodiments, the amino acid extension is residues PP. In some embodiments, the amino acid extension is residues PPP. In some embodiments, the amino acid extension is residues GP.

In some embodiments, the VHH comprises an amino acid sequence that is at least 85% identical to SEQ ID NO: 12. In some embodiments, the VHH comprises an amino acid sequence that is at least 88% identical to SEQ ID NO: 12. In some embodiments, the VHH comprises an amino acid sequence that is at least 90% identical to SEQ ID NO: 12. In some embodiments, the VHH comprises an amino acid sequence that is at least 92% identical to SEQ ID NO: 12. In some embodiments, the VHH comprises an amino acid sequence that is at least 93% identical to SEQ ID NO: 12. In some embodiments, the VHH comprises an amino acid sequence that is at least 94% identical to SEQ ID NO: 12. In some embodiments, the VHH comprises an amino acid sequence that is at least 95% identical to SEQ ID NO: 12. In some embodiments, the VHH comprises an amino acid sequence that is at least 96% identical to SEQ ID NO: 12. In some embodiments, the VHH comprises an amino acid sequence that is at least 97% identical to SEQ ID NO: 12. In some embodiments, the VHH comprises an amino acid sequence that is at least 98% identical to SEQ ID NO: 12. In some embodiments, the VHH comprises an amino acid sequence that is at least 99% identical to SEQ ID NO: 12.

In some embodiments, the VHH comprises an amino acid sequence of SEQ ID NO: 12.

In some embodiments, the targeting moiety comprises an antigen binding domain. In some embodiments the targeting moiety comprises Fab. In some embodiments the targeting moiety comprises a single chain Fv (scFv). In some embodiments the targeting moiety comprises a single domain antibody (VHH). In some embodiments the targeting moiety comprises one or more CDRs. In some embodiments the targeting moiety comprises a variable heavy chain (VH). In some embodiments the targeting moiety comprises a variable light chain (VL). In some embodiments the targeting moiety comprises a Fab-like bispecific antibodies (bsFab). In some embodiments the targeting moiety comprises a single-domain antibody-linked Fab (s-Fab). In some embodiments the targeting moiety comprises an antibody. In some embodiments the targeting moiety comprises a combination of antigen binding fragments.

In some embodiments, the targeting moiety comprises a heavy chain CDR1 sequence of GYTFTNYY (SEQ ID NO: 172), a heavy chain CDR2 sequence of INPSNGGT (SEQ ID NO: 173), and a heavy chain CDR3 sequence of ARRDYRFDMGFDY (SEQ ID NO: 174). In some embodiments, the targeting moiety comprises a light chain CDR1 sequence of KGVSTSGYSY (SEQ ID NO: 177), a light chain CDR2 sequence of LAS (SEQ ID NO: 178), and a light chain CDR3 sequence of QHSRDLPLT (SEQ ID NO: 179).

In some embodiments, the targeting moiety comprises a heavy chain CDR1 sequence of GYTFTNYY (SEQ ID NO: 172), a heavy chain CDR2 sequence of INPSNGGT (SEQ ID NO: 173), a heavy chain CDR3 sequence of ARRDYRFDMGFDY (SEQ ID NO: 174), a light chain CDR1 sequence of KGVSTSGYSY (SEQ ID NO: 177), a light chain CDR2 sequence of LAS (SEQ ID NO: 178), and a light chain CDR3 sequence of QHSRDLPLT (SEQ ID NO: 179).

In some embodiments, the targeting moiety comprises a heavy chain variable region at least about 80% identity to SEQ ID NO: 169. In some embodiments, the targeting moiety comprises a heavy chain variable region at least about 82% identity to SEQ ID NO: 169. In some embodiments, the targeting moiety comprises a heavy chain variable region at least about 85% identity to SEQ ID NO: 169. In some embodiments, the targeting moiety comprises a heavy chain variable region at least about 88% identity to SEQ ID NO: 169. In some embodiments, the targeting moiety comprises a heavy chain variable region at least about 89% identity to SEQ ID NO: 169. In some embodiments, the targeting moiety comprises a heavy chain variable region at least about 90% identity to SEQ ID NO: 169. In some embodiments, the targeting moiety comprises a heavy chain variable region at least about 91% identity to SEQ ID NO: 169. In some embodiments, the targeting moiety comprises a heavy chain variable region at least about 92% identity to SEQ ID NO: 169. In some embodiments, the targeting moiety comprises a heavy chain variable region at least about 93% identity to SEQ ID NO: 169. In some embodiments, the targeting moiety comprises a heavy chain variable region at least about 94% identity to SEQ ID NO: 169. In some embodiments, the targeting moiety comprises a heavy chain variable region at least about 95% identity to SEQ ID NO: 169. In some embodiments, the targeting moiety comprises a heavy chain variable region at least about 96% identity to SEQ ID NO: 169. In some embodiments, the targeting moiety comprises a heavy chain variable region at least about 97% identity to SEQ ID NO: 169. In some embodiments, the targeting moiety comprises a heavy chain variable region at least about 98% identity to SEQ ID NO: 169. In some embodiments, the targeting moiety comprises a heavy chain variable region at least about 99% identity to SEQ ID NO: 169.

In some embodiments, the targeting moiety comprises a light chain variable region at least about 80% identity to SEQ ID NO: 175. In some embodiments, the targeting moiety comprises a light chain variable region at least about 82% identity to SEQ ID NO: 175. In some embodiments, the targeting moiety comprises a light chain variable region at least about 85% identity to SEQ ID NO: 175. In some embodiments, the targeting moiety comprises a light chain variable region at least about 88% identity to SEQ ID NO: 175. In some embodiments, the targeting moiety comprises a light chain variable region at least about 89% identity to SEQ ID NO: 175. In some embodiments, the targeting moiety comprises a light chain variable region at least about 90% identity to SEQ ID NO: 175. In some embodiments, the targeting moiety comprises a light chain variable region at least about 91% identity to SEQ ID NO: 175. In some embodiments, the targeting moiety comprises a light chain variable region at least about 92% identity to SEQ ID NO: 175. In some embodiments, the targeting moiety comprises a light chain variable region at least about 93% identity to SEQ ID NO: 175. In some embodiments, the targeting moiety comprises a light chain variable region at least about 94% identity to SEQ ID NO: 175. In some embodiments, the targeting moiety comprises a light chain variable region at least about 95% identity to SEQ ID NO: 175. In some embodiments, the targeting moiety comprises a light chain variable region at least about 96% identity to SEQ ID NO: 175. In some embodiments, the targeting moiety comprises a light chain variable region at least about 97% identity to SEQ ID NO: 175. In some embodiments, the targeting moiety comprises a light chain variable region at least about 98% identity to SEQ ID NO: 175. In some embodiments, the targeting moiety comprises a light chain variable region at least about 99% identity to SEQ ID NO: 175.

In some embodiments, the targeting moiety comprises a heavy chain region at least about 80% identity to SEQ ID NO: 170. In some embodiments, the targeting moiety comprises a heavy chain region at least about 82% identity to SEQ ID NO: 170. In some embodiments, the targeting moiety comprises a heavy chain region at least about 85% identity to SEQ ID NO: 170. In some embodiments, the targeting moiety comprises a heavy chain region at least about 88% identity to SEQ ID NO: 170. In some embodiments, the targeting moiety comprises a heavy chain region at least about 89% identity to SEQ ID NO: 170. In some embodiments, the targeting moiety comprises a heavy chain region at least about 90% identity to SEQ ID NO: 170. In some embodiments, the targeting moiety comprises a heavy chain region at least about 91% identity to SEQ ID NO: 170. In some embodiments, the targeting moiety comprises a heavy chain region at least about 92% identity to SEQ ID NO: 170. In some embodiments, the targeting moiety comprises a heavy chain region at least about 93% identity to SEQ ID NO: 170. In some embodiments, the targeting moiety comprises a heavy chain region at least about 94% identity to SEQ ID NO: 170. In some embodiments, the targeting moiety comprises a heavy chain region at least about 95% identity to SEQ ID NO: 170. In some embodiments, the targeting moiety comprises a heavy chain region at least about 96% identity to SEQ ID NO: 170. In some embodiments, the targeting moiety comprises a heavy chain region at least about 97% identity to SEQ ID NO: 170. In some embodiments, the targeting moiety comprises a heavy chain region at least about 98% identity to SEQ ID NO: 170. In some embodiments, the targeting moiety comprises a heavy chain region at least about 99% identity to SEQ ID NO: 170.

In some embodiments, the targeting moiety comprises a light chain region at least about 80% identity to SEQ ID NO: 176. In some embodiments, the targeting moiety comprises a light chain region at least about 82% identity to SEQ ID NO: 176. In some embodiments, the targeting moiety comprises a light chain region at least about 85% identity to SEQ ID NO: 176. In some embodiments, the targeting moiety comprises a light chain region at least about 88% identity to SEQ ID NO: 176. In some embodiments, the targeting moiety comprises a light chain region at least about 89% identity to SEQ ID NO: 176. In some embodiments, the targeting moiety comprises a light chain region at least about 90% identity to SEQ ID NO: 176. In some embodiments, the targeting moiety comprises a light chain region at least about 91% identity to SEQ ID NO: 176. In some embodiments, the targeting moiety comprises a light chain region at least about 92% identity to SEQ ID NO: 176. In some embodiments, the targeting moiety comprises a light chain region at least about 93% identity to SEQ ID NO: 176. In some embodiments, the targeting moiety comprises a light chain region at least about 94% identity to SEQ ID NO: 176. In some embodiments, the targeting moiety comprises a light chain region at least about 95% identity to SEQ ID NO: 176. In some embodiments, the targeting moiety comprises a light chain region at least about 96% identity to SEQ ID NO: 176. In some embodiments, the targeting moiety comprises a light chain region at least about 97% identity to SEQ ID NO: 176. In some embodiments, the targeting moiety comprises a light chain region at least about 98% identity to SEQ ID NO: 176. In some embodiments, the targeting moiety comprises a light chain region at least about 99% identity to SEQ ID NO: 176.

In some embodiments, the targeting moiety comprises a heavy chain variable region of SEQ ID NO: 169, and a light chain variable region of SEQ ID NO: 175. In some embodiments, the targeting moiety comprises a heavy chain region of SEQ ID NO: 170, and a light chain region of SEQ ID NO: 176.

In some embodiments, the carrier moiety comprises a PEG molecule. In some embodiments, the carrier moiety comprises an albumin. In some embodiments, the carrier moiety comprises an albumin fragment. In some embodiments, the carrier moiety comprises an antibody Fc domain. In some embodiments, the carrier moiety comprises an antibody. In some embodiments, the carrier moiety comprises an antigen-binding fragment.

In some embodiments, an Fc domain comprises a first Fc polypeptide and a second Fc polypeptide.

In some embodiments, the first Fc polypeptide is linked to IL-2 polypeptide through a first linker and the second Fc polypeptide is linked to masking moiety through a second linker, and wherein the first or the second linker comprises a tumor-associated protease cleavage site such that the masking moiety is released from the IL-2 polypeptide.

In some embodiments, the first Fc polypeptide is linked to masking moiety through a first linker and the second Fc polypeptide is linked to IL-2 polypeptide through a second linker, and wherein the first or the second linker comprises a tumor-associated protease cleavage site such that the masking moiety is released from the IL-2 polypeptide.

In some embodiments, the first linker comprises a tumor-associated protease cleavage site. In some embodiments, the second linker comprises a tumor-associated protease cleavage site. In some embodiments, the first Fc polypeptide comprises Y349C, T366S, L368A, and Y407V, mutations and the second Fc polypeptide comprises S354C and T366W mutations. In some embodiments, the second Fc polypeptide comprises Y349C, T366S, L368A, and Y407V, and mutations and the first Fc polypeptide comprises S354C, and T366W mutations.

In some embodiments, the first Fc polypeptide comprises a CH3 domain comprising a modification that reduces or eliminates binding to Protein A, and the second Fc polypeptide comprises a CH3 domain that binds to Protein A. In some embodiments, the second Fc polypeptide comprises a CH3 domain comprising a modification that reduces or eliminates binding to Protein A, and the first Fc polypeptide comprises a CH3 domain that binds to Protein A.

In some embodiments, the modification that reduces or eliminates binding to Protein A is H435R and Y436F according to Kabat numbering.

In some embodiments, the first Fc polypeptide comprises a N297A mutation. In some embodiments, the second Fc polypeptide comprises a N297A mutation. In some embodiments, the first Fc polypeptide and second Fc polypeptide each comprises N297A mutation.

In some embodiments, the first Fc polypeptide comprises a IgG1 Fc domain or a fragment thereof. In some embodiments, the first Fc polypeptide comprises a IgG2 Fc domain or a fragment thereof. In some embodiments, the first Fc polypeptide comprises a IgG4 Fc domain or a fragment thereof. In some embodiments, the second Fc polypeptide comprises a IgG1 Fc domain or a fragment thereof. In some embodiments, the second Fc polypeptide comprises a IgG2 Fc domain or a fragment thereof. In some embodiments, the second Fc polypeptide comprises a IgG4 Fc domain or a fragment thereof.

In some embodiments, the first Fc polypeptide comprises I253A mutation. In some embodiments, the second Fc polypeptide comprises I253A mutation. In some embodiments, the first Fc polypeptide and second Fc polypeptide each comprises I253A mutation.

In some embodiments, the first Fc polypeptide comprises S354C, T366W, N297A, H435R, and Y435F mutations, and the second Fc polypeptide comprises Y349C; T366S; L368A; Y407V, and N297A mutations.

In some embodiments, the tumor-associated protease cleavage site comprises a sequence selected from Table 3. the tumor-associated protease cleavage site comprises MPYDLYHP (SEQ ID NO: 34).

In one aspect, the present invention provides, among other things, a masked cytokine comprising (a) an attenuated interleukin 2 (IL-2) polypeptide comprising an amino acid substitution of F42E and C125A; (b) a VHH masking moiety comprising a CDR1 of SEQ ID NO: 6, a CDR2 of SEQ ID NO: 7, and a CDR3 of SEQ ID NO: 8; (c) an Fc domain comprising a first Fc polypeptide linked to the attenuated IL-2 polypeptide through a first linker and a second Fc polypeptide linked to the VHH masking moiety through a second linker; and (d) an anti-PD1 targeting moiety.

In one aspect, the present invention provides, among other things, a masked cytokine comprising (a) an attenuated interleukin 2 (IL-2) polypeptide comprising SEQ ID NO: 4; (b) a VHH masking moiety comprising SEQ ID NO: 5 or SEQ ID NO: 12; (c) an Fc domain comprising a first Fc polypeptide of SEQ ID NO: 21 linked to the attenuated IL-2 polypeptide through a first linker and a second Fc polypeptide of SEQ ID NO: 20 linked to the VHH masking moiety through a second linker; and (d) an anti-PD1 targeting domain moiety comprising SEQ IDs: 169 and 175.

In one aspect, the present invention provides, among other things, a masked cytokine comprising three polypeptides: (a) a first polypeptide comprising a variable heavy chain of an anti-PD1 targeting moiety, a first Fc polypeptide, a non-cleavable linker, and an attenuated IL-2 polypeptide, (b) a second polypeptide comprising a variable heavy chain of an anti-PD1 targeting moiety, a second Fc polypeptide, a cleavable linker, and a VHH masking moiety, and (c) a third polypeptide comprising a variable light chain of an anti-PD1 targeting moiety.

In some embodiments, the first Fc polypeptide comprises SEQ ID NO: 21, and the second Fc polypeptide comprises SEQ ID NO: 20.

In some embodiments, the variable heavy chain of the anti-PD-1 targeting moiety comprises a hCDR1 of SEQ ID NO: 172, a hCDR2 of SEQ ID NO: 173, a hCDR3 of SEQ ID NO: 174. In some embodiments, the variable light chain of the anti-PD1 targeting moiety comprises a lCDR1 of SEQ ID NO: 177, a lCDR2 of SEQ ID NO: 178, a lCDR3 of SEQ ID NO: 178. In some embodiments, the variable heavy chain of the anti-PD-1 targeting moiety comprises a hCDR1 of SEQ ID NO: 172, a hCDR2 of SEQ ID NO: 173, a hCDR3 of SEQ ID NO: 174, and the variable light chain of the anti-PD1 targeting moiety comprises a lCDR1 of SEQ ID NO: 177, a lCDR2 of SEQ ID NO: 178, a lCDR3 of SEQ ID NO: 179.

In some embodiments, the variable heavy chain of the anti-PD-1 targeting moiety comprises SEQ ID NO: 169. In some embodiments, the variable light chain of the anti-PD1 targeting moiety comprises SEQ ID NO: 175. In some embodiments, the variable heavy chain of the anti-PD-1 targeting moiety comprises SEQ ID NO: 169, and the variable light chain of the anti-PD1 targeting moiety comprises SEQ ID NO: 175.

In some embodiments, the cleavable linker comprises MPYDLYHP (SEQ ID NO: 34). In some embodiments, the cleavable linker comprises SPGGGGPMPYDLYHPSGGG (SEQ ID NO: 144).

In some embodiments, the non-cleavable linker comprises GGSSPPGGGSSGGGSGP (SEQ ID NO: 163).

In one aspect, the present invention provides, among other things, a masked cytokine comprising three polypeptides: (a) a first polypeptide comprising (i) a variable heavy chain of an anti-PD1 targeting moiety comprising SEQ ID NO: 169, (ii) a first Fc polypeptide of SEQ ID NO: 21, (iii) a non-cleavable linker, and (iv) an attenuated IL-2 polypeptide of SEQ ID NO: 4, (b) a second polypeptide comprising (i) a variable heavy chain of an anti-PD1 targeting moiety comprising SEQ ID NO: 169, (ii) a second Fc polypeptide of SEQ ID NO: 20, (iii) a cleavable linker comprising MPYDLYHP (SEQ ID NO: 34), and (iv) a VHH masking moiety of SEQ ID NO: 5 or SEQ ID NO: 12, and (c) a third polypeptide comprising a variable light chain of an anti-PD1 targeting moiety comprising SEQ ID NO: 175.

In some embodiments, the first polypeptide comprises SEQ ID NO: 191. In some embodiments, the second polypeptide comprises SEQ ID NO: 192. In some embodiments, the third polypeptide comprises SEQ ID NO: 176. In some embodiments, the first polypeptide comprises SEQ ID NO: 191, the second polypeptide comprises SEQ ID NO: 192; and the third polypeptide comprises SEQ ID NO: 176.

In one aspect, the present invention provides, among other things, a masked cytokine comprising three polypeptides: (a) a first polypeptide comprising an amino sequence that is at least 90%, at least 95%, at least 96%, at least 97%, at least 98%, or at least 99% identical to SEQ ID NO: 191, (b) a second polypeptide comprising an amino sequence that is at least 90%, at least 95%, at least 96%, at least 97%, at least 98%, or at least 99% identical to SEQ ID NO: 193, and (c) a third polypeptide comprising an amino sequence that is at least 90%, at least 95%, at least 96%, at least 97%, at least 98%, or at least 99% identical to SEQ ID NO: 176.

In some embodiments, the first polypeptide comprises an amino acid sequence that is at least 90% identical to SEQ ID NO: 191. In some embodiments, the first polypeptide comprises an amino acid sequence that is at least 92% identical to SEQ ID NO: 191. In some embodiments, the first polypeptide comprises an amino acid sequence that is at least 95% identical to SEQ ID NO: 191. In some embodiments, the first polypeptide comprises an amino acid sequence that is at least 96% identical to SEQ ID NO: 191. In some embodiments, the first polypeptide comprises an amino acid sequence that is at least 97% identical to SEQ ID NO: 191. In some embodiments, the first polypeptide comprises an amino acid sequence that is at least 98% identical to SEQ ID NO: 191. In some embodiments, the first polypeptide comprises an amino acid sequence that is at least 99% identical to SEQ ID NO: 191.

In some embodiments, the second polypeptide comprises an amino acid sequence that is at least 90% identical to SEQ ID NO: 193. In some embodiments, the second polypeptide comprises an amino acid sequence that is at least 92% identical to SEQ ID NO: 193. In some embodiments, the second polypeptide comprises an amino acid sequence that is at least 95% identical to SEQ ID NO: 193. In some embodiments, the second polypeptide comprises an amino acid sequence that is at least 96% identical to SEQ ID NO: 193. In some embodiments, the second polypeptide comprises an amino acid sequence that is at least 97% identical to SEQ ID NO: 193. In some embodiments, the second polypeptide comprises an amino acid sequence that is at least 98% identical to SEQ ID NO: 193. In some embodiments, the second polypeptide comprises an amino acid sequence that is at least 99% identical to SEQ ID NO: 193.

In some embodiments, the third polypeptide comprises an amino acid sequence that is at least 90% identical to SEQ ID NO: 176. In some embodiments, the third polypeptide comprises an amino acid sequence that is at least 92% identical to SEQ ID NO: 176. In some embodiments, the third polypeptide comprises an amino acid sequence that is at least 95% identical to SEQ ID NO: 176. In some embodiments, the third polypeptide comprises an amino acid sequence that is at least 96% identical to SEQ ID NO: 176. In some embodiments, the third polypeptide comprises an amino acid sequence that is at least 97% identical to SEQ ID NO: 176. In some embodiments, the third polypeptide comprises an amino acid sequence that is at least 98% identical to SEQ ID NO: 176. In some embodiments, the third polypeptide comprises an amino acid sequence that is at least 99% identical to SEQ ID NO: 176.

In one aspect, the present invention provides, among other things, a masked cytokine comprising three polypeptides: (a) a first polypeptide comprising an amino sequence of SEQ ID NO: 191, (b) a second polypeptide comprising an amino sequence of SEQ ID NO: 193, and (c) a third polypeptide comprising an amino sequence of SEQ ID NO: 176.

In some embodiments, the masked cytokine has an EC50 of greater than the EC50 of an unmasked cytokine in a CD8 T cell pSTAT5 activity assay. In some embodiments, the masked cytokine has an EC50 of greater than the EC50 of an unmasked cytokine in a CD8 T cell pSTAT5 activity assay by at least 50-fold. In some embodiments, the masked cytokine has an EC50 of greater than the EC50 of an unmasked cytokine in a CD8 T cell pSTAT5 activity assay by at least 100-fold. In some embodiments, the masked cytokine has an EC50 of greater than the EC50 of an unmasked cytokine in a CD8 T cell pSTAT5 activity assay by at least 120-fold. In some embodiments, the masked cytokine has an EC50 of greater than the EC50 of an unmasked cytokine in a CD8 T cell pSTAT5 activity assay by at least 150-fold. In some embodiments, the masked cytokine has an EC50 of greater than the EC50 of an unmasked cytokine in a CD8 T cell pSTAT5 activity assay by at least 200-fold. In some embodiments, the masked cytokine has an EC50 of greater than the EC50 of an unmasked cytokine in a CD8 T cell pSTAT5 activity assay by at least 250-fold. In some embodiments, the masked cytokine has an EC50 of greater than the EC50 of an unmasked cytokine in a CD8 T cell pSTAT5 activity assay by at least 280-fold. In some embodiments, the masked cytokine has an EC50 of greater than the EC50 of an unmasked cytokine in a CD8 T cell pSTAT5 activity assay by at least 300-fold. In some embodiments, the masked cytokine has an EC50 of greater than the EC50 of an unmasked cytokine in a CD8 T cell pSTAT5 activity assay by at least 350-fold. In some embodiments, the masked cytokine has an EC50 of greater than the EC50 of an unmasked cytokine in a CD8 T cell pSTAT5 activity assay by at least 400-fold. In some embodiments, the masked cytokine has an EC50 of greater than the EC50 of an unmasked cytokine in a CD8 T cell pSTAT5 activity assay by at least 450-fold. In some embodiments, the masked cytokine has an EC50 of greater than the EC50 of an unmasked cytokine in a CD8 T cell pSTAT5 activity assay by at least 500-fold.

In some embodiments, wherein the masked cytokine has an in vitro cleavage efficiency Kcat/Km greater than or equal to 10⁴ M⁻¹s⁻¹ in the presence of an MMP. In some embodiments, wherein the masked cytokine has an in vitro cleavage efficiency Kcat/Km greater than or equal to 5×10⁴ M⁻¹s⁻¹ in the presence of an MMP. In some embodiments, wherein the masked cytokine has an in vitro cleavage efficiency Kcat/Km greater than or equal to 10¹ M⁻¹s⁻¹ in the presence of an MMP. In some embodiments, wherein the masked cytokine has an in vitro cleavage efficiency Kcat/Km greater than or equal to 5×10⁵ M⁻¹s⁻¹ in the presence of an MMP. In some embodiments, wherein the masked cytokine has an in vitro cleavage efficiency Kcat/Km greater than or equal to 10⁶ M⁻¹s⁻¹ in the presence of an MMP.

In some embodiments, at least 3% of the masked cytokine is cleaved upon contact with a tumor-specific protease. In some embodiments, at least 5% of the masked cytokine is cleaved upon contact with a tumor-specific protease. In some embodiments, at least 10% of the masked cytokine is cleaved upon contact with a tumor-specific protease. In some embodiments, at least 15% of the masked cytokine is cleaved upon contact with a tumor-specific protease. In some embodiments, at least 20% of the masked cytokine is cleaved upon contact with a tumor-specific protease. In some embodiments, at least 25% of the masked cytokine is cleaved upon contact with a tumor-specific protease. In some embodiments, at least 30% of the masked cytokine is cleaved upon contact with a tumor-specific protease. In some embodiments, at least 35% of the masked cytokine is cleaved upon contact with a tumor-specific protease. In some embodiments, at least 40% of the masked cytokine is cleaved upon contact with a tumor-specific protease. In some embodiments, at least 45% of the masked cytokine is cleaved upon contact with a tumor-specific protease. In some embodiments, at least 50% of the masked cytokine is cleaved upon contact with a tumor-specific protease. In some embodiments, at least 55% of the masked cytokine is cleaved upon contact with a tumor-specific protease. In some embodiments, at least 60% of the masked cytokine is cleaved upon contact with a tumor-specific protease. In some embodiments, at least 65% of the masked cytokine is cleaved upon contact with a tumor-specific protease. In some embodiments, at least 70% of the masked cytokine is cleaved upon contact with a tumor-specific protease. In some embodiments, at least 75% of the masked cytokine is cleaved upon contact with a tumor-specific protease.

In one aspect, the present invention provides, among other things, a nucleic acid encoding a masked cytokine described herein.

In one aspect, the present invention provides, among other things, a vector comprising a nucleic acid encoding a masked cytokine described herein.

In one aspect, the present invention provides, among other things, a host cell comprising a vector described herein.

In one aspect, the present invention provides, among other things, a method of producing a masked cytokine described herein culturing the host cell under a condition that produces the masked cytokine.

In one aspect, the present invention provides, among other things, a pharmaceutical composition comprising the masked cytokine described herein and a pharmaceutically acceptable carrier.

In one aspect, the present invention provides, among other things, a kit comprising the masked cytokine described herein.

In one aspect, the present invention provides, among other things, a method of treating or preventing a neoplastic disease in a subject, the method comprising administering to the subject an effective amount of the masked cytokine or the pharmaceutical composition described herein.

In one aspect, the present invention provides, among other things, a method of treating or preventing an inflammatory or autoimmune disease in a subject, the method comprising administering to the subject an effective amount of the masked cytokine or the pharmaceutical composition described herein.

In one aspect, the present invention provides a masked cytokine described herein, for use in medicine.

In one aspect, the present invention provides a masked cytokine described herein, for use in treating a neoplastic disease.

In one aspect, the present invention provides a masked cytokine described herein, for treating or preventing an inflammatory disease.

In one aspect, the present invention provides a masked cytokine described herein, for use in treating or preventing an autoimmune disease.

### BRIEF DESCRIPTION OF THE DRAWINGS

The drawings are for illustration purposes only, not for limitation.

FIGS. 1A-1B show schematics of exemplary masked IL-2 cytokine constructs. In FIG. 1A, the first polypeptide (chain A) comprises a targeting moiety, an Fc domain with “knob” and RF mutations, and an IL-2 polypeptide. The second polypeptide (chain B) comprises a targeting moiety, an Fc domain with “hole” mutations, a cleavable linker, and an anti-IL-2 VHH as the masking moiety. The masked cytokine in FIG. 1A has a third and fourth polypeptides (two copies of chain C), which comprises a variable light chain (VL) and constant light chain (CL). In FIG. 1B, the first polypeptide (chain A) comprises a targeting moiety, an Fc domain with “knob” and RF mutations, an IL-2 polypeptide and a cleavable linker. The second polypeptide (chain B) comprises a targeting moiety, an Fc domain with “hole” mutations, and an anti-IL-2 VHH as the masking moiety. The masked cytokine in FIG. 1A has a third and fourth polypeptides (two copies of chain C), which comprises a variable light chain (VL) and constant light chain (CL). Cross “X” indicates a location of cleavage site.

FIG. 2 shows exemplary in vitro measures of pSTAT5 activity in response to targeted masked cytokine activity in hPBMCs.

FIG. 3A is an exemplary graph showing % of cleaved molecules of targeted masked cytokines in tumor cells of different cancer indications. Cancer indications are plotted in the following order: melanoma, HNC (head and neck cancer), lung cancer, colon cancer, and RCC (renal cell cancer). FIG. 3B is an exemplary graph showing % of cleaved molecules of targeted masked cytokines in plasma from different cancer indications.

FIG. 4 shows exemplary measures of CD8 T cell profiles in splenic cells and in tumor microenvironment, respectively, in response to targeted masked cytokines.

FIG. 5 illustrates exemplary graphs showing % of active molecules in vivo resulting from protease cleavage of VHH masking moiety in tumor, plasma, and spleen.

FIG. 6 is an exemplary graph showing tumor volume of mice inoculated with tumor over 15-day treatment period with C5 and C6 constructs. The panel shows the % tumor growth inhibition for each treatment group.

FIG. 7A is an exemplary graph showing tumor volume of mice inoculated with tumor over 15-day treatment period. FIG. 7B is a series of exemplary graphs showing survival statistics of mice dosed with various treatment.

FIG. 8 is an exemplary graph showing the fold induction in TCR signaling for exemplary masked IL-2 cytokines in a PD-1/PD-L1 blockade bioassay.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

It is to be understood that this invention is not limited to particular compositions or biological systems, which can, of course, vary. It is also to be understood that the terminology used herein is for the purpose of describing particular embodiments only, and is not intended to be limiting.

As used in this specification and the appended claims, the singular forms “a,” “an,” and “the” include plural referents unless the content clearly dictates otherwise. Thus, for example, reference to “an IL-2 polypeptide” optionally includes a combination of two or more such polypeptides, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, the term “and/or” refers to any one of the items, any combination of the items, or all of the items with which the term is associated. For instance, the phrase “A, B, and/or C” is intended to encompass each of the following embodiments: A, B, and C; A, B, or C; A or B; A or C; B or C; A and B; A and C; B and C; A and B or C; B and A or C; C and A or B; A (alone); B (alone); and C (alone).

The term “antibody” includes polyclonal antibodies, monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)2, and Fv). The term “immunoglobulin” (Ig) is used interchangeably with “antibody” herein.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, such as the antigen binding region and/or the variable region of the intact antibody, and/or the constant region of the intact antibody. Examples of an antibody fragment include the Fc region of the antibody, a portion of the Fc region, or a portion of the antibody comprising the Fc region. Examples of antigen-binding antibody fragments include domain antibodies (dAbs), Fab, Fab′, F(ab′)2 and Fv fragments; diabodies; linear antibodies (see U.S. Pat. No. 5,641,870, Example 2; Zapata et ah, Protein Eng. 8(10): 1057-1062 [1995]); single-chain antibody molecules, and multispecific antibodies formed from antibody fragments, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), and a combination thereof. Single heavy chain antibodies or single light chain antibodies can be engineered, or in the case of the heavy chain, can be isolated from camelids, shark, libraries or mice engineered to produce single heavy chain molecules.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For example, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Antibody “effector functions” refer to those biological activities attributable to the Fc region (a native sequence Fc region or amino acid sequence variant Fc region) of an antibody and vary with the antibody isotype. Examples of antibody effector functions include: Clq binding and complement dependent cytotoxicity; Fc receptor binding; antibody-dependent cell-mediated cytotoxicity (ADCC); phagocytosis; down regulation of cell surface receptors (e.g., B cell receptors); and B cell activation.

“Attenuated IL-2” is used herein to refer to an IL-2 variant that comprises one or more mutations that reduce but do not eliminate binding to IL-2Rα. Present inventors discovered that “attenuated IL-2” can effectively expand tumor-specific T cells better than “not-alpha IL-2” and exhibit less regulatory T cell activity than “alpha-biased IL-2” counterpart. In some embodiments, attenuated IL-2 comprises mutations of F42E and C125A.

“Not-alpha IL-2” is used herein to refer to an IL-2 variant that comprises one or more mutations that eliminate binding to IL-2Rα. In some embodiments, not-alpha IL-2 comprises mutations of R38A, F42A, Y45A, E62A, and C125A relative wild-type IL-2.

“Alpha-biased IL-2” is used herein to refer to an IL-2 variant that comprises one or more mutation that reduce or eliminate binding to IL-2Rβγ. Alpha-biased IL-2 has “biased” affinity to the cells that constitutively express IL-2α (e.g., regulatory T-cells).

“Binding affinity” as used herein refers to the strength of the non-covalent interactions between a single binding site of a molecule (e.g., a cytokine) and its binding partner (e.g., a cytokine receptor). In some embodiments, the affinity of a binding protein (e.g., a cytokine) can generally be represented by a dissociation constant (Kd). Affinity can be measured by common methods known in the art, including those described herein.

An “isolated” nucleic acid molecule encoding the cytokine polypeptides described herein is a nucleic acid molecule that is identified and separated from at least one contaminant nucleic acid molecule with which it is ordinarily associated in the environment in which it was produced. In some embodiments, the isolated nucleic acid is free of association with all components associated with the production environment. The isolated nucleic acid molecules encoding the polypeptides and cytokine polypeptides herein is in a form other than in the form or setting in which it is found in nature. Isolated nucleic acid molecules therefore are distinguished from nucleic acid encoding the polypeptides and cytokine polypeptides herein existing naturally in cells.

The term “pharmaceutical formulation” refers to a preparation that is in such form as to permit the biological activity of the active ingredient to be effective, and that contains no additional components that are unacceptably toxic to a subject to which the formulation would be administered.

As used herein, the term “treatment” refers to clinical intervention designed to alter the natural course of the individual or cell being treated during the course of clinical pathology. Desirable effects of treatment include decreasing the rate of disease progression, ameliorating, or palliating the disease state, and remission or improved prognosis. An individual is successfully “treated”, for example, if one or more symptoms associated with a disorder (e.g., a neoplastic disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

As used herein, “in conjunction with” or “in combination with” refers to administration of one treatment modality in addition to another treatment modality. As such, “in conjunction with” or “in combination with” refers to administration of one treatment modality before, during or after administration of the other treatment modality to the individual.

As used herein, the term “prevention” includes providing prophylaxis with respect to occurrence or recurrence of a disease in an individual. An individual may be predisposed to, susceptible to a disorder, or at risk of developing a disorder, but has not yet been diagnosed with the disorder. In some embodiments, targeted cytokines described herein are used to delay development of a disorder.

As used herein, an individual “at risk” of developing a disorder may or may not have detectable disease or symptoms of disease and may or may not have displayed detectable disease or symptoms of disease prior to the treatment methods described herein. “At risk” denotes that an individual has one or more risk factors, which are measurable parameters that correlate with development of the disease, as known in the art. An individual having one or more of these risk factors has a higher probability of developing the disorder than an individual without one or more of these risk factors.

An “effective amount” refers to at least an amount effective, at dosages and for periods of time necessary, to achieve the desired or indicated effect, including a therapeutic or prophylactic result.

An effective amount can be provided in one or more administrations. A “therapeutically effective amount” is at least the minimum concentration required to affect a measurable improvement of a particular disorder. A therapeutically effective amount herein may vary according to factors such as the disease state, age, sex, and weight of the patient, and the ability of the antibody to elicit a desired response in the individual. A therapeutically effective amount may also be one in which any toxic or detrimental effects of the targeted cytokine are outweighed by the therapeutically beneficial effects. A “prophylactically effective amount” refers to an amount effective, at the dosages and for periods of time necessary, to achieve the desired prophylactic result. Typically, but not necessarily, since a prophylactic dose is used in subjects prior to or at the earlier stage of disease, the prophylactically effective amount can be less than the therapeutically effective amount.

“Chronic” administration refers to administration of the medicament(s) in a continuous as opposed to acute mode, so as to main the initial therapeutic effect (activity) for an extended period of time. “Intermittent” administration is treatment that is not consecutively done without interruption, but rather is cyclic in nature.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Disease: As used herein the term “disease” is a pathological condition, for example, one that can be identified by symptoms or other identifying factors as diverging from a healthy or a normal state. The term “disease” includes disorders, syndromes, conditions, and injuries. Diseases include, but are not limited to, proliferative, inflammatory, immune, metabolic, infectious, and ischemic diseases.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery).

Dosing interval: As used herein dosing interval in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Host cell: As used herein, the term “host cell” is an individual cell or a cell culture which can be or has been a recipient of any recombinant vector(s) or isolated polynucleotide(s), such as a vector or polynucleotide for an engineered cleavable Fc domain, or a masked cytokine of the present disclosure. A host cell includes cells transfected or infected in vivo or in vitro with a recombinant vector or a polynucleotide of the present invention. In some cases, the host cell is a mammalian host cell.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Moiety: As used herein, the term “moiety” refers to sub-structures which are part of a molecule.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Polypeptide: As used herein, the terms “Polypeptide,” “peptide,” and “protein” are used interchangeably herein to refer to a polymer of amino acid residues, e.g., a polymer of at least 3, 4, 5, 6, 7, 8, 9, 1o, or more amino acids. Throughout the present disclosure, standard three letter or single letter designations for amino acids are used. As used herein, the terms encompass amino acid chains of any length, including full-length proteins, wherein the amino acid residues are linked by covalent peptide bonds.

Polynucleotide: As used herein, the term “polynucleotide” refers to a polymeric form of nucleotides of at least 5, 6, 7, 8, 9 or 10 bases or base pairs in length, either ribonucleotides or deoxynucleotides or a modified form of either type of nucleotide and is meant to include single and double stranded forms of DNA and/or RNA. Throughout the present disclosure, the term is used interchangeably with “nucleic acid molecule”.

Subject: As used herein, the term “subject” refers to a human or any non-human animal (e.g., mouse, rat, rabbit, dog, cat, cattle, swine, sheep, horse, or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Vector: As used herein, the term “vector” refers to a macromolecule or complex of molecules comprising a polynucleotide to be delivered to a host cell or organism, either in vitro or in vivo, typically a virus or a plasmid.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

The masked cytokine constructs described herein are engineered to optimize binding between the masking moiety (e.g., anti-IL-2 VHH) and the cytokine (e.g., IL-2 polypeptide) to promote safe and effective targeted therapeutics for treating cancer. The present invention provides, among other things a masked cytokine comprising an attenuated IL-2 and a VHH masking moiety. A VHH masking moiety binds to the IL-2 cytokine and inhibits a biological activity of the cytokine in undesired targets. Upon cleavage in desired targets (e.g., tumor), the masking moiety is released from the cytokine, activating the function of the IL-2 polypeptide. In particular, the masked cytokine comprising an attenuated IL-2 and VHH masking moieties of the present invention is characterized with (1) effective masking efficiency such that IL-2 cytokine's function is inhibited in undesired targets; (2) efficient IL-2 activation by protease to release the VHH masking moiety; (3) selective IL-2 activation in tumor, and not in plasma; and (4) in vivo efficacy (e.g., high tumor growth inhibition).

### Interleukin 2 (IL-2)

Provided herein are IL-2 polypeptide or functional fragment thereof for use in any masked cytokine or cleavage product thereof. IL-2 plays an important role in cellular signalling, particularly in cells of the immune system by regulating activities of white blood cells. Suitable IL-2 polypeptides for use in the present invention can be any IL-2 polypeptide or functional fragment thereof. In some embodiments, the IL-2 polypeptide is naturally occurring IL-2. In some embodiments, the IL-2 polypeptide comprises one or more substitutions (e.g., an IL-2 mutein, or IL-2 variant), or truncated IL-2. In some embodiments, the IL-2 is a polypeptide that retains at least one property of IL-2 biological activity. In present application, the terms IL-2 polypeptide and IL-2 cytokine are used interchangeably.

In eukaryotic cells, naturally occurring IL-2 polypeptide is synthesized as a precursor polypeptide of 153 amino acids, which has SEQ ID NO: 1.

(SEQ ID NO: 1)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

This is then processed into mature IL-2 by the removal of amino acid residues 1-20. This results in a mature form of IL-2 consisting of 133 amino acids (amino acid residues 21-153) in SEQ ID NO: 2. In some embodiments, the IL-2 polypeptide is naturally occurring IL-2.

In some embodiments, the mature IL-2 polypeptide comprises a mutation at position 125 relative to SEQ ID NO: 2. In some embodiments, the mutation comprises C125A resulting in SEQ ID NO: 3.

### Attenuated IL-2

Present inventors discovered that the use of “attenuated IL-2 can effectively expand tumor-specific T cells better than other IL-2 variants (e.g., “not-alpha IL-2”), and exhibit less regulatory T cell activity than other IL-2 variants (e.g., “alpha-biased IL-2”). “Attenuated IL-2” refers to an IL-2 variant that comprises one or more mutations as compared to the wild-type IL-2 that reduce but do not eliminate binding to IL-2Rα. “Not-alpha IL-2” refers to an IL-2 variant that comprises one or more mutations that eliminate binding to IL-2Rα, and “alpha-biased IL-2” refers to an IL-2 variant that comprises one or more mutation that reduce or eliminate binding to IL-2Rβγ. In some embodiments, attenuated IL-2 comprises a mutation F42E. In some embodiments, attenuated IL-2 comprises mutations F42E and C125A.

In some embodiments, the amino acid substitutions reduce the affinity of the IL-2 polypeptide or functional fragment thereof for CD25 (IL-2Rα). In some embodiments, the IL-2 polypeptide or functional fragment thereof comprises mutations that reduce but do not eliminate affinity for IL-2Ra binding (e.g., attenuated IL-2).

In some embodiments, the IL-2 polypeptide or functional fragment thereof comprises an amino acid sequence having one or more amino acid substitutions as compared to the amino acid sequence of wild-type IL-2 of SEQ ID NO: 2 that reduces the affinity of the IL-2 polypeptide or functional fragment thereof for IL-2Rα (CD25).

In some embodiments, the IL-2 polypeptide or functional fragment thereof comprises amino acid sequence substitution C125A as compared to the amino acid sequence of SEQ ID NO: 2.

In some embodiments, the IL-2 polypeptide or functional fragment thereof comprises an amino acid sequence having about or at least about 85%, 86%, 87%, 88%, 89%, 90%, 91%, 92%, 93%, 94%, 95%, 96%, 97%, 98%, or 99% sequence identity to the amino acid sequence of SEQ ID NO: 2. In some embodiments, the IL-2 polypeptide or functional fragment thereof comprises an amino acid sequence having at least about 80% identity to SEQ ID NO: 2. In some embodiments, the IL-2 polypeptide or functional fragment thereof comprises an amino acid sequence having at least about 85% identity to SEQ ID NO: 2. In some embodiments, the IL-2 polypeptide or functional fragment thereof comprises an amino acid sequence having at least about 90% identity to SEQ ID NO: 2. In some embodiments, the IL-2 polypeptide or functional fragment thereof comprises an amino acid sequence having at least about 92% identity to SEQ ID NO: 2. In some embodiments, the IL-2 polypeptide or functional fragment thereof comprises an amino acid sequence having at least about 95% identity to SEQ ID NO: 2. In some embodiments, the IL-2 polypeptide or functional fragment thereof comprises an amino acid sequence having at least about 96% identity to SEQ ID NO: 2. In some embodiments, the IL-2 polypeptide or functional fragment thereof comprises an amino acid sequence having at least about 97% identity to SEQ ID NO: 2. In some embodiments, the IL-2 polypeptide or functional fragment thereof comprises an amino acid sequence having at least about 98% identity to SEQ ID NO: 2. In some embodiments, the IL-2 polypeptide or functional fragment thereof comprises an amino acid sequence having at least about 99% identity to SEQ ID NO: 2. In some embodiments, the IL-2 polypeptide or functional fragment thereof comprises an amino acid sequence of SEQ ID NO: 2.

In some embodiments, the IL-2 polypeptide comprises a substitution at position 42 relative to SEQ ID NO: 2. In some embodiments, the substitution is F42A. In some embodiments, the substitution is F42E. In some embodiments, the substitution is F42K. In some embodiments, the substitution is F42S. In some embodiments, the substitution is F42R. In some embodiments, the substitution is F42Q. In some embodiments, the substitution is F42Y.

In some embodiments, the IL-2 polypeptide comprises a sequence selected from Table 1. In some embodiments, the IL-2 polypeptide comprises SEQ ID NO: 2. In some embodiment, the IL-2 polypeptide comprises SEQ ID NO: 3. In some embodiments, the IL-2 polypeptide comprises SEQ ID NO: 4.

TABLE 1
Exemplary IL-2 Polypeptide Sequences.

| IL-2 | |
|---|---|
| Variant | Sequence |
| IL-2 WT | APTSSSTKKTQLQLEHLLLDLQMILNGINNYKNPKLTRMLTFKFYMPKKATELKHLQC |
| | LEEELKPLEEVLNLAQSKNFHLRPRDLISNINVIVLELKGSETTEMCEYADETATIVE |
| | FLNRWITFCQSIISTLT (SEQ ID NO: 2) |
| IL-2 V1 | APTSSSTKKTQLQLEHLLLDLQMILNGINNYKNPKLTRMLTFKFYMPKKATELKHLQC |
| (C125A) | LEEELKPLEEVLNLAQSKNFHLRPRDLISNINVIVLELKGSETTFMCEYADETATIVE |
| | FLNRWITFAQSIISTLT (SEQ ID NO: 3) |
| IL-2 V3 | APTSSSTKKTQLQLEHLLLDLQMILNGINNYKNPKLTRMLTEKFYMPKKATELKHLQC |
| (F42E, | LEEELKPLEEVLNLAQSKNFHLRPRDLISNINVIVLELKGSETTEMCEYADETATIVE |
| C125A) | FLNRWITFAQSIISTLT (SEQ ID NO: 4) |

“Functional fragments” of an IL-2 polypeptide comprise a portion of a full-length cytokine protein which retains or has modified cytokine receptor binding capability (e.g., within at least 50%, 80%, 90%, 95%, 96%, 97%, 98%, 99% or 100% activity compared to the full-length cytokine protein). Cytokine receptor binding capability can be shown, for example, by the capability of a cytokine to bind to the cytokine's cognate receptor or a component thereof (e.g., one or more chain(s) of a heterotrimeric receptor complex).

In some embodiments, the IL-2 polypeptide or functional fragment thereof is any naturally occurring interleukin-2 (IL-2) protein or modified variant thereof capable of binding to an interleukin-2 receptor, particularly the IL-2Rα chain. In the context of IL-2 polypeptide binding, the target protein could be IL-2R (comprising the IL-2Rα, IL-2Rβ, and IL-2Rγ chains), the IL-2Rα chain, the IL-2Rβ chain, or the IL-2Rα/β dimeric complex.

### VHH Masking Moieties

The present invention provides, among other things, novel anti-IL-2 antigen binding fragments (e.g., VHH) that can be used as masking moieties in any of the masked or targeted IL-2 cytokines described herein. A VHH masking moiety binds to the IL-2 cytokine and inhibits a biological activity of the cytokine. Upon cleavage, the masking moiety is released from the cytokine, activating the function of the IL-2 polypeptide. In particular, VHH masking moieties of the present invention are characterized with improved binding kinetics, effective IL-2 masking efficiency (i.e., inhibiting IL-2 in non-target environment), and activating IL-2 activity once released from IL-2 to promote an anti-tumor-response in tumor-microenvironment. The VHH masking moieties of the present invention effectively mask or inhibit IL2 activity such that the EC50 values based on pSTAT5 activity on CD8+ T cells of masked IL2 cytokines are greater than 300-fold as compared to an IL-2 cytokine without a masking moiety. In some embodiments, a masked cytokine has an EC50 value based on pSTAT5 activity on CD8+ T cells of greater than 300-fold as compared to an IL-2 cytokine without a masking moiety. But once the masking moiety is cleaved from the IL-2 cytokine molecule, IL-2 function is restored. Preferentially, % of cleaved molecules in tumor is greater than 5% in at least one of the cancer indications tested, and % of cleaved molecules is below the lower limit of quantitation (LLOQ) in plasma.

In some embodiments, anti-IL-2 VHHs of the present invention have improved stability, developability and manufacturability to be produced in large-scale for use in therapeutics. In some embodiments, anti-IL-2 VHHs of the present invention have reduced immunogenicity (i.e., induce minimal endotoxin or have reduced binding to pre-existing anti-drug antibodies).

In some embodiments, an anti-IL-2 VHH binds to wild-type IL-2 and not-alpha IL-2 with high affinity (e.g., <5 nM). In some embodiments, an anti-IL-2 VHH has a lower binding affinity to alpha-biased IL-2 (i.e., >500 nM) relative to a binding affinity to a wild-type IL-2 or not-alpha IL-2. In some embodiments, anti-IL-2 VHH binds to not-alpha IL-2 with KD of between 1 pM to 50 nM.

In some embodiments, the masking moiety is a heavy-chain-only antibody (VHH). In some embodiments, the masking moiety is an anti-IL-2 VHH antibody described herein.

VHH antibody (or nanobody) is the antigen binding fragment of heavy chain only antibodies. The VHH may be derived from, for example, an organism that produces VHH antibody such as a camelid, and a shark. In some cases, the VHH may be a recombinant VHH. VHH technology is based on fully functional antibodies from camelids that lack light chains. These heavy-chain antibodies contain a single variable domain (V_{H}H) and two constant domains (CH2 and CH3). A VHH comprises a single chain polypeptide having three CDRs and four framework regions (FRs1-4). As used herein, “framework region” or “FR” refers to a region in the variable domain which is located between the CDRs. As used herein, “complementary determining region” or “CDR” refers to variable regions in VHHs that contains the amino acid sequences capable of specifically binding to antigenic targets (e.g., cytokines). In present application, VHH and sdAb are used interchangeably.

In some embodiments, the VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains cytokine binding and specificity.

In some embodiments, the VHH masking moiety is linked to the Fc domain by a cleavable linker. In some embodiments, the VHH masking moiety is linked to the Fc domain by a cleavable linker described herein (e.g., a linker comprising a cleavage peptide described in Table 3). In some embodiments, the VHH masking moiety is linked to the Fc domain by a non-cleavable linker. In some embodiments the VHH masking moiety is linked the Fc domain by a non-cleavable linker described herein.

In some embodiments, the VHH antibody binds to an IL-2 polypeptide. In some embodiments, the VHH antibody binds to wild-type IL-2. In some embodiments, the VHH antibody binds to a variant of IL-2. In some embodiments, the VHH antibody binds to an engineered variant of IL-2.

In some embodiments, the VHH antibody comprises an amino acid sequence of SEQ ID NO: 5.

CDRs underlined.
(SEQ ID NO: 5)
EVQLVESGGGLVQPGGSLRLSCAASGSIFSINVMGWYRQAPGKQRELVAA
ISSGGSTNYADSVKGRFTISRDNAKNTVYLQMNSLKPEDTAVYYCMYASS
WYEDETDYWGQGTQVTVSS

In some embodiments, the VHH antibody comprises an amino acid sequence at least 85% identical to SEQ ID NO: 5. In some embodiments, the VHH antibody comprises an amino acid sequence at least 87% identical to SEQ ID NO: 5. In some embodiments, the VHH antibody comprises an amino acid sequence at least 90% identical to SEQ ID NO: 5. In some embodiments, the VHH antibody comprises an amino acid sequence at least 92% identical to SEQ ID NO: 5. In some embodiments, the VHH antibody comprises an amino acid sequence at least 93% identical to SEQ ID NO: 5. In some embodiments, the VHH antibody comprises an amino acid sequence at least 94% identical to SEQ ID NO: 5. In some embodiments, the VHH antibody comprises an amino acid sequence at least 95% identical to SEQ ID NO: 5. In some embodiments, the VHH antibody comprises an amino acid sequence at least 96% identical to SEQ ID NO: 5. In some embodiments, the VHH antibody comprises an amino acid sequence at least 97% identical to SEQ ID NO: 5. In some embodiments, the VHH antibody comprises an amino acid sequence at least 98% identical to SEQ ID NO: 5. In some embodiments, the VHH antibody comprises an amino acid sequence at least 99% identical to SEQ ID NO: 5.

In some embodiments, the VHH antibody comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In some embodiments, the VHH antibody comprises a CDR1 having an amino acid sequence that differs by 1 amino acid from SEQ ID NO: 6. In some embodiments, the VHH antibody comprises a CDR1 having an amino acid sequence that differs by 2 amino acids from SEQ ID NO: 6. In some embodiments, the VHH antibody comprises a CDR1 having an amino acid sequence that differs by 3 amino acids from SEQ ID NO: 6. In some embodiments, the VHH antibody comprises a CDR1 having an amino acid sequence that differs by 4 amino acids from SEQ ID NO: 6. In some embodiments, the VHH antibody comprises a CDR1 having an amino acid sequence that differs by 5 amino acids from SEQ ID NO: 6. In some embodiments, the VHH antibody comprises a CDR1 having an amino acid sequence that differs by 6 amino acids from SEQ ID NO: 6. In some embodiments, the VHH antibody comprises a CDR1 having an amino acid sequence that differs by 7 amino acids from SEQ ID NO: 6. In some embodiments, the VHH antibody comprises a CDR1 having an amino acid sequence that differs by 8 amino acids from SEQ ID NO: 6.

In some embodiments, the VHH antibody comprises a CDR2 having an amino acid sequence that differs by 1 amino acid from SEQ ID NO: 7. In some embodiments, the VHH antibody comprises a CDR2 having an amino acid sequence that differs by 2 amino acids from SEQ ID NO: 7. In some embodiments, the VHH antibody comprises a CDR2 having an amino acid sequence that differs by 3 amino acids from SEQ ID NO: 7. In some embodiments, the VHH antibody comprises a CDR2 having an amino acid sequence that differs by 4 amino acids from SEQ ID NO: 7. In some embodiments, the VHH antibody comprises a CDR2 having an amino acid sequence that differs by 5 amino acids from SEQ ID NO: 7. In some embodiments, the VHH antibody comprises a CDR2 having an amino acid sequence that differs by 6 amino acids from SEQ ID NO: 7. In some embodiments, the VHH antibody comprises a CDR2 having an amino acid sequence that differs by 7 amino acids from SEQ ID NO: 7. In some embodiments, the VHH antibody comprises a CDR2 having an amino acid sequence that differs by 8 amino acids from SEQ ID NO: 7. In some embodiments, the VHH antibody comprises a CDR2 having an amino acid sequence that differs by 9 amino acids from SEQ ID NO: 7. In some embodiments, the VHH antibody comprises a CDR2 having an amino acid sequence that differs by 10 amino acids from SEQ ID NO: 7.

In some embodiments, the VHH antibody comprises a CDR3 having an amino acid sequence that differs by 1 amino acid from SEQ ID NO: 8. In some embodiments, the VHH antibody comprises a CDR3 having an amino acid sequence that differs by 8 amino acids from SEQ ID NO: 8. In some embodiments, the VHH antibody comprises a CDR3 having an amino acid sequence that differs by 3 amino acids from SEQ ID NO: 8. In some embodiments, the VHH antibody comprises a CDR3 having an amino acid sequence that differs by 4 amino acids from SEQ ID NO: 8. In some embodiments, the VHH antibody comprises a CDR3 having an amino acid sequence that differs by 5 amino acids from SEQ ID NO: 8. In some embodiments, the VHH antibody comprises a CDR3 having an amino acid sequence that differs by 6 amino acids from SEQ ID NO: 8. In some embodiments, the VHH antibody comprises a CDR3 having an amino acid sequence that differs by 7 amino acids from SEQ ID NO: 8. In some embodiments, the VHH antibody comprises a CDR3 having an amino acid sequence that differs by 8 amino acids from SEQ ID NO: 8. In some embodiments, the VHH antibody comprises a CDR3 having an amino acid sequence that differs by 9 amino acids from SEQ ID NO: 8. In some embodiments, the VHH antibody comprises a CDR3 having an amino acid sequence that differs by 10 amino acids from SEQ ID NO: 8.

### Amino Acid Extensions to VHH

In some embodiments, the VHH masking moieties of the present invention are further modified. Some modifications provide modified molecules with a reduced ability to bind to pre-existing anti-drug antibodies (ADAs) as compared to the unmodified molecule. In other words, the modified molecule will bind with reduced affinity or avidity to a pre-existing ADA. Modifications can be selected from, for example and without limitation, C-terminal additions, extensions, deletions, or tags. Additional modifications are described in WO2013024059A2, the contents of which are incorporated herein by reference.

Additionally, the modified molecules may have an enhanced safety profile and fewer side effects than the unmodified molecule which does not comprise a C-terminal extension, addition, deletion, or tag. Administration of pharmaceutical compositions comprising the modified molecules can lead to improved immunogenicity and increased efficacy. A composition comprising the modified molecules can be advantageously used for repeated dosing to subjects who could develop autoantibodies to the unmodified molecules.

In some embodiments, the targeted masked cytokine comprises a modification. In some embodiments, the targeted masked cytokine comprises a C-terminal extension. In some embodiments, the targeted masked cytokine comprises a C-terminal addition. In some embodiments, the targeted masked cytokine comprises a C-terminal deletion. In some embodiments, the targeted masked cytokine comprises a C-terminal tag.

In some embodiments, the masking moiety comprises a modification. In some embodiments, the masking moiety comprises a C-terminal extension. In some embodiments, the masking moiety comprises a C-terminal addition. In some embodiments, the masking moiety comprises a C-terminal deletion. In some embodiments, the masking moiety comprises a C-terminal tag.

In some embodiments, the VHH antibody comprises a modification. In some embodiments, the VHH antibody comprises a C-terminal extension. In some embodiments, the VHH antibody comprises a C-terminal addition. In some embodiments, the VHH antibody comprises a C-terminal deletion. In some embodiments, the VHH antibody comprises a C-terminal tag.

In some embodiments, the C-terminal extension is selected from Table 2. In some embodiments, the C-terminal extension comprises an alanine. In some embodiments, the C-terminal extension comprises the sequence AS. In some embodiments, the C-terminal extension comprises the sequence AST. In some embodiments, the C-terminal extension comprises the sequence AAA. In some embodiments, the C-terminal extension comprises the sequence AH. In some embodiments, the C-terminal extension comprises the sequence GS. In some embodiments, the C-terminal extension comprises the sequence PP. In some embodiments, the C-terminal extension comprises the sequence PPP. In some embodiments, the C-terminal extension comprises the sequence GP. In some embodiments, the C-terminal modification comprises a deletion and an extension comprising the sequence GP.

In some embodiments, the C-terminal extension comprises alanine. In some embodiments, the C-terminal extension comprises serine. In some embodiments, the C-terminal extension comprises threonine. In some embodiments, the C-terminal extension comprises histidine. In some embodiments, the C-terminal extension comprises glycine. In some embodiments, the C-terminal extension comprises proline.

In some embodiments, the C-terminal extension comprises multiple alanine residues. In some embodiments, the C-terminal extension comprises multiple serine residues. In some embodiments, the C-terminal extension comprises multiple threonine residues. In some embodiments, the C-terminal extension comprises multiple histidine residues. In some embodiments, the C-terminal extension comprises multiple glycine residues. In some embodiments, the C-terminal extension comprises multiple proline residues.

TABLE 2
Exemplary VHH Antibody Sequences with Extensions.
Extensions are in bold.

| VHH | Extension | Sequence |
|---|---|---|
| VHH1 | No | EVQLVESGGGLVQPGGSLRLSCAASGSIFSINVMGWYRQAPGKQR |
| | Extension | ELVAAISSGGSTNYADSVKGRFTISRDNAKNTVYLQMNSLKPEDT |
| | | AVYYCMYASSWYEDETDYWGQGTQVTVSS (SEQ ID NO: 5) |
| VHH1-A | A | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSA (SEQ ID NO: 9) |
| VHH1-AS | AS | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSAS (SEQ ID NO: |
| | | 10) |
| VHH1-AST | AST | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSAST (SEQ ID NO: |
| | | 11) |
| VHH1-AAA | AAA | EVQLVESGGGLVQPGGSLRLSCAASGSIFSINVMGWYRQAPGKQR |
| | | ELVAAISSGGSTNYADSVKGRFTISRDNAKNTVYLOMNSLKPEDT |
| | | AVYYCMYASSWYEDETDYWGQGTQVTVSSAAA (SEQ ID NO: |
| | | 12) |
| VHH1-AH | AH | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSAH (SEQ ID NO: |
| | | 13) |
| VHH1-GS | GS | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSGS (SEQ ID NO: |
| | | 14) |
| VHH1-PP | PP | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSPP (SEQ ID NO: |
| | | 15) |
| VHH1-PPP | PPP | EVQLVESGGGLVQPGGSLRLSCAASGSIFSINVMGWYRQAPGKQR |
| | | ELVAAISSGGSTNYADSVKGRFTISRDNAKNTVYLQMNSLKPEDT |
| | | AVYYCMYASSWYEDETDYWGQGTQVTVSSPPP (SEQ ID NO: |
| | | 16) |
| VHH1-GP | S119G, P | EVQLVESGGGLVQPGGSLRLSCAASGSIFSINVMGWYRQAPGKQR |
| | | ELVAAISSGGSTNYADSVKGRETISRDNAKNTVYLQMNSLKPEDT |
| | | AVYYCMYASSWYEDETDYWGQGTQVTVSGP (SEQ ID NO: |
| | | 17) |

In some embodiments the VHH antibody with a C-terminal extension comprises SEQ ID NO: 9. In some embodiments the VHH antibody with a C-terminal extension comprises SEQ ID NO: 10. In some embodiments the VHH antibody with a C-terminal extension comprises SEQ ID NO: 11. In some embodiments the VHH antibody with a C-terminal extension comprises SEQ ID NO: 12. In some embodiments the VHH antibody with a C-terminal extension comprises SEQ ID NO: 13. In some embodiments the VHH antibody with a C-terminal extension comprises SEQ ID NO: 14. In some embodiments the VHH antibody with a C-terminal extension comprises SEQ ID NO: 15. In some embodiments the VHH antibody with a C-terminal extension comprises SEQ ID NO: 16. In some embodiments the VHH antibody with a C-terminal extension comprises SEQ ID NO: 17.

In some embodiments, the VHH antibody with a C-terminal extension comprises additional mutations. In some embodiments, the VHH antibody with a C-terminal extension comprises a sequence at least 85% identical to SEQ ID NO: 12. In some embodiments, the VHH antibody with a C-terminal extension comprises additional mutations. In some embodiments, the VHH antibody with a C-terminal extension comprises a sequence at least 87% identical to SEQ ID NO: 12. In some embodiments, the VHH antibody with a C-terminal extension comprises additional mutations. In some embodiments, the VHH antibody with a C-terminal extension comprises a sequence at least 90% identical to SEQ ID NO: 12. In some embodiments, the VHH antibody with a C-terminal extension comprises additional mutations. In some embodiments, the VHH antibody with a C-terminal extension comprises a sequence at least 92% identical to SEQ ID NO: 12. In some embodiments, the VHH antibody with a C-terminal extension comprises additional mutations. In some embodiments, the VHH antibody with a C-terminal extension comprises a sequence at least 93% identical to SEQ ID NO: 12. In some embodiments, the VHH antibody with a C-terminal extension comprises additional mutations. In some embodiments, the VHH antibody with a C-terminal extension comprises a sequence at least 94% identical to SEQ ID NO: 12. In some embodiments, the VHH antibody with a C-terminal extension comprises additional mutations. In some embodiments, the VHH antibody with a C-terminal extension comprises a sequence at least 95% identical to SEQ ID NO: 12. In some embodiments, the VHH antibody with a C-terminal extension comprises additional mutations. In some embodiments, the VHH antibody with a C-terminal extension comprises a sequence at least 96% identical to SEQ ID NO: 12. In some embodiments, the VHH antibody with a C-terminal extension comprises additional mutations. In some embodiments, the VHH antibody with a C-terminal extension comprises a sequence at least 97% identical to SEQ ID NO: 12. In some embodiments, the VHH antibody with a C-terminal extension comprises additional mutations. In some embodiments, the VHH antibody with a C-terminal extension comprises a sequence at least 98% identical to SEQ ID NO: 12. In some embodiments, the VHH antibody with a C-terminal extension comprises additional mutations. In some embodiments, the VHH antibody with a C-terminal extension comprises a sequence at least 99% identical to SEQ ID NO: 12.

### Other Masking Moiety Formats

In some embodiments, the anti-IL-2 VHH or antigen binding fragments thereof (e.g., CDR sequences) described herein are incorporated into an anti-IL-2 antigen binding molecule. In some embodiments, anti-IL-2 VHH or fragment thereof is incorporated into an anti-IL-2 antigen binding molecule selected from a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof.

In some embodiments, the anti-IL-2 antigen binding molecule incorporating the anti-IL-2 VHH sequences described herein can be an intact antibody, or a binding fragment thereof. In some embodiments, the binding fragments derived from an antibody include, but are not limited to, Fab, Fab′, F(ab′)2, scFv, and single-chain antibodies. In some embodiments, the anti-IL-2 antigen binding molecule incorporates the anti-IL-2 VHH sequences or antigen binding fragment thereof (e.g., CDRs) described herein.

In some embodiments, the anti-IL-2 antigen binding molecule derived from the anti-IL-2 VHH sequences is used as an antibody-based masking moiety in a masked cytokine.

In some embodiments, the anti-IL-2 antibody-based masking moiety comprises a Fab, a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, the anti-IL-2 antibody-based masking moiety is an scFv. In some embodiments, the anti-IL-2 antibody-based masking moiety is a Fab fragment. In some embodiments, the anti-IL-2 antibody-based masking moiety comprises one or more CDRs that bind to IL-2 (e.g., a CDR described herein). In some embodiments, the antibody-based masking moiety comprises a variable heavy chain (VH) that binds to IL-2. In some embodiments, the antibody-based masking moiety comprises a variable light chain (VL) that binds to IL-2. In some embodiments, the antibody-based masking moiety comprises a Fab-like bispecific antibodies (bsFab) that binds to IL-2. In some embodiments, the antibody-based masking moiety comprises a single-domain antibody-linked Fab (s-Fab) that binds to IL-2. In some embodiments, the antibody-based masking moiety comprises an antibody or a fragment thereof that binds to IL-2.

### Carrier Moiety

A long half-life in vivo is important for therapeutic proteins. Unfortunately, cytokines that are administered to a subject generally have a short half-life since they are normally cleared rapidly from the subject by mechanisms including clearance by the kidney and endocytic degradation. Thus, in the masked cytokine provided herein, a carrier moiety is linked to the cytokine or a masking moiety for the purpose of extending the half-life of the cytokine in vivo, among other things.

In some embodiments, the carrier moiety is a PEG molecule, an albumin, an albumin fragment, and antibody Fc domain, or an antibody or an antigen-binding fragment thereof. In some embodiments, the carrier moiety is an Fc domain. In some embodiments, the carrier moiety is a PEG molecule. In some embodiments, the carrier moiety is an albumin. In some embodiments, the carrier moiety is an albumin fragment. In some embodiments, the carrier moiety is an antibody or antigen-binding fragment thereof.

### Fc Domain

Provided herein are Fc domains for use in a masked cytokine or cleavage product thereof. In some embodiments, in the masked cytokine provided herein, an Fc domain is linked to the cytokine or a masking moiety for the purpose of extending the half-life of the cytokine in vivo, among other things.

As used herein, the term “Fc domain” refers to a polypeptide or a fragment from an immunoglobulin. The Fc domain may be any antibody or fragment thereof. The Fc domain is derived from an antibody or fragment thereof that naturally occur in an animal (e.g., a mammal) or is made by any technologies known in the art. In some embodiments, the Fc domain is a human Fc domain.

In some embodiments, the first Fc polypeptide and the second Fc polypeptide are linked via a linker such as a short peptide linker. In some embodiments, the linker is non-cleavable.

In some embodiments, an Fc domain comprises a first Fc polypeptide and a second Fc polypeptide.

An Fc domain or a fragment thereof that is capable of FcRn-mediated recycling, can be reduce or otherwise delay clearance of the targeted cytokine from a subject, thereby prolonging the half-life of the administered targeted cytokine. In some embodiments, the Fc domain or a fragment thereof is any antibody or fragment thereof that is capable of FcRn-mediated recycling, such as any heavy chain polypeptide or portion thereof (e.g., Fc domain or fragment thereof) that is capable of FcRn-mediated recycling.

The Fc domain or a fragment thereof can be any antibody or fragment thereof. However, in some embodiments, either a first Fc polypeptide or a second Fc polypeptide may does not bind to the FcRn receptor, such as a light chain polypeptide. For example, in some embodiments, a first Fc polypeptide does not directly interact with the FcRn receptor, but the targeted cytokine nonetheless has an extended half-life due to comprising a second Fc polypeptide that is capable of interacting with the FcRn receptor, such as by comprising a heavy chain polypeptide. It is recognized in the art that FcRn-mediated recycling requires binding of the FcRn receptor to the Fc region of the antibody or fragment thereof. For instance, studies have shown that residues 1253, S254, H435, and Y436 (numbering according to the Kabat EU index numbering system) are important for the interaction between the human Fc region and the human FcRn complex. See, e.g., Firan, M., et al., Int. Immunol. 13 (2001) 993-1002; Shields, R. L., et al, J. Biol. Chem. 276 (2001) 6591-6604). Various mutants of residues 248-259, 301-317, 376-382, and 424-437 (numbering according to the Kabat EU index numbering system) have also been examined and reported. Yeung, Y. A., et al. (J. Immunol. 182 (2009) 7667-7671.

In some embodiments, the antibody or fragment thereof comprises either a heavy chain polypeptide or a light chain polypeptide. In some embodiments, the antibody or fragment thereof comprises a portion of either a heavy chain polypeptide or a light chain polypeptide. In some embodiments, the antibody or fragment thereof comprises an Fc domain or fragment thereof. In some embodiments, the antibody or fragment thereof comprises a CH2 and CH3 domain or a fragment thereof. In some embodiments, the antibody or fragment thereof comprises the constant domain of the heavy chain polypeptide. In some embodiments, the antibody or fragment thereof comprises the constant domain of the light chain polypeptide. In some embodiments, the antibody or fragment thereof comprises a heavy chain polypeptide or fragment thereof (e.g., an Fc domain or fragment thereof). In some embodiments, the antibody or fragment thereof comprises a light chain polypeptide.

In some embodiments, the first and/or second Fc domains each contain one or more modifications that promote the non-covalent association of the first and the second Fc polypeptides. In some embodiments, the first Fc polypeptide comprises an IgG1 Fc domain or fragment thereof including the mutations Y349C; T366S; L368A; and Y407V to form a ‘hole’ in the first half-life extension domain and the second Fc polypeptide comprises an IgG1 Fc domain or fragment thereof including the mutations S354C and T366W to form the ‘knob’ in the second half-life extension domain.

In some embodiments, the first and second Fc polypeptides are each an IgG1, IgG2 or IgG4 Fc domain or fragment thereof. In some embodiments, the first and second Fc polypeptides are each an IgG1 Fc domain or fragment thereof. Human IgG1 Immunoglobulin heavy constant gamma 1 has the sequence:

(SEQ ID NO: 18)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

In some embodiments, the first and second Fc polypeptides are derived from the sequence for human IgG1 Immunoglobulin heavy constant gamma 1 having SEQ ID NO: 18 (the ‘parent sequence’), such that the first and second Fc polypeptides each comprise SEQ ID NO: 18 or fragment thereof, with one or more amino acid modifications.

In some embodiments, the first and Fc polypeptides each comprise the portion of SEQ ID NO: 18 shown above, optionally with one or more amino acid modifications, i.e.:

(SEQ ID NO: 19)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPG

In some embodiments, the first Fc polypeptide comprises an amino acid sequence having one or more modifications, such as one or more amino acid substitutions, additions, or deletions, as compared to the amino acid sequence of SEQ ID NO: 19. In some embodiments, the second Fc polypeptide comprises an amino acid sequence having one or more modifications, such as one or more amino acid substitutions, additions, or deletions, as compared to the amino acid sequence of any one of SEQ ID NO: 19. The one or more modifications can be any modifications or alterations described herein, including, in some embodiments, any modifications or alterations disclosed herein that promote heterodimerization of polypeptide chains and/or suppresses homodimerization of polypeptide chains, alter effector function, or enhance effector function.

In some embodiments, the Fc domain or fragment thereof comprises one or more amino acid substitutions altering effector function. In some embodiments, the half-life extension domain is an IgG1 Fc domain or fragment thereof and comprises one or more amino acid substitutions selected from the group consisting of N297A, N297G, N297Q, L234A, L235A, C220S, C226S, C229S, P238S, E233P, L234V, L234F, L235E, P331S, S267E, L328F, D265A, and P329G, numbered according to the Kabat EU numbering system. In some embodiments, the half-life extension domain is an IgG2 Fc domain or fragment thereof and comprises the amino substitution(s): V234A and G237A; H268Q, V309L, A330S, and A331S; and/or V234A, G237A, P238S, H268A, V309L, and A330S, numbered according to the Kabat EU numbering system. In some embodiments, the half-life extension domain is an IgG2 Fc domain or fragment thereof and comprises one or more amino acid substitutions selected from the group consisting of V234A, G237A, H268Q, V309L, A330S, A331S, P238S, H268A, and V309L, numbered according to the Kabat EU numbering system. In some embodiments, the half-life extension domain is an IgG4 Fc domain or fragment thereof and comprises the amino substitution(s): L235A, G237A, and E318A; S228P, L234A, and L235A; H268Q, V309L, A330S, and P331S; and/or S228P and L235A, numbered according to the Kabat EU numbering system. In some embodiments, the half-life extension domain is an IgG2 Fc domain or fragment thereof and comprises one or more amino acid substitutions selected from the group consisting of L235A, G237A, E318A, S228P, L234A, H268Q, V309L, A330S, and P331S, numbered according to the Kabat EU numbering system.

In some embodiments, the Fc domain or fragment thereof that comprises one or more amino acid substitutions enhancing effector function. In some embodiments, the half-life extension domain is an IgG1 Fc domain or fragment thereof and comprises the amino acid substitution(s): S298A, E333A, and K334A; S239D and I332E; S239D, A330L, and I332E; P2471 and A339D or A339Q; D280H and K290S; D280H, K290S, and either S298D or S298V; F243L, R292P, and Y300L; F243L, R292P, Y300L, and P396L; F243L, R292P, Y300L, V305I, and P396L; G236A, S239D, and I332E; K326A and E333A; K326W and E333S; K290E, S298G, and T299A; K290E, S298G, T299A, and K326E; K290N, S298G, and T299A; K290N, S298G, T299A, and K326E; K334V; L235S, S239D, and K334V; K334V and Q331M, S239D, F243V, E294L, or S298T; E233L, Q311M, and K334V; L234I, Q311M, and K334V; K334V and S298T, A330M, or A330F; K334V, Q311M, and either A330M or A330F; K334V, S298T, and either A330M or A330F; K334V, S239D, and either A330M or S298T; L234Y, Y296W, and K290Y, F243V, or E294L; Y296W and either L234Y or K290Y; S239D, A330S, and I332E, V264I; F243L and V264I; L328M; I332E; L328M and I332E; V264I and I332E; S239E and I332E; S239Q and I332E; S239E; A330Y; I332D; L328I and I332E; L328Q and I332E; V264T; V240I; V266I; S239D; S239D and I332D; S239D and I332N; S239D and I332Q; S239E and I332D; S239E and I332N; S239E and I332Q; S239N and I332D; S239N and I332E; S239Q and I332D; A330Y and I332E; V264I, A330Y, and I332E; A330L and I332E; V264I, A330L, and I332E; L234E, L234Y, or L234I; L235D, L235S, L235Y, or L235I; S239T; V240M; V264Y; A330I; N325T; I332E and L328D, L328V, L328T, or L328I; V264I, I332E, and either S239E or S239Q; S239E, V264I, A330Y, and I332E; A330Y, I332E, and either S239D or S239N; A330L, I332E, and either S239D or S239N; V264I, S298A, and I332E; S298A, I332E, and either S239D or S239N; S239D, V264I, and I332E; S239D, V264I, S298A, and I332E; S239D, V264I, A330L, and I332E; S239D, I332E, and A330I; P230A; P230A, E233D, and I332E; E272Y; K274T, K274E, K274R, K274L, or K274Y; F275W; N276L; Y278T; V302I; E318R; S324D, S324I or S324V; K326I or K326T; T335D, T335R, or T335Y; V240I and V266I; S239D, A330Y, I332E, and L234I; S239D, A330Y, I332E, and L235D; S239D, A330Y, I332E, and V240I; S239D, A330Y, I332E, and V264T; and/or S239D, A330Y, I332E, and either K326E or K326T, numbered according to the Kabat EU numbering system. In some embodiments, the Fc domain is an IgG1 Fc domain or fragment thereof and comprises one or more amino acid substitution(s) selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y.

In some embodiments, the Fc domain comprises one or more amino acid substitution(s) that enhance binding of the half-life extension domain to FcRn. In some embodiments, the one or more amino acid substitution(s) increase binding affinity of an Fc-containing polypeptide (e.g., a heavy chain polypeptide or an Fc domain or fragment thereof) to FcRn at acidic pH. In some embodiments, the half-life extension domain comprises one or more amino acid substitution(s) selected from the group consisting of M428F; T250Q and M428F; M252Y, S254T, and T256E; P257I and N434H; D376V and N434H; P257I and Q3111; N434A; N434W; M428F and N434S; V259I and V308F; M252Y, S254T, and T256E; V259I, V308F and M428F; T307Q and N434A; T307Q and N434S; T307Q, E380A, and N434A; V308P and N434A; N434H; and V308P.

### Knobs-into-Holes Approach

One strategy for promoting heterodimerization of two Fc polypeptides is an approach termed the “knobs-into-holes.”

In some embodiments, the targeted cytokine comprises a first Fc polypeptide and a second Fc polypeptide, each of which comprises a CH3 domain. In some embodiments, the Fc polypeptide comprising a CH3 domain is a heavy chain polypeptide or a fragment thereof (e.g., an Fc domain or fragment thereof). The CH3 domains of the two Fc polypeptides can be altered by the “knobs-into-holes” technology, which is described in detail with several examples in, e.g., WO 1996/027011; Ridgway, J. B. et al, Protein Eng. (1996) 9(7): 617-621; Merchant, A. M., et al, Nat. Biotechnol. (1998) 16(7): 677-681. See also Klein et al. (2012), MAbs, 4(6): 653-663. Using the knob-into-holes method, the interaction surfaces of the two CH3 domains are altered to increase the heterodimerization of the two half-life extension domains containing the two altered CH3 domains. This occurs by introducing a bulky residue into the CH3 domain of one of the half-life extension domains, which acts as the “knob.” Then, in order to accommodate the bulky residue, a “hole” is formed in the other half-life extension domain that can accommodate the knob. Either of the altered CH3 domains can be the “knob” while the other can be the “hole.” The introduction of a disulfide bridge further stabilizes the heterodimers (Merchant, A. M., et al, Nat. Biotechnol. (1998) 16(7); Atwell, S., et al, J. Mol. Biol. (1997) 270(1): 26-35) as well as increases yield.

It has been reported that heterodimerization yields above 97% can be achieved by introducing the S354C and T366W mutations in a heavy chain to create the “knob” and by introducing the Y349C, T366S, L368A, and Y407V mutations in a heavy chain to create the “hole” (numbering of the residues according to the Kabat EU numbering system). Carter et al. (2001), J. Immunol. Methods, 248: 7-15; Klein et al. (2012), MAbs, 4(6): 653-663.

In some embodiments comprising a first Fc polypeptide and a second Fc polypeptide, the first half-life Fc polypeptide comprises a heavy chain polypeptide or portion thereof (e.g., an Fc domain or fragment thereof) that comprises the amino acid mutations S354C and T366W (numbered according to the Kabat EU numbering system), and the second Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system). In some embodiments comprising a first Fc polypeptide and a second Fc polypeptide, the first Fc polypeptide comprises a heavy chain polypeptide or portion thereof (e.g., an Fc domain or fragment thereof) that comprises the amino acid mutations Y349C, T366S, L368A, and Y407V (numbered according to the Kabat EU numbering system), and the second Fc polypeptide comprises a heavy chain polypeptide or portion thereof (e.g., an Fc domain or fragment thereof) that comprises the amino acid mutations S354C and T366W (numbered according to the Kabat EU numbering system).

Additional examples of substitutions that can be made to form knobs and holes include those described in US20140302037A1, the contents of which are herein incorporated by reference. For example, in some embodiments, any of the following amino acid substitutions can be made to a first Fc polypeptide (“first domain”) and a paired second Fc polypeptide (“second domain”) that each contain an Fc domain: (a) Y407T in the first domain and T366Y in the second domain; (b) Y407A in the first domain and T366W in the second domain; (c) F405A in the first domain and T394W in the second domain; (d) F405W in the first domain and T394S in the second domain; (e) Y407T in the first domain and T366Y in the second domain; (f) T366Y and F405A in the first domain and T394W and Y407T in the second domain; (g) T366W and F405W in the first domain and T394S and Y407A in the second domain; (h) F405W and Y407A in the first domain and T366W and T394S in the second domain; or (i) T366W in the first domain and T366S, L368A, and Y407V in the second domain, numbered according to the Kabat EU numbering system.

In some embodiments, any of the following amino acid substitutions can be made to a first Fc polypeptide (“first domain”) and a paired second Fc polypeptide (“second domain”) that each contain an Fc domain: (a) Y407T in the second domain and T366Y in the first domain; (b) Y407A in the second domain and T366W in the first domain; (c) F405A in the second domain and T394W in the first domain; (d) F405W in the second domain and T394S in the first domain; (e) Y407T in the second domain and T366Y in the first domain; (f) T366Y and F405A in the second domain and T394W and Y407T in the first domain; (g) T366W and F405W in the second domain and T394S and Y407A in the first domain; (h) F405W and Y407A in the second domain and T366W and T394S in the first domain; or (i) T366W in the second domain and T366S, L368A, and Y407V in the first domain, numbered according to the Kabat EU numbering system.

In embodiments comprising a first Fc polypeptide and a second Fc polypeptide that each comprise an Fc domain, any of the heterodimerizing alterations described herein can be used in the Fc domains to promote heterodimerization of any of the targeted cytokines described herein.

In some embodiments, the first and second Fc polypeptides comprise SEQ ID NO: 19 with amino substitutions to promote association of the first and second Fc polypeptides according to the ‘knob into holes’ approach. In some embodiments, the sequence SEQ ID NO: 19 contains mutations Y349C; T366S; L368A; and Y407V (numbered according to the Kabat EU numbering system) to form the ‘hole’ in the first Fc polypeptide and mutations S354C and T366W (numbered according to the Kabat EU numbering system) to form the ‘knob’ in the second Fc polypeptide.

In some embodiments, the first Fc polypeptide comprises mutations Y349C; T366S; L368A; and Y407V relative to SEQ ID NO: 19. In some embodiments, the second Fc polypeptide comprises mutations S354C and T366W relative to SEQ ID NO: 19.

In some embodiments, the first and second half-life extension domains each further comprise amino substitution N297A, numbered according to the Kabat EU numbering system. In some embodiments, the first Fc polypeptide comprises mutations Y349C; T366S; L368A; Y407V, and N297A relative to SEQ ID NO: 19. In some embodiments, the second Fc polypeptide comprises mutations S354C, T366W, and N297A relative to SEQ ID NO: 19.

First Fc polypeptide (Y349C; T366S; L368A; Y407V and N297A) SEQ ID NO: 20:

(SEQ ID NO: 20)
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVCTLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG

In some embodiments, the first and second Fc polypeptides each further comprise the amino substitution I253A, numbered according to the Kabat EU numbering system.

In some embodiments, the first and second Fc polypeptides each further comprise both the amino substitutions N297A and I253A, numbered according to the Kabat EU numbering system. In some embodiments, the first Fc polypeptide comprises mutations Y349C; T366S; L368A; Y407V, N297A, and I253A relative to SEQ ID NO: 19. In some embodiments, the second Fc polypeptide comprises mutations S354C, T366W, N297A, and I253A relative to SEQ ID NO: 19

### RF Mutation or CH3 Domain Swap for Heterodimeric Protein Purification

Two immunoglobulin heavy chains that differ by at least one amino acid allows isolation of the antigen-binding protein based on a differential affinity of an immunoglobulin heavy chain and a modified or mutated immunoglobulin heavy chain toward an affinity reagent. The antigen-binding proteins that have IgG CH2 and CH3 regions with different affinities with respect to Protein A allow rapid isolation by differential binding of the IgG regions to Protein A.

In one embodiment, a second Fc polypeptide comprises a 95R modification (by IMGT exon numbering; 435R by EU numbering) in the CH3 region. In another embodiment, a second Fc polypeptide further comprises a 96F modification (IMGT; 436F by EU). In some embodiments, a first Fc polypeptide comprises wild-type CH2 and CH3 domains derived from IgG1 or IgG4, and a second Fc polypeptide comprises 95R/96F modifications by IMGT exon numbering. In some embodiments, a first Fc polypeptide comprises wild-type CH2 and CH3 domains derived from IgG1 or IgG4, and a second Fc polypeptide comprises 435R/436F modifications by EU numbering.

In some embodiments, a first Fc polypeptide comprises wild-type CH2 and CH3 domains derived from IgG1 or IgG4, and a second Fc polypeptide comprises CH3 domain derived from IgG3.

In some embodiments, a first Fc polypeptide or a second Fc polypeptide comprises amino acid sequence of SEQ ID NO: 21. SEQ ID NO: 21 comprises “knob mutations” with “RF mutations (435R/436F). In some embodiments, the first Fc polypeptide comprises S354C, T366W, N297A, H435R, and Y435F mutations relative to SEQ ID NO: 19.

(SEQ ID NO: 21)
DKTHTCPPCPAPELLGGPSVFLFPPKPKDTLMISRTPEVTCVVVDVSHED
PEVKFNWYVDGVEVHNAKTKPREEQYASTYRVVSVLTVLHQDWLNGKEYK
CKVSNKALPAPIEKTISKAKGQPREPQVYTLPPCRDELTKNQVSLWCLVK
GFYPSDIAVEWESNGQPENNYKTTPPVLDSDGSFFLYSKLTVDKSRWQQG
NVFSCSVMHEALHNRFTQKSLSLSPGGSPG

In some embodiments, the first Fc polypeptide comprises an amino acid sequence having about or at least about 85%, 86%, 87%, 88%, 89%, 90%, 91%, 92%, 93%, 94%, 95%, 96%, 97%, 98%, or 99% sequence identity to any one of the amino acid sequence of SEQ ID NO: 19 or SEQ ID NO: 20.

In some embodiments, the first Fc polypeptide or the second polypeptide comprises an amino acid sequence having about or at least about 85% sequence identity to amino acid sequence of SEQ ID NO: 19. In some embodiments, the first Fc polypeptide or the second polypeptide comprises an amino acid sequence having about or at least about 87% sequence identity to amino acid sequence of SEQ ID NO: 19. In some embodiments, the first Fc polypeptide or the second polypeptide comprises an amino acid sequence having about or at least about 90% sequence identity to amino acid sequence of SEQ ID NO: 19. In some embodiments, the first Fc polypeptide or the second polypeptide comprises an amino acid sequence having about or at least about 92% sequence identity to amino acid sequence of SEQ ID NO: 19. In some embodiments, the first Fc polypeptide or the second polypeptide comprises an amino acid sequence having about or at least about 93% sequence identity to amino acid sequence of SEQ ID NO: 19. In some embodiments, the first Fc polypeptide or the second polypeptide comprises an amino acid sequence having about or at least about 94% sequence identity to amino acid sequence of SEQ ID NO: 19. In some embodiments, the first Fc polypeptide or the second polypeptide comprises an amino acid sequence having about or at least about 95% sequence identity to amino acid sequence of SEQ ID NO: 19. In some embodiments, the first Fc polypeptide or the second polypeptide comprises an amino acid sequence having about or at least about 96% sequence identity to amino acid sequence of SEQ ID NO: 19. In some embodiments, the first Fc polypeptide or the second polypeptide comprises an amino acid sequence having about or at least about 97% sequence identity to amino acid sequence of SEQ ID NO: 19. In some embodiments, the first Fc polypeptide or the second polypeptide comprises an amino acid sequence having about or at least about 98% sequence identity to amino acid sequence of SEQ ID NO: 19. In some embodiments, the first Fc polypeptide or the second polypeptide comprises an amino acid sequence having about or at least about 99% sequence identity to amino acid sequence of SEQ ID NO: 19.

In some embodiments, the first Fc polypeptide or the second polypeptide comprises an amino acid sequence having about or at least about 85% sequence identity to amino acid sequence of SEQ ID NO: 20. In some embodiments, the first Fc polypeptide or the second polypeptide comprises an amino acid sequence having about or at least about 87% sequence identity to amino acid sequence of SEQ ID NO: 20. In some embodiments, the first Fc polypeptide or the second polypeptide comprises an amino acid sequence having about or at least about 90% sequence identity to amino acid sequence of SEQ ID NO: 20. In some embodiments, the first Fc polypeptide or the second polypeptide comprises an amino acid sequence having about or at least about 92% sequence identity to amino acid sequence of SEQ ID NO: 20. In some embodiments, the first Fc polypeptide or the second polypeptide comprises an amino acid sequence having about or at least about 93% sequence identity to amino acid sequence of SEQ ID NO: 20. In some embodiments, the first Fc polypeptide or the second polypeptide comprises an amino acid sequence having about or at least about 94% sequence identity to amino acid sequence of SEQ ID NO: 20. In some embodiments, the first Fc polypeptide or the second polypeptide comprises an amino acid sequence having about or at least about 95% sequence identity to amino acid sequence of SEQ ID NO: 20. In some embodiments, the first Fc polypeptide or the second polypeptide comprises an amino acid sequence having about or at least about 96% sequence identity to amino acid sequence of SEQ ID NO: 20. In some embodiments, the first Fc polypeptide or the second polypeptide comprises an amino acid sequence having about or at least about 97% sequence identity to amino acid sequence of SEQ ID NO: 20. In some embodiments, the first Fc polypeptide or the second polypeptide comprises an amino acid sequence having about or at least about 98% sequence identity to amino acid sequence of SEQ ID NO: 20. In some embodiments, the first Fc polypeptide or the second polypeptide comprises an amino acid sequence having about or at least about 99% sequence identity to amino acid sequence of SEQ ID NO: 20.

In some embodiments, the first Fc polypeptide or the second polypeptide comprises an amino acid sequence having about or at least about 85% sequence identity to amino acid sequence of SEQ ID NO: 21. In some embodiments, the first Fc polypeptide or the second polypeptide comprises an amino acid sequence having about or at least about 87% sequence identity to amino acid sequence of SEQ ID NO: 21. In some embodiments, the first Fc polypeptide or the second polypeptide comprises an amino acid sequence having about or at least about 90% sequence identity to amino acid sequence of SEQ ID NO: 21. In some embodiments, the first Fc polypeptide or the second polypeptide comprises an amino acid sequence having about or at least about 92% sequence identity to amino acid sequence of SEQ ID NO: 21. In some embodiments, the first Fc polypeptide or the second polypeptide comprises an amino acid sequence having about or at least about 93% sequence identity to amino acid sequence of SEQ ID NO: 21. In some embodiments, the first Fc polypeptide or the second polypeptide comprises an amino acid sequence having about or at least about 94% sequence identity to amino acid sequence of SEQ ID NO: 21. In some embodiments, the first Fc polypeptide or the second polypeptide comprises an amino acid sequence having about or at least about 95% sequence identity to amino acid sequence of SEQ ID NO: 21. In some embodiments, the first Fc polypeptide or the second polypeptide comprises an amino acid sequence having about or at least about 96% sequence identity to amino acid sequence of SEQ ID NO: 21. In some embodiments, the first Fc polypeptide or the second polypeptide comprises an amino acid sequence having about or at least about 97% sequence identity to amino acid sequence of SEQ ID NO: 21. In some embodiments, the first Fc polypeptide or the second polypeptide comprises an amino acid sequence having about or at least about 98% sequence identity to amino acid sequence of SEQ ID NO: 21. In some embodiments, the first Fc polypeptide or the second polypeptide comprises an amino acid sequence having about or at least about 99% sequence identity to amino acid sequence of SEQ ID NO: 21.

In some embodiments, the first polypeptide comprises amino acid sequence of SEQ ID NO: 20 and the second polypeptide comprises amino acid sequence of SEQ ID NO: 21. In some embodiments, the first polypeptide comprises amino acid sequence of SEQ ID NO: 21 and the second polypeptide comprises amino acid sequence of SEQ ID NO: 20.

### Linkers

Provided herein are linkers for use in a masked cytokine or cleavage product thereof. A linker as provided herein refers to a peptide of two more amino acids that is used to link two functional components together in the masked cytokines described herein.

In some embodiments, a masked cytokine comprises a first linker and a second linker, where at least the first linker or the second linker comprises a proteolytically cleavage peptide. In some embodiments, a masked cytokine comprises a first linker and a second linker, wherein both the first and the second linker are non-cleavable linkers.

In some embodiments, a first Fc polypeptide is linked to a cytokine or a variant thereof through a first linker. In some embodiments, a second Fc polypeptide is linked to a masking moiety through a second linker. In some embodiments, a first Fc polypeptide is linked to a cytokine or a variant thereof through a cleavable linker. In some embodiments, a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a non-cleavable linker. In some embodiments, a first Fc polypeptide is linked to a cytokine or a variant thereof through a non-cleavable linker and a second Fc polypeptide is linked to a masking moiety through a cleavable linker. In some embodiments, a first Fc polypeptide is linked to a cytokine or a variant thereof through a cleavable linker and a second Fc polypeptide is linked to a masking moiety through a cleavable linker. In some embodiments, a first Fc polypeptide is linked to a cytokine or a variant thereof through a non-cleavable linker and a second Fc polypeptide is linked to a masking moiety through a non-cleavable linker.

### Cleavable Linker

Cleavable linker in the present invention contains a cleavage peptide.

### Cleavage Peptides

A cleavage peptide is a polypeptide that includes a protease cleavage site, such that the cleavage peptide is proteolytically cleavable. Proteases are enzymes that cleave and hydrolyze the peptide bonds between two specific amino acid residues of target substrate proteins. A “cleavage site” as used herein refers to a recognizable site for cleavage of a portion of the cleavage peptide found in any of the linkers that comprise a cleavage peptide described herein. Thus, a cleavage site may be found in the sequence of a cleavage peptide as described herein. In the context of the present disclosure, the terms “cleavage peptide” and “cleavage peptide motif” are used interchangeably. In some embodiments, the cleavage site is an amino acid sequence that is recognized and cleaved by a cleaving agent.

In some embodiments, a cleavage site may be incorporated into a linker that is used to link two functional components together in the masked cytokines described herein.

In some embodiments, the protease cleavage site is a tumor-associated protease cleavage site. A “tumor-associated protease cleavage site” as provided herein is an amino acid sequence recognized by a protease whose expression is specific or upregulated for a tumor cell or tumor cell environment thereof.

The tumor cell environment is complex and can comprise multiple different proteases. As such, the precise site at which a given cleavage peptide will be cleaved in the tumor cell environment may vary between tumor types, between patients with the same tumor type and even between cleavage products formed in the same tumor dependent on the specific tumor cell environment. Moreover, even after cleavage, further modification of the initial cleavage product, e.g., by removal of one or two terminal amino acids, may occur by the further action of proteases in the tumor cell environment. A distribution of cleavage products can thus be expected to form in the tumor cell environment of a patient following administration of a single structure of a targeted cytokine as described herein.

It will be understood that a cleavage site as referred to herein refers to a site between two specific amino acid residues within the cleavage peptide that are a target for a protease known to be associated with a tumor cell environment. In this sense, there may be more than one cleavage site present in a cleavage peptide as described herein where different proteases cleave the cleavage peptide at different cleavage sites. It is also possible that more than one protease may act on the same cleavage site within a cleavage peptide. Discussion of protease cleavage sites can be found in the art.

Thus, the cleavage peptides disclosed herein may be cleaved by one or more proteases. In some embodiments, the protease cleavage site is a tumor-associated protease cleavage site. The tumor-associated protease cleavage site can be recognized by a tumor-associated protease. As non-limiting examples, the tumor-associated protease is a matrix metalloproteinase (MMP), selected from the group consisting of MMP1, MMP2, MMP3, MMP7, MMP8, MMP9, MMP10, MMP11, MMP12, MMP13, MMP14, MMP15, MMP16, MMP17, MMP19, MMP20, MMP21, MMP23A, MMP23B, MMP24, MMP25, MMP27, and MMP28. In one embodiment, the tumor-associated protease is MMP2. In another embodiment, the tumor-associated protease is MMP3. In some embodiments, the tumor-associated protease is MMP7. In yet another embodiment, the tumor-associated protease is MMP9. In yet another embodiment, the tumor-associated protease is MMP10. Other disease associated and tissue selective proteases include but are not limited to Cathepsins (Cathepsin B (catB), cathepsin D, cathepsin F, cathepsin K, cathepsin L, cathepsin V, cathepsin S, cathepsin W), ADAM, ADAMTS, Kallikreins 1 to 15, HTRA1-2-3, HGFAc, PRSS, TMPRSS, elastase, PR-3, granzymes (granzyme A, B, M, H and K), fibroblast activation proteins (FAP), plasmin, urokinase plasminogen activator (uPA), Tryptase, Caspase, Thrombin, Legumain, Chymase, Collagenase, napsin A, and matripatsel-2. In some embodiments, the disease associated and/or tissue selective protease is Cathepsins (Cathepsin B (catB)).

In some embodiments, the cleavage peptide is a substrate for a protease that is co-localized in a region or a tissue expressing the cytokine receptor.

In some embodiments, the cleavage peptide is a 5-mer (i.e. peptide 5 amino acids in length), 6-mer (i.e. peptide 6 amino acids in length), 7-mer (i.e. peptide 7 amino acids in length), 8-mer (i.e. peptide 8 amino acids in length), 9-mer (i.e. peptide 9 amino acids in length), 10-mer (i.e. peptide 10 amino acids in length), 11-mer (i.e. peptide 11 amino acids in length), 12-mer (i.e. peptide 12 amino acids in length), 13-mer (i.e. peptide 13 amino acids in length), 14-mer (i.e. peptide 14 amino acids in length), 15-mer (i.e. peptide 15 amino acids in length), 16-mer (i.e. peptide 16 amino acids in length), 17-mer (i.e. peptide 17 amino acids in length), or 18-mer (i.e. peptide 18 amino acids in length).

In some embodiments, the cleavage peptide is from 5 to 18 amino acids in length. In some embodiments, the cleavage peptide is from 6 to 10 amino acids in length.

In some embodiments, the cleavage peptide comprises SEQ ID NO: 22 (IHVTLKSL). In some embodiments, the cleavage peptide comprises SEQ ID NO: 23 (NSYTIKGL). In some embodiments, the cleavage peptide comprises SEQ ID NO: 24 (SNESLSLS). In some embodiments, the cleavage peptide comprises SEQ ID NO: 25 (QVSSSLSP). In some embodiments, the cleavage peptide comprises SEQ ID NO: 26 (PTSTSLSP). In some embodiments, the cleavage peptide comprises SEQ ID NO: 27 (ESLSLSEE). In some embodiments, the cleavage peptide comprises SEQ ID NO: 28 (ASLSLAPV). In some embodiments, the cleavage peptide comprises SEQ ID NO: 29 (SQESLSLS). In some embodiments, the cleavage peptide comprises SEQ ID NO: 30 (PLGL). In some embodiments, the cleavage peptide comprises SEQ ID NO: 31 (APAGLIVPYN). In some embodiments, the cleavage peptide comprises SEQ ID NO: 32 (PVSLRSGS). In some embodiments, the cleavage peptide comprises PANLVAPDP (SEQ ID NO: 33). In some embodiments, the cleavage peptide comprises MPYDLYHP (SEQ ID NO: 34). In some embodiments, the cleavage peptide comprises ISSGLLSGRS (SEQ ID NO: 35). In some embodiments, the cleavage peptide comprises RAAAVKSP (SEQ ID NO: 36). In some embodiments, the cleavage peptide comprises SEQ ID NO: 37 (RPLALWRS). In some embodiments, the cleavage peptide comprises SEQ ID NO: 38 (TQKPLGLS).). In some embodiments, the cleavage peptide comprises SEQ ID NO: 39 (RSKYLATA). In some embodiments, the cleavage peptide comprises GRPRHQGV (SEQ ID NO: 40). In some embodiments, the cleavage peptide comprises GLFG (SEQ ID NO: 41). In some embodiments, the cleavage peptide comprises GFLG (SEQ ID NO: 42). In some embodiments, the cleavage peptide comprises AGRRAAK (SEQ ID NO: 43). In some embodiments, the cleavage peptide comprises FRLWA (SEQ ID NO: 44). In some embodiments, the cleavage peptide comprises FRLWS (SEQ ID NO: 45). In some embodiments, the cleavage peptide comprises NFFGVGGE (SEQ ID NO: 46). In some embodiments, the cleavage peptide comprises PMKRLTLA (SEQ ID NO: 47). In some embodiments, the cleavage peptide comprises FPLATYAP (SEQ ID NO: 48). In some embodiments, the cleavage peptide comprises FLVGGASL (SEQ ID NO: 49). In some embodiments, the cleavage peptide comprises KPMQFLGD (SEQ ID NO: 50). In some embodiments, the cleavage peptide comprises GIVRAKGV (SEQ ID NO: 51). In some embodiments, the cleavage peptide comprises ALFKSSFP (SEQ ID NO: 52). In some embodiments, the cleavage peptide comprises SGRRSPGG (SEQ ID NO: 53). In some embodiments, the cleavage peptide comprises SLGRRPGG (SEQ ID NO: 54). In some embodiments, the cleavage peptide comprises SLSGRRGG (SEQ ID NO: 55). In some embodiments, the cleavage peptide comprises SLSLGRRG (SEQ ID NO: 56). In some embodiments, the cleavage peptide comprises SLSLSGRR (SEQ ID NO: 57). In some embodiments, the cleavage peptide comprises GGPRRL (SEQ ID NO: 58). In some embodiments, the cleavage peptide comprises GGPLRL (SEQ ID NO: 59). In some embodiments, the cleavage peptide comprises GGPKLL (SEQ ID NO: 60). In some embodiments, the cleavage peptide comprises GGPRNL (SEQ ID NO: 61). In some embodiments, the cleavage peptide comprises GGPRML (SEQ ID NO: 62). In some embodiments, the cleavage peptide comprises EHLRSPGG (SEQ ID NO: 63). In some embodiments, the cleavage peptide comprises FRSGVPGG (SEQ ID NO: 64). In some embodiments, the cleavage peptide comprises SLLLRTGN (SEQ ID NO: 65). In some embodiments, the cleavage peptide comprises AGLRSPGG (SEQ ID NO: 66). In some embodiments, the cleavage peptide comprises SLFRSAGP (SEQ ID NO: 67). In some embodiments, the cleavage peptide comprises SLFRAPGP (SEQ ID NO: 68). In some embodiments, the cleavage peptide comprises WLFRSPLG (SEQ ID NO: 69). In some embodiments, the cleavage peptide comprises SRLRSPQG (SEQ ID NO: 70). In some embodiments, the cleavage peptide comprises SLVLSGRR (SEQ ID NO: 71). In some embodiments, the cleavage peptide comprises KQLRHMRG (SEQ ID NO: 72). In some embodiments, the cleavage peptide comprises LSGRSDNH (SEQ ID NO: 73). In some embodiments, the cleavage peptide comprises LSGK (SEQ ID NO: 74). In some embodiments, the cleavage peptide comprises LSGR (SEQ ID NO: 75). In some embodiments, the cleavage peptide comprises RQARVVGG (SEQ ID NO: 76). In some embodiments, the cleavage peptide comprises RQRRVVGG (SEQ ID NO: 77). In some embodiments, the cleavage peptide comprises RQYRVVGG (SEQ ID NO: 78). In some embodiments, the cleavage peptide comprises SKGRSLIG (SEQ ID NO: 79). In some embodiments, the cleavage peptide comprises PRFKIIGG (SEQ ID NO: 80). In some embodiments, the cleavage peptide comprises KQLRVVNG (SEQ ID NO: 81). In some embodiments, the cleavage peptide comprises IQPRITGG (SEQ ID NO: 82). In some embodiments, the cleavage peptide comprises KQSRKFVP (SEQ ID NO: 83). In some embodiments, the cleavage peptide comprises GRQSRAGG (SEQ ID NO: 84). In some embodiments, the cleavage peptide comprises SGRSSPGG (SEQ ID NO: 85). In some embodiments, the cleavage peptide comprises SSGRSPGG (SEQ ID NO: 86). In some embodiments, the cleavage peptide comprises SLSGRSGG (SEQ ID NO: 87). In some embodiments, the cleavage peptide comprises SLSSGRSG (SEQ ID NO: 88). In some embodiments, the cleavage peptide comprises KLSLSGRS (SEQ ID NO: 89). In some embodiments, the cleavage peptide comprises PLRLSRA (SEQ ID NO: 90). In some embodiments, the cleavage peptide comprises PLKLSRA (SEQ ID NO: 91). In some embodiments, the cleavage peptide comprises PLGLSGRS (SEQ ID NO: 92). In some embodiments, the cleavage peptide comprises PLGLRSRA (SEQ ID NO: 93). In some embodiments, the cleavage peptide comprises PLGLKSRA (SEQ ID NO: 94). In some embodiments, the cleavage peptide comprises RGSRAG (SEQ ID NO: 95). In some embodiments, the cleavage peptide comprises RLSRGK (SEQ ID NO: 96). In some embodiments, the cleavage peptide comprises RGSRGG (SEQ ID NO: 97). In some embodiments, the cleavage peptide comprises KGSRAG (SEQ ID NO: 98). In some embodiments, the cleavage peptide comprises KLSRGK (SEQ ID NO: 99). In some embodiments, the cleavage peptide comprises GRSRAG (SEQ ID NO: 100). In some embodiments, the cleavage peptide comprises LRSRGK (SEQ ID NO: 101). In some embodiments, the cleavage peptide comprises GRSRGG (SEQ ID NO: 102). In some embodiments, the cleavage peptide comprises GKSRAG (SEQ ID NO: 103). In some embodiments, the cleavage peptide comprises LKSRGK (SEQ ID NO: 104). In some embodiments, the cleavage peptide comprises VPLSLYSGP (SEQ ID NO: 105). In some embodiments, the cleavage peptide comprises DSGGFMLT (SEQ ID NO: 106). In some embodiments, the cleavage peptide comprises HEQLTV (SEQ ID NO: 107). In some embodiments, the cleavage peptide comprises VPLSLY (SEQ ID NO: 108). In some embodiments, the cleavage peptide comprises DLLAVVAAS (SEQ ID NO: 109). In some embodiments, the cleavage peptide comprises GMPKDLYHAS (SEQ ID NO: 110). In some embodiments, the cleavage peptide comprises IVGRPRHQGV (SEQ ID NO: 111). In some embodiments, the cleavage peptide comprises MPYDLYHPS (SEQ ID NO: 112). In some embodiments, the cleavage peptide comprises VPLSLYSG (SEQ ID NO: 113). In some embodiments, the cleavage peptide comprises ISSGLLSGRSDQP (SEQ ID NO: 114). In some embodiments, the cleavage peptide comprises MPY (SEQ ID NO: 115). In some embodiments, the cleavage peptide comprises QQGNVFSC (SEQ ID NO: 116). In some embodiments, the cleavage peptide comprises LSLSGRS (SEQ ID NO: 117).

TABLE 3
Exemplary Cleavage Peptides

| | Sequence |
|---|---|
| | (* indicates |
| | a cleavage |
| | site within |
| SEQ ID | the cleavage |
| NO: | peptide) |
| 113 | VPLSLYSG |
| 116 | QQGNVFSC |
| 22 | IHVTLKSL |
| 23 | NSYTIKGL |
| 24 | SNESLSLS |
| 25 | QVSSSLSP |
| 26 | PTSTSLSP |
| 27 | ESLSLSEE |
| 28 | ASLSLAPV |
| 29 | SQESLSLS |
| 30 | PLGL |
| 31 | APAGLIVPYN |
| 32 | PVSLRSGS |
| 33 | PANLVAPDP |
| 34 | MPYD*LYHP |
| 35 | ISSGLL*SG*RS |
| 36 | RAAA*VKSP |
| 37 | RPLALWRS |
| 38 | TQKPLGLS |
| 39 | RSKYLATA |
| 40 | GRPRHQGV |
| 41 | GLFG |
| 42 | GFLG |
| 43 | AGRRAAK |
| 44 | FRLWA |
| 45 | FRLWS |
| 46 | NFFGVGGE |
| 47 | PMKRLTLA |
| 48 | FPLATYAP |
| 49 | FLVGGASL |
| 50 | KPMQFLGD |
| 51 | GIVRAKGV |
| 52 | ALFKSSFP |
| 53 | SGRRSPGG |
| 54 | SLGRRPGG |
| 55 | SLSGRRGG |
| 56 | SLSLGRRG |
| 57 | SLSLSGRR |
| 58 | GGPRRL |
| 59 | GGPLRL |
| 60 | GGPKLL |
| 61 | GGPRNL |
| 62 | GGPRML |
| 63 | EHLRSPGG |
| 64 | FRSGVPGG |
| 65 | SLLLRTGN |
| 66 | AGLRSPGG |
| 67 | SLFRSAGP |
| 68 | SLFRAPGP |
| 69 | WLFRSPLG |
| 70 | SRLRSPQG |
| 71 | SLVLSGRR |
| 72 | KQLRHMRG |
| 73 | LSGRSDNH |
| 74 | LSGK |
| 75 | LSGR |
| 76 | RQARVVGG |
| 77 | RQRRVVGG |
| 78 | RQYRVVGG |
| 79 | SKGRSLIG |
| 80 | PRFKIIGG |
| 81 | KQLRVVNG |
| 82 | IQPRITGG |
| 83 | KQSRKFVP |
| 84 | GROSRAGG |
| 85 | SGRSSPGG |
| 86 | SSGRSPGG |
| 87 | SLSGRSGG |
| 88 | SLSSGRSG |
| 117 | LSLSGRS |
| 89 | KLSLSGRS |
| 90 | PLRLSRA |
| 91 | PLKLSRA |
| 92 | PLGLSGRS |
| 93 | PLGLRSRA |
| 94 | PLGLKSRA |
| 95 | RGSRAG |
| 96 | RLSRGK |
| 97 | RGSRGG |
| 98 | KGSRAG |
| 99 | KLSRGK |
| 100 | GRSRAG |
| 101 | LRSRGK |
| 102 | GRSRGG |
| 103 | GKSRAG |
| 104 | LKSRGK |
| 105 | VPLSLYSGP |
| 106 | DSGG*FMLT |
| 107 | HEQ*LTV |
| 108 | VPLS*LY |
| 109 | DLLA*VVAAS |
| 110 | GMPKDLYHAS |
| 111 | IVGRPRHQGV |
| 112 | MPYDLYHPS |
| 114 | ISSGLLSGRSDQP |
| 115 | MPY |

Purely by way of example, in the above table, * indicates a known or observed protease cleavage site within the cleavage peptide.

In some embodiments, the cleavage peptide comprises an amino acid sequence selected from Table 3.

In some embodiments, the cleavable linker is between 2 and 25 amino acids in length. In some embodiments, the cleavable linker is between 3 and 21 amino acids in length. In some embodiments, the cleavable linker is between 3 and 18 amino acids in length. In some embodiments, the cleavable linker is between 5 and 18 amino acids in length. In some embodiments, the cleavable linker is between 3 and 8 amino acids in length. In some embodiments, the cleavable linker is between 4 and 6 amino acids in length.

In some embodiments, the cleavable linker is 15 amino acids in length. In some embodiments, the cleavable linker is 16 amino acids in length. In some embodiments, the cleavable linker is 17 amino acids in length. In some embodiments, the cleavable linker is 18 amino acids in length. In some embodiments, the cleavable linker is 19 amino acids in length. In some embodiments, the cleavable linker is 20 amino acids in length.

In some embodiments, a cleavable linker comprises a cleavage peptide listed in Table 3.

In some embodiments, the cleavable linker comprises a proteolytically cleavage peptide (CP) flanked on both sides by a spacer domain (SD) as shown in below formula:

SD-CP-SD

The cleavable linker comprises any cleavage peptide selected from Table 3.

In some embodiments, the cleavable linker comprises MPYDLYHPS (SEQ ID NO: 112). In some embodiments, the cleavable linker comprises MPY (SEQ ID NO: 115).

TABLE 4
Exemplary Cleavable Linkers

| SEQ ID | Sequence (cleavage |
|---|---|
| NO | peptide underlined) |
| 118 | GPPSGSSPMPYDLYHPSGGG |
| 119 | GPPSGSSPGMPYDLYHPSGGG |
| 120 | GGPMPYDLYHPSGGG |
| 121 | GSPMPYDLYHPSGGG |
| 122 | SSGGPMPYDLYHPSGGG |
| 123 | SGSSPGMPYDLYHPSGGG |
| 124 | GSSGPSGMPYDLYHPSGGG |
| 125 | GSSSGPGSMPYDLYHPSGGG |
| 126 | GPPSGSSPVPLSLYSGSGGG |
| 127 | GGSPVPLSLYSGSGGG |
| 128 | GSGGVPLSLYSGSGGG |
| 129 | GGSPGVPLSLYSGSGGG |
| 130 | GGSSGPVPLSLYSGSGGG |
| 131 | GGSSGSGVPLSLYSGSGGG |
| 132 | GGSPGSPVPLSLYSGSGGG |
| 133 | GSPGVPLSLYSGSSPMPYDLYHPSGG |
| 134 | GGSGGSVPLSLYSGP |
| 135 | GGSGGSGGSVPLSLYSGP |
| 136 | GGSGGSMPYDLYHPSGP |
| 137 | GGSGGSGGSMPYDLYHPSGP |
| 138 | GGSGGSDSGGFMLTSGP |
| 139 | GGSGGSGGSDSGGFMLTSGP |
| 140 | GGSGGSRAAAVKSPSGP |
| 141 | GGSGGSGGSRAAAVKSPSGP |
| 142 | GGSGGSISSGLLSGRSSGP |
| 143 | GGSGGSGGSISSGLLSGRSSGP |
| 144 | SPGGGGPMPYDLYHPSGGG |

In some embodiments, the cleavable linker comprises SPGGGGPMPYDLYHPSGGG (SEQ ID NO: 144).

### Spacer Domains

A spacer domain may consist of one or more amino acids. The function of the spacer domains, where present, is to link the proteolytically cleavage peptide (CP) to the other functional components in the constructs described herein.

It will be understood that spacer domains do not alter the biological interaction of the proteolytically cleavage peptide with proteases in the tumor-cell environment or in non-tumor cell environment. In other words, even in the presence of spacer domains the inventive proteolytically cleavage peptides disclosed herein retain their advantageous tumor specificity.

In some embodiments, the spacer domains flanking the proteolytically cleavage peptide are different.

In some embodiments, the spacer domains are rich in amino acid residues G, S and P.

In some embodiments, the spacer domains only include amino acid residue types selected from the group consisting of G, S and P.

In some embodiments, the cleavable linker comprises formula 12:

N′ SD1-CP-SD2 C′ (12)

where SD1 is a first spacer domain and SD2 is a second spacer domain.

In some embodiments, the cleavable linker comprises formula 12:

N′ SD1-CP-SD2 C′ (12)

In some embodiments, the first polypeptide chain comprises formula 7 and the second polypeptide chain comprises formula 13 below:

N′ HL1-non-cleavable L1-MM C′ (7)

N′ HL2-SD1-CP-SD2-C′ (13)

In some embodiments, the first polypeptide chain comprises formula 14 and the second polypeptide chain comprises formula 10 below:

N′ HL1-SD1-CP-SD2-MM C′ (14)

N′ HL2-non-cleavable L2-C C′ (10)

In some embodiments, SD1 consists of a glycine (G).

In some embodiments, the N-terminus of SD1 is a glycine (G).

In some embodiments, the first spacer domain (SD1) is between 3 and 10 amino acids in length. In some embodiments, the first spacer domain (SD1) is between 4 and 9 amino acids in length. In some embodiments, the first spacer domain (SD1) is between 3 and 6 amino acids in length.

In some embodiments, SD1 comprises a sequence selected from SEQ ID NOs: 145-161. In some embodiments, SD2 comprises a sequence selected from SEQ ID NOs: 145-161.

In some embodiments, the SD2 consists of GP.

In some embodiments, the C-terminus sequence of SD2 is -GP C′.

In some embodiments, the second spacer domain (SD2) is between 3 and 6 amino acids in length.

TABLE 5
Exemplary Spacer Domains

| SEQ ID NO of SD1 | Sequence |
|---|---|
| 145 | GGSSPP |
| 146 | GSGP |
| 147 | GSPG |
| 148 | GGSG |
| 149 | GPPSGSSPG |
| 150 | GPPSGSSP |
| 151 | GGPS |
| 152 | GSGPS |
| 153 | GSSGGP |
| 154 | GSP |
| 155 | GSGSPS |
| 156 | PSGSSPG |
| 157 | SGSPS |
| 158 | SPGGGGP |
| 159 | SGP |
| 160 | SGGG |
| 161 | GSGGG |

Exemplary combinations of SD1 and SD2 in a cleavable linker are shown in Table 6.

TABLE 6
Cleavable Linker Structure

| Linker | | |
|---|---|---|
| structure | SD1 sequence | SD2 sequence |
| SD1-CP-SD2 | GPPSGSSPG | SGGG |
| | SEQ ID NO: 149 | SEQ ID NO: 160 |
| SD1-CP-SD2 | GPPSGSSPG | GSGGG |
| | SEQ ID NO: 149 | SEQ ID NO: 161 |
| SD1-CP-SD2 | GPPSGSSP | SGGG |
| | SEQ ID NO: 150 | SEQ ID NO: 160 |
| SD1-CP-SD2 | GGSSPP | SGP |
| | SEQ ID NO: 145 | SEQ ID NO: 159 |
| SD1-CP-SD2 | GSPG | SGP |
| | SEQ ID NO: 147 | SEQ ID NO: 159 |
| SD1-CP-SD2 | GGSG | SGP |
| | SEQ ID NO: 148 | SEQ ID NO: 159 |
| SD1-CP-SD2 | GSGP | SGP |
| | SEQ ID NO: 146 | SEQ ID NO: 159 |
| SD1-CP-SD2 | GGPS | SGP |
| | SEQ ID NO: 151 | SEQ ID NO: 159 |
| SD1-CP-SD2 | GSGPS | SGP |
| | SEQ ID NO: 152 | SEQ ID NO: 159 |
| SD1-CP-SD2 | GSSGGP | SGP |
| | SEQ ID NO: 153 | SEQ ID NO: 159 |
| SD1-CP-SD2 | GSP | SGP |
| | SEQ ID NO: 154 | SEQ ID NO: 159 |
| SD1-CP-SD2 | GSGSPS | SGP |
| | SEQ ID NO: 155 | SEQ ID NO: 159 |
| SD1-CP-SD2 | G | SGP |
| | | SEQ ID NO: 159 |

### Non-Cleavable Linker

In some embodiments, the non-cleavable linker is between 2 and 25 amino acids in length. In some embodiments, the non-cleavable linker is between 3 and 21 amino acids in length. In some embodiments, the non-cleavable linker is between 3 and 18 amino acids in length. In some embodiments, the non-cleavable linker is between 5 and 18 amino acids in length. In some embodiments, the non-cleavable linker is between 3 and 8 amino acids in length. In some embodiments, the non-cleavable linker is between 4 and 6 amino acids in length.

In some embodiments, the non-cleavable linker is 15 amino acids in length. In some embodiments, the non-cleavable linker is 16 amino acids in length. In some embodiments, the non-cleavable linker is 17 amino acids in length. In some embodiments, the non-cleavable linker is 18 amino acids in length. In some embodiments, the non-cleavable linker is 19 amino acids in length. In some embodiments, the non-cleavable linker is 20 amino acids in length.

In some embodiments, the non-cleavable linker is rich in amino acid residues G, S and P. In some embodiments, the non-cleavable linker only includes amino acid residue types selected from the group consisting of G, S and P. In some embodiments, the non-cleavable linker includes a ‘GS’ repeat. In some embodiments, the non-cleavable linker includes an N′ terminal ‘P’ residue.

In some embodiments, the non-cleavable linker comprises an amino acid sequence as shown in SEQ ID NO: 162 (PGSGS). In some embodiments, the non-cleavable linker comprises an amino acid sequence as shown in SEQ ID NO: 163 (GGSSPPGGGSSGGGSGP). In some embodiments, the non-cleavable linker comprises an amino acid sequence as shown in SEQ ID NO: 164 (GGGGS). In some embodiments, the non-cleavable linker comprises an amino acid sequence as shown in SEQ ID NO: 165 (GGGGSGGGGS). In some embodiments, the non-cleavable linker comprises an amino acid sequence as shown in SEQ ID NO: 166 (GGSGGGSGGGGGS). In some embodiments, the non-cleavable linker comprises an amino acid sequence as shown in SEQ ID NO: 167 (GGSGGSGGSGGSGGSSGP). In some embodiments, the non-cleavable linker comprises an amino acid sequence as shown in SEQ ID NO: 168 (PGGSGP).

In some embodiments, the non-cleavable linker comprises an amino acid sequence GGS.

In some embodiments, the non-cleavable linker includes [(G)ₙS], where n=4 or 5.

In some embodiments, it is desirable for the first and second polypeptide chains to be of the same or a similar length to facilitate the first half life extension domain associating with the second half life extension domain and the masking moiety masking the cytokine or functional fragment thereof in the assembled construct. As such where the masking moiety is a shorter amino acid sequence than the cytokine or functional fragment thereof, the difference in length may be compensated fully or in part by using a longer linker L1.

### Targeting Moieties

In some embodiments, the masked IL-2 cytokines described herein further comprise a targeting moiety (e.g., a targeted cytokine). Accordingly, the present invention also provides, a targeted cytokine which comprises a targeting moiety, a cytokine or a variant thereof (e.g., an IL-2 polypeptide), a masking moiety (e.g., an anti-IL-2 VHH described herein), and a carrier moiety. In some embodiments, a targeted cytokine comprises a targeting moiety, an IL-2 polypeptide, a masking moiety, and an Fc domain. Targeted cytokines of the present invention become active at the site of disease and are able to specifically target a cell of interest for effective treatment of cancer without causing undesired side effects.

Targeted IL-2 polypeptides according to the disclosure can combine an IL-2 polypeptide or functional fragment thereof as described anywhere herein; a masking moiety as described anywhere herein; first and second Fc domains as described anywhere herein; cleavable and non-cleavable linkers as described anywhere herein; and targeting moieties as described anywhere herein.

Provided herein is a targeted cytokine that comprises a targeting moiety. In some embodiments, a targeting moiety comprises an antigen-binding moiety that binds to an antigen expressed on the surface of a target cell.

In some embodiments, the targeting moiety comprises an antigen-binding moiety, wherein the antigen is expressed on an immune cell. In some embodiments, the targeting moiety comprises an antigen-binding moiety, wherein the antigen is selected from PD-1, PD-L1, CTLA-4, TIGIT, TIM-3, LAG-3, OX40, DR5, ICOS, GITR, CD73, CD39, CD25, CD16a, CD8, KLRC1, KLRD1, KLRB1, CD40, CD137, CD28 and CD16b.

In some embodiments, a targeting moiety specifically binds PD-1, PD-L1, PD-L2, CTLA-4, TIGIT, TIM-3, LAG-3, CD25, CD16a, CD16b, OX40, DR5, ICOS, GITR, NKG2D, KLRC1, KLRD1, KLRB1, NKP44, NKP30, BCMA, human epidermal growth factor receptor 2 (HER2), MICA, DLK1, human epidermal growth factor receptor 3 (HER3), delta-like protein 3 (DLL3), delta-like protein 4 (DLL4), epidermal growth factor receptor (EGFR), glypican-3 (GPC3), c-MET, vascular endothelial growth factor receptor 1 (VEGF R1), vascular endothelial growth factor receptor 2 (VEG FR2), Nectin-4, Liv-1, glycoprotein NMB (GPNMB), prostate specific membrane antigen (PSMA), Trop-2, carbonic anhydrase IX (CA9), endothelin B receptor (ETBR), six transmembrane epithelial antigen of the prostate 1 (STEAP1), NAPI2B, folate receptor alpha (FR-a), SLIT and NTRK-like protein 6 (SLITRK6), carbonic anhydrase VI (CA6), ectonucleotide pyrophosphatase/phosphodiesterase family member 3 (ENPP3), mesothelin, trophoblast glycoprotein (TPBG), CD19, CD8, CD20, CD22, CD28, CD33, CD39, CD40, CD56, CD66e, CD70, CD73, CD74, CD79b, CD98, CD123, CD137, CD138, CD352, CD47, signal-regulatory protein alpha (SIRPα), Claudin 18.2, Claudin 6, 5T4, fibroblast activation protein alpha (FAPα), fibronectin, the melanoma-associated chondroitin sulfate proteoglycan (MCSP), epithelial cellular adhesion molecule (EPCAM), or combinations thereof.

In some embodiments, a targeting moiety binds a tumor-associated antigen. In some embodiments, a targeting moiety is an antibody or an antigen binding fragment that binds a tumor-associated antigen. In some embodiments, a targeting moiety is a bispecific antibody or an antigen binding fragment that binds a tumor-associated antigen. In some embodiments, a targeting moiety is an anti-alpha-fetoprotein (AFP) antibody or a fragment thereof. In some embodiments, a targeting moiety is an anti-B2M antibody or a fragment thereof. In some embodiments, a targeting moiety is an anti-beta-human chorionic gonadotropin (beta-hCG) antibody or a fragment thereof. In some embodiments, a targeting moiety is an anti-CD117 antibody or a fragment thereof. In some embodiments, a targeting moiety is an anti-CD19 antibody or a fragment thereof. In some embodiments, a targeting moiety is an anti-CD20 antibody or a fragment thereof. In some embodiments, a targeting moiety is an anti-CD22 antibody or a fragment thereof. In some embodiments, a targeting moiety is an anti-CD25 antibody or a fragment thereof. In some embodiments, a targeting moiety is an anti-CD30 antibody or a fragment thereof. In some embodiments, a targeting moiety is an anti-CD33 antibody or a fragment thereof. In some embodiments, a targeting moiety is an anti-CD151 antibody or a fragment thereof. In some embodiments, a targeting moiety is an anti-MUC-1 antibody or a fragment thereof.

### PD-1 Targeting Moiety

In some embodiments, a targeting moiety specifically binds PD-1. In some embodiments, a targeting moiety is an anti-PD1 Fab. In some embodiments, a targeting moiety is an anti-PD1 scFv.

In some embodiments, the targeting moiety is derived from an anti-PD1 antibody. In some embodiments, the targeting moiety is derived from pembrolizumab or nivolumab. In some embodiments, the targeting moiety is derived from pembrolizumab. In some embodiments, the targeting moiety is derived from nivolumab.

In some embodiments, a targeting moiety binds PD-L1.

In some embodiments, a targeting moiety comprises an agent, a peptide, or a polypeptide that specifically binds to a target.

In some embodiments, a targeting moiety comprises a Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, or a combination thereof. In some embodiments, a targeting moiety comprises a Fab. In some embodiments, a targeting moiety comprises a single chain Fv (scFv). In some embodiments, a targeting moiety comprises a single domain antibody (VHH). In some embodiments, a targeting moiety comprises one or more CDRs. In some embodiments, a targeting moiety comprises a variable heavy chain (VH). In some embodiments, a targeting moiety comprises a variable light chain (VL). In some embodiments, a targeting moiety comprises a Fab-like bispecific antibodies (bsFab). In some embodiments, a targeting moiety comprises a single-domain antibody-linked Fab (s-Fab). In some embodiments, a targeting moiety comprises an antibody or a fragment thereof.

In some embodiments, the targeting moiety comprises a heavy chain variable region or a light chain variable region of pembrolizumab.

In some embodiments, a targeting moiety comprises a heavy chain variable region of

(SEQ ID NO: 169)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 169. In some embodiments, a targeting moiety comprises an amino acid sequence of SEQ ID NO: 169.

In some embodiments, a targeting moiety comprises a heavy chain variable and constant region of:

(SEQ ID NO: 170)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSC.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 170. In some embodiments, a targeting moiety comprises an amino acid sequence of SEQ ID NO: 170.

In some embodiments, a targeting moiety comprises a heavy chain of

(SEQ ID NO: 171)
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 171. In some embodiments, a targeting moiety comprises an amino acid sequence having of SEQ ID NO: 171.

In some embodiments, a targeting moiety comprises a heavy chain CDR1 sequence of GYTFTNYY (SEQ ID NO: 172), a heavy chain CDR2 sequence of INPSNGGT (SEQ ID NO: 173), and a heavy chain CDR3 sequence of ARRDYRFDMGFDY (SEQ ID NO: 174).

In some embodiments, a targeting moiety comprises a light chain variable region of

(SEQ ID NO: 175)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKTSENLYFQ.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 175. In some embodiments, a targeting moiety comprises an amino acid sequence identical to SEQ ID NO: 175.

In some embodiments, a targeting moiety comprises a light chain of

(SEQ ID NO: 176)
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 176. In some embodiments, a targeting moiety comprises an amino acid sequence identical to SEQ ID NO: 176.

In some embodiments, a targeting moiety comprises a light chain CDR1 sequence of KGVSTSGYSY (SEQ ID NO: 177), a light chain CDR2 sequence of LAS (SEQ ID NO: 178), and a light chain CDR3 sequence of QHSRDLPLT (SEQ ID NO: 179).

In some embodiments, a targeting moiety comprises a HCDR1 of SEQ ID NO: 172, a HCDR2 of SEQ ID NO: 173, a HCDR3 of SEQ ID NO: 174, a LCDR1 of SEQ ID NO: 177, a LCDR2 of SEQ ID NO: 178, and a LCDR3 of SEQ ID NO: 179.

In some embodiments, the targeting moiety comprises a heavy chain variable region or a light chain variable region of nivolumab.

In some embodiments, a targeting moiety comprises a heavy chain variable region of

(SEQ ID NO: 180)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 180. In some embodiments, a targeting moiety comprises an amino acid sequence identical to SEQ ID NO: 180.

In some embodiments, a targeting moiety comprises a heavy chain of

(SEQ ID NO: 181)

QVQLVESGGG VVQPGRSLRL DCKASGITFS NSGMHWVRQA
PGKGLEWVAV IWYDGSKRYY ADSVKGRFTI SRDNSKNTLF
LQMNSLRAED TAVYYCATND DYWGQGTLVT VSSASTKGPS
VFPLAPCSRS TSESTAALGC LVKDYFPEPV TVSWNSGALT
SGVHTFPAVL QSSGLYSLSS VVTVPSSSLG TKTYTCNVDH
KPSNTKVDKR VESKYGPPCP PCPAPEFLGG PSVFLFPPKP
KDTLMISRTP EVTCVVVDVS QEDPEVQFNW YVDGVEVHNA
KTKPREEQFN STYRVVSVLT VLHQDWLNGK EYKCKVSNKG
LPSSIEKTIS KAKGQPREPQ VYTLPPSQEE MTKNQVSLTC
LVKGFYPSDI AVEWESNGQP ENNYKTTPPV LDSDGSFFLY
SRLTVDKSRW QEGNVFSCSV MHEALHNHYT QKSLSLSLGK.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 181. In some embodiments, a targeting moiety comprises an amino acid sequence identical to SEQ ID NO: 181.

In some embodiments, a targeting moiety comprises a heavy chain CDR1 sequence of GITFSNSG (SEQ ID NO: 182), a heavy chain CDR2 sequence of VIWYDGSKRYYADSVKG (SEQ ID NO: 183), and a heavy chain CDR3 sequence of ATNDDY (SEQ ID NO: 184).

In some embodiments, the targeting moiety comprises a heavy chain variable region and a CH1 domain comprising SEQ ID NO: 185.

(SEQ ID NO: 185)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKKVEPKSC.

In some embodiments, a targeting moiety comprises a light chain variable region of

(SEQ ID NO: 186)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIK.

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 186. In some embodiments, a targeting moiety comprises an amino acid sequence identical to SEQ ID NO: 186.

In some embodiments, a targeting moiety comprises a light chain of EIVLTQSPAT LSLSPGERAT LSCRASQSVS SYLAWYQQKP GQAPRLLIYD ASNRATGIPA RFSGSGSGTD FTLTISSLEP EDFAVYYCQQ SSNWPRTFGQ GTKVEIKRTV AAPSVFIFPP SDEQLKSGTA SVVCLLNNFY PREAKVQWKV DNALQSGNSQ ESVTEQDSKD STYSLSSTLT LSKADYEKHK VYACEVTHQG LSSPVTKSFN RGE (SEQ ID NO: 187).

In some embodiments, a targeting moiety comprises an amino acid sequence having at least about 80%, 85%, 90%, 91%, 92% 93%, 94%, 95%, 96%, 97% 98% or 99% sequence identity to SEQ ID NO: 187. In some embodiments, a targeting moiety comprises an amino acid sequence having identical to SEQ ID NO: 187.

In some embodiments, a targeting moiety comprises a light chain CDR1 sequence of QSVSSY (SEQ ID NO: 188), a light chain CDR2 sequence of DAS (SEQ ID NO: 189), a light chain CDR3 sequence of QQSSNWPRT (SEQ ID NO: 190).

In some embodiments, the targeting moiety comprises a heavy chain variable region, and a CH1 domain. In some embodiments, the heavy chain variable region and the CH1 domain comprises the amino acid sequence (SEQ ID NO: 185)

(SEQ ID NO: 185)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNH
KPSNTKVDKKVEPKSC.

In some embodiments, a targeting domain is fused to an Fc polypeptide. In some embodiments, the C-terminus of a targeting domain is fused to the N-terminus of an Fc polypeptide. In some embodiments, the heavy chain of a Fab is fused to an Fc polypeptide. In some embodiments, the C-terminus of the heavy chain of a Fab is fused to the N-terminus of an Fc polypeptide. In some embodiments, an Fc polypeptide comprises a cleavage site.

In some embodiments, the targeting moiety comprises a heavy chain variable region, a light chain variable region and a CH1 domain. In some embodiments, the CH1 domain is derived from IgG1, IgG2, IgG3 or IgG4. In some embodiments, the CH1 domain is derived from IgG1. In some embodiments, the CH1 domain is derived from IgG4.

### Exemplary Masked Cytokine Formats

The present invention provides, among other things, a masked cytokine which comprises an IL-2 polypeptide or variant thereof, a masking moiety, and a carrier moiety. Masked IL-2 cytokines of the present invention become active at the tumor site of through tumor specific protease cleavage of a cleavage peptide located within the masked cytokine that releases the IL-2 polypeptide upon cleavage.

In accordance with the present disclosure, the anti-IL-2 VHH antibody described herein can be utilized in any masked IL-2 cytokine. In some embodiments, the masked cytokine comprises a carrier moiety, a masking moiety, and a cytokine. In some embodiments, the masked cytokine comprises a carrier moiety, an IL-2 polypeptide as the cytokine, and an anti-IL-2 VHH antibody as the masking moiety.

In particular, the masked cytokine or targeted cytokine comprising VHH masking moieties of the present invention is characterized with (1) effective masking efficiency such that IL-2 cytokine's function is inhibited in undesired targets; (2) efficient IL-2 activation by protease to release the VHH masking moiety; (3) selective IL-2 activation in tumor, and not in plasma; and (4) in vivo efficacy (e.g., high tumor growth inhibition).

In some embodiments, a masked cytokine comprises an attenuated IL-2 cytokine, a VHH masking moiety, and a carrier moiety. In some embodiments, a masked cytokine comprises an attenuated IL-2 cytokine, a VHH masking moiety, and an Fc domain. In some embodiments, a masked cytokine comprises an attenuated IL-2 cytokine comprising a mutation of F42E, a VHH masking moiety, and a carrier moiety. In some embodiments, a masked cytokine comprises an attenuated IL-2 cytokine comprising mutations of F42E and C125A, a VHH masking moiety, and a carrier moiety. In some embodiments, a masked cytokine comprises an attenuated IL-2 cytokine comprising mutations of F42E and C125A, a VHH masking moiety comprising a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8), and a carrier moiety. In some embodiments, a masked cytokine comprises an attenuated IL-2 cytokine comprising mutations of F42E and C125A, a VHH masking moiety comprising an amino acid sequence of SEQ ID NO: 5, and a carrier moiety. In some embodiments, a masked cytokine comprises an attenuated IL-2 cytokine comprising mutations of F42E and C125A, a VHH masking moiety comprising an amino acid sequence of SEQ ID NO: 12, and a carrier moiety.

In some embodiments, a masked cytokine comprises an attenuated IL-2 cytokine comprising an amino acid sequence of SEQ ID NO: 4, a VHH masking moiety comprising a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8), and a carrier moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 5, and a carrier moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 12, and a carrier moiety.

In some embodiments, a masked cytokine comprises an attenuated IL-2 cytokine comprising an amino acid sequence of SEQ ID NO: 4, a VHH masking moiety comprising a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8), a carrier moiety, and a targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 5, a carrier moiety, and a targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 12, a carrier moiety, and a targeting moiety.

In some embodiments, a masked cytokine comprises an attenuated IL-2 cytokine comprising an amino acid sequence of SEQ ID NO: 4, a VHH masking moiety comprising a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8), a carrier moiety, and an anti-PD1 targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 5, a carrier moiety, and an anti-PD1 targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 12, a carrier moiety, and an anti-PD1 targeting moiety.

In some embodiments, a masked cytokine comprises an attenuated IL-2 cytokine comprising an amino acid sequence of SEQ ID NO: 4, a VHH masking moiety comprising a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8), a carrier moiety, and an anti-PD1 targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 5, a carrier moiety, and an anti-PD1 targeting moiety. In some embodiments, a masked cytokine comprises an attenuated IL-2 cytokine comprising an amino acid sequence of SEQ ID NO: 4, a VHH masking moiety comprising an amino acid sequence of SEQ ID NO: 12, a carrier moiety, and an anti-PD1 targeting moiety.

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine from N- to C-terminus, and wherein the second polypeptide comprises a targeting moiety, a second Fc polypeptide, a second linker, and a VHH masking moiety from N- to C-terminus.

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine comprising a mutation of F42E from N- to C-terminus, and wherein the second polypeptide comprises a targeting moiety, a second Fc polypeptide, a second linker, and a VHH masking moiety from N- to C-terminus.

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine comprising mutation of F42E and C125A from N- to C-terminus, and wherein the second polypeptide comprises a targeting moiety, a second Fc polypeptide, a second linker, and a VHH masking moiety from N- to C-terminus.

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine comprising mutation of F42E and C125A from N- to C-terminus, wherein the second polypeptide comprises a targeting moiety, a second Fc polypeptide, a second linker, and a VHH masking moiety from N- to C-terminus, and wherein the VHH making moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine comprising mutation of F42E and C125A from N- to C-terminus, and wherein the second polypeptide comprises a targeting moiety, a second Fc polypeptide, a second linker, and a VHH masking moiety of SEQ ID NO: 5 from N- to C-terminus.

In one aspect, the present invention provides, among other things, a masked cytokine comprising a first polypeptide and a second polypeptide, wherein the first polypeptide comprises a targeting moiety, a first Fc polypeptide, a first linker, and an attenuated IL-2 cytokine comprising mutation of F42E and C125A from N- to C-terminus, and wherein the second polypeptide comprises a targeting moiety, a second Fc polypeptide, a second linker, and a VHH masking moiety of SEQ ID NO: 12 from N- to C-terminus.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine comprising mutation of F42E and C125A from N- to C-terminus, wherein the second polypeptide comprises a targeting moiety, a second Fc polypeptide comprising Y349C, T366S, L368A, Y407V, and N297A mutations, a second linker, and a VHH masking moiety from N- to C-terminus, and wherein the VHH making moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine comprising mutation of F42E and C125A from N- to C-terminus, and wherein the second polypeptide comprises a targeting moiety, a second Fc polypeptide comprising Y349C, T366S, L368A, Y407V, and N297A mutations, a second linker, and a VHH masking moiety of SEQ ID NO: 5 from N- to C-terminus.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine comprising mutation of F42E and C125A from N- to C-terminus, and wherein the second polypeptide comprises a targeting moiety, a second Fc polypeptide comprising Y349C, T366S, L368A, Y407V, and N297A mutations, a second linker, and a VHH masking moiety of SEQ ID NO: 12 from N- to C-terminus.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, wherein the second polypeptide comprises a targeting moiety, a second Fc polypeptide comprising Y349C, T366S, L368A, Y407V, and N297A mutations, a second linker, and a VHH masking moiety from N- to C-terminus, and wherein the VHH making moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises a targeting moiety, a second Fc polypeptide comprising Y349C, T366S, L368A, Y407V, and N297A mutations, a second linker, and a VHH masking moiety of SEQ ID NO: 5 from N- to C-terminus.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises a targeting moiety, a second Fc polypeptide comprising Y349C, T366S, L368A, Y407V, and N297A mutations, a second linker, and a VHH masking moiety of SEQ ID NO: 12 from N- to C-terminus.

In some embodiments, the second linker is cleavable.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, wherein the second polypeptide comprises a targeting moiety, a second Fc polypeptide comprising Y349C, T366S, L368A, Y407V, and N297A mutations, a second linker comprising a cleavage peptide, and a VHH masking moiety from N- to C-terminus, and wherein the VHH making moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises a targeting moiety, a second Fc polypeptide comprising Y349C, T366S, L368A, Y407V, and N297A mutations, a second linker comprising a cleavage peptide, and a VHH masking moiety of SEQ ID NO: 5 from N- to C-terminus.

In one aspect, the present invention provides, among other things, a masked cytokine comprising a first polypeptide and a second polypeptide, wherein the first polypeptide comprises a targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises a targeting moiety, a second Fc polypeptide comprising Y349C, T366S, L368A, Y407V, and N297A mutations, a second linker comprising a cleavage peptide, and a VHH masking moiety of SEQ ID NO: 12 from N- to C-terminus.

In one aspect, the present invention provides, among other things, a masked cytokine comprising a first polypeptide and a second polypeptide, wherein the first polypeptide comprises an anti-PD1 targeting moiety, a first Fc polypeptide comprising S354C, T366W, N297A, H435R, and Y435F mutations, a first linker, and an attenuated IL-2 cytokine of SEQ ID NO: 4 from N- to C-terminus, wherein the second polypeptide comprises an anti-PD1 targeting moiety, a second Fc polypeptide comprising Y349C, T366S, L368A, Y407V, and N297A mutations, a second linker comprising a cleavage peptide, and a VHH masking moiety from N- to C-terminus, and wherein the VHH making moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide and a second polypeptide, wherein the first polypeptide comprises an anti-PD1 targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises an anti-PD1 targeting moiety, a second Fc polypeptide comprising Y349C, T366S, L368A, Y407V, and N297A mutations, a second linker comprising a cleavage peptide, and a VHH masking moiety of SEQ ID NO: 5 from N- to C-terminus.

In one aspect, the present invention provides, among other things, a masked cytokine comprising a first polypeptide and a second polypeptide, wherein the first polypeptide comprises an anti-PD1 targeting moiety, a first Fc polypeptide comprising S354C, T366W, N297A, H435R, and Y435F mutations, a first linker, and an attenuated IL-2 cytokine of SEQ ID NO: 4 from N- to C-terminus, and wherein the second polypeptide comprises an anti-PD1 targeting moiety, a second Fc polypeptide comprising Y349C, T366S, L368A, Y407V, and N297A mutations, a second linker comprising a cleavage peptide, and a VHH masking moiety of SEQ ID NO: 12 from N- to C-terminus.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine, wherein the second polypeptide comprises an anti-PD1 targeting moiety, a second Fc polypeptide, a linker and a VHH masking moiety, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine comprising F42E and C125A mutations, wherein the second polypeptide comprises an anti-PD1 targeting moiety, a second Fc polypeptide, a linker and a VHH masking moiety, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine comprising F42E and C125A mutations, wherein the second polypeptide comprises an anti-PD1 targeting moiety, a second Fc polypeptide, a linker and a VHH masking moiety, wherein the third and fourth polypeptides comprises an anti-PD1 targeting moiety, and wherein the VHH masking moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine comprising F42E and C125A mutations, wherein the second polypeptide comprises an anti-PD1 targeting moiety, a second Fc polypeptide, a linker and a VHH masking moiety of SEQ ID NO: 5, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine comprising F42E and C125A mutations, wherein the second polypeptide comprises an anti-PD1 targeting moiety, a second Fc polypeptide, a linker and a VHH masking moiety of SEQ ID NO: 12, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine of SEQ ID NO: 4, wherein the second polypeptide comprises an anti-PD1 targeting moiety, a second Fc polypeptide, a linker and a VHH masking moiety, wherein the third and fourth polypeptides comprises an anti-PD1 targeting moiety, and wherein the VHH masking moiety comprises a CDR1 of GSIFSINVMG (SEQ ID NO: 6), a CDR2 of AISSGGSTNYADSVKG (SEQ ID NO: 7), and a CDR3 of ASSWYEDETDY (SEQ ID NO: 8).

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine of SEQ ID NO: 4, wherein the second polypeptide comprises an anti-PD1 targeting moiety, a second Fc polypeptide, a linker and a VHH masking moiety of SEQ ID NO: 5, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety, a first Fc polypeptide, a linker, and an attenuated IL-2 cytokine of SEQ ID NO: 4, wherein the second polypeptide comprises an anti-PD1 targeting moiety, a second Fc polypeptide, a linker and a VHH masking moiety of SEQ ID NO: 12, wherein the third and fourth polypeptides comprises an anti-PD1 targeting moiety.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a linker and a VHH masking moiety of SEQ ID NO: 5, wherein the third and fourth polypeptides each comprises an anti-PD1 targeting moiety of SEQ ID NO: 176.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a linker and a VHH masking moiety of SEQ ID NO: 12, wherein the third and fourth polypeptides each comprises an anti-PD1 targeting moiety of SEQ ID NO: 176.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a non-cleavable linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a cleavable linker and a VHH masking moiety of SEQ ID NO: 5, wherein the third and fourth polypeptides each comprises an anti-PD1 targeting moiety of SEQ ID NO: 176.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a non-cleavable linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a cleavable linker and a VHH masking moiety of SEQ ID NO: 12, wherein the third and fourth polypeptides each comprises an anti-PD1 targeting moiety of SEQ ID NO: 176.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a non-cleavable linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a cleavable linker comprising SEQ ID NO: 34 and a VHH masking moiety of SEQ ID NO: 5, wherein the third and fourth polypeptides each comprises an anti-PD1 targeting moiety of SEQ ID NO: 176.

In one aspect, the present invention provides, among other things, a masked cytokine comprising a first polypeptide, a second polypeptide, a third polypeptide, and a fourth polypeptide, wherein the first polypeptide comprises an anti-PD1 targeting moiety of SEQ ID NO: 170, a first Fc polypeptide of SEQ ID NO: 21, a non-cleavable linker, and an attenuated IL-2 cytokine of SEQ ID NO: 4, wherein the second polypeptide comprises an anti-PD1 targeting moiety of SEQ ID NO: 170, a second Fc polypeptide of SEQ ID NO: 20, a cleavable linker comprising SEQ ID NO: 34 and a VHH masking moiety of SEQ ID NO: 12, wherein the third and fourth polypeptides each comprises an anti-PD1 targeting moiety of SEQ ID NO: 176.

In some embodiments, a masked cytokine or a targeted cytokine has an EC50 value based on pSTAT5 activity on CD8+ T cells of greater than 30-fold as compared to an IL-2 cytokine without a masking moiety. In some embodiments, a masked cytokine or a targeted cytokine has an EC50 value based on pSTAT5 activity on CD8+ T cells of greater than 50-fold as compared to an IL-2 cytokine without a masking moiety. In some embodiments, a masked cytokine or a targeted cytokine has an EC50 value based on pSTAT5 activity on CD8+ T cells of greater than 100-fold as compared to an IL-2 cytokine without a masking moiety. In some embodiments, a masked cytokine or a targeted cytokine has an EC50 value based on pSTAT5 activity on CD8+ T cells of greater than 200-fold as compared to an IL-2 cytokine without a masking moiety. In some embodiments, a masked cytokine or a targeted cytokine has an EC50 value based on pSTAT5 activity on CD8+ T cells of greater than 300-fold as compared to an IL-2 cytokine without a masking moiety. In some embodiments, a masked cytokine or a targeted cytokine has an EC50 value based on pSTAT5 activity on CD8+ T cells of greater than 500-fold as compared to an IL-2 cytokine without a masking moiety. In some embodiments, a masked cytokine or a targeted cytokine has an EC50 value based on pSTAT5 activity on CD8+ T cells of greater than 700-fold as compared to an IL-2 cytokine without a masking moiety. In some embodiments, a masked cytokine or a targeted cytokine has an EC50 value based on pSTAT5 activity on CD8+ T cells of greater than 800-fold as compared to an IL-2 cytokine without a masking moiety. In some embodiments, a masked cytokine or a targeted cytokine has an EC50 value based on pSTAT5 activity on CD8+ T cells of greater than 1000-fold as compared to an IL-2 cytokine without a masking moiety. In some embodiments, a masked cytokine or a targeted cytokine has an EC50 value based on pSTAT5 activity on CD8+ T cells of greater than 3000-fold as compared to an IL-2 cytokine without a masking moiety. In some embodiments, a masked cytokine or a targeted cytokine has an EC50 value based on pSTAT5 activity on CD8+ T cells of greater than 5000-fold as compared to an IL-2 cytokine without a masking moiety.

In some embodiments, a masked cytokine or a targeted cytokine is cleaved by at least one MMP with a Kcat/Km of greater than 10⁴ M⁻¹s⁻¹. In some embodiments, a masked cytokine or a targeted cytokine is cleaved by at least two MMPs with a Kcat/Km of greater than 10⁴ M⁻¹s⁻¹. In some embodiments, a masked cytokine or a targeted cytokine is cleaved by at least three MMPs with a Kcat/Km of greater than 10⁴ M⁻¹s⁻¹. In some embodiments, a masked cytokine or a targeted cytokine is cleaved by at least four MMPs with a Kcat/Km of greater than 10⁴ M⁻¹s⁻¹. In some embodiments, a masked cytokine or a targeted cytokine is cleaved by at least five MMPs with a Kcat/Km of greater than 10⁴ M⁻¹s⁻¹. In some embodiments, a masked cytokine or a targeted cytokine is cleaved by at least one MMP with a Kcat/Km of greater than 5×10⁴ M⁻¹s⁻¹. In some embodiments, a masked cytokine or a targeted cytokine is cleaved by at least two MMPs with a Kcat/Km of greater than 5×10⁴ M⁻¹s⁻¹. In some embodiments, a masked cytokine or a targeted cytokine is cleaved by at least three MMPs with a Kcat/Km of greater than 5×10⁴ M⁻¹s⁻¹. In some embodiments, a masked cytokine or a targeted cytokine is cleaved by at least four MMPs with a Kcat/Km of greater than 5×10⁴ M⁻¹s⁻¹. In some embodiments, a masked cytokine or a targeted cytokine is cleaved by at least five MMPs with a Kcat/Km of greater than 5×10⁴ M⁻¹s⁻¹. In some embodiments, a masked cytokine or a targeted cytokine is cleaved by at least one MMP with a Kcat/Km of greater than 10⁵ M⁻¹s⁻¹. In some embodiments, a masked cytokine or a targeted cytokine is cleaved by at least two MMPs with a Kcat/Km of greater than 10⁵ M⁻¹s⁻¹. In some embodiments, a masked cytokine or a targeted cytokine is cleaved by at least three MMPs with a Kcat/Km of greater than 10⁵ M⁻¹s⁻¹. In some embodiments, a masked cytokine or a targeted cytokine is cleaved by at least four MMPs with a Kcat/Km of greater than 10⁵ M⁻¹s⁻¹. In some embodiments, a masked cytokine or a targeted cytokine is cleaved by at least five MMPs with a Kcat/Km of greater than 10⁵ M⁻¹s⁻¹.

In some embodiments, a masked cytokine or a targeted cytokine, after protease cleavage, has an EC50 value based on pSTAT5 activity on CD8+ T cells within 5-fold as compared to an IL-2 cytokine without a masking moiety. In some embodiments, a masked cytokine or a targeted cytokine, after protease cleavage, has an EC50 value based on pSTAT5 activity on CD8+ T cells within 4-fold as compared to an IL-2 cytokine without a masking moiety. In some embodiments, a masked cytokine or a targeted cytokine, after protease cleavage, has an EC50 value based on pSTAT5 activity on CD8+ T cells within 3-fold as compared to an IL-2 cytokine without a masking moiety. In some embodiments, a masked cytokine or a targeted cytokine, after protease cleavage, has an EC50 value based on pSTAT5 activity on CD8+ T cells within 2-fold as compared to an IL-2 cytokine without a masking moiety. In some embodiments, a masked cytokine or a targeted cytokine, after protease cleavage, has an EC50 value based on pSTAT5 activity on CD8+ T cells within 1-fold as compared to an IL-2 cytokine without a masking moiety. In some embodiments, a masked cytokine or a targeted cytokine, after protease cleavage, has an EC50 value based on pSTAT5 activity on CD8+ T cells within 0.8-fold as compared to an IL-2 cytokine without a masking moiety.

In some embodiments, % cleaved molecules of a masked cytokine or a targeted cytokine in in tumor is at least 2%. In some embodiments, % cleaved molecules of a masked cytokine or a targeted cytokine in in tumor is at least 3%. In some embodiments, % cleaved molecules of a masked cytokine or a targeted cytokine in in tumor is at least 4%. In some embodiments, % cleaved molecules of a masked cytokine or a targeted cytokine in in tumor is at least 2%. In some embodiments, % cleaved molecules of a masked cytokine or a targeted cytokine in in tumor is at least 5%. In some embodiments, % cleaved molecules of a masked cytokine or a targeted cytokine in in tumor is at least 7%. In some embodiments, % cleaved molecules of a masked cytokine or a targeted cytokine in in tumor is at least 8%. In some embodiments, % cleaved molecules of a masked cytokine or a targeted cytokine in in tumor is at least 10%. In some embodiments, % cleaved molecules of a masked cytokine or a targeted cytokine in in tumor is at least 12%. In some embodiments, % cleaved molecules of a masked cytokine or a targeted cytokine in in tumor is at least 14%. In some embodiments, % cleaved molecules of a masked cytokine or a targeted cytokine in in tumor is at least 15%. In some embodiments, % cleaved molecules of a masked cytokine or a targeted cytokine in in tumor is at least 20%. In some embodiments, % cleaved molecules of a masked cytokine or a targeted cytokine in in tumor is at least 30%. In some embodiments, % cleaved molecules of a masked cytokine or a targeted cytokine in in tumor is at least 40%. In some embodiments, % cleaved molecules of a masked cytokine or a targeted cytokine in in tumor is at least 50%. In some embodiments, % cleaved molecules of a masked cytokine or a targeted cytokine in in tumor is at least 55%. In some embodiments, % cleaved molecules of a masked cytokine or a targeted cytokine in in tumor is at least 60%. In some embodiments, % cleaved molecules of a masked cytokine or a targeted cytokine in in tumor is below the lower limit of quantitation (LLOQ) in plasma.

In some embodiments, a masked cytokine or a targeted cytokine has tumor growth inhibition (TGI) of at least 40%. In some embodiments, a masked cytokine or a targeted cytokine has tumor growth inhibition (TGI) of at least 50%. In some embodiments, a masked cytokine or a targeted cytokine has tumor growth inhibition (TGI) of at least 55%. In some embodiments, a masked cytokine or a targeted cytokine has tumor growth inhibition (TGI) of at least 60%. In some embodiments, a masked cytokine or a targeted cytokine has tumor growth inhibition (TGI) of at least 65%. In some embodiments, a masked cytokine or a targeted cytokine has tumor growth inhibition (TGI) of at least 70%. In some embodiments, a masked cytokine or a targeted cytokine has tumor growth inhibition (TGI) of at least 75%. In some embodiments, a masked cytokine or a targeted cytokine has tumor growth inhibition (TGI) of at least 80%. In some embodiments, a masked cytokine or a targeted cytokine has tumor growth inhibition (TGI) of at least 85%. In some embodiments, a masked cytokine or a targeted cytokine has tumor growth inhibition (TGI) of at least 90%. In some embodiments, a masked cytokine or a targeted cytokine has tumor growth inhibition (TGI) of at least 95%.

### Masked IL-2 Cytokine

In some embodiments, a masked cytokine of the present invention comprises a masking moiety, a carrier moiety, and a cytokine or variant thereof. In some embodiments, a masked cytokine comprises a masking moiety, an Fc domain as a carrier moiety, and a cytokine or variant thereof. In some embodiments, a masked cytokine comprises a VHH antibody as a masking moiety, a carrier moiety, and a cytokine or variant thereof. In some embodiments, a masked cytokine comprises a VHH antibody as a masking moiety, an Fc domain as a carrier moiety, and a cytokine or variant thereof.

In some embodiments, the IL-2 polypeptide is linked to an Fc domain via a cleavable or a non-cleavable linker.

In some embodiments, the masking moiety is linked to an Fc domain via a cleavable or a non-cleavable linker. In some embodiments, the targeting moiety is linked to an Fc domain with or without cleavable or a non-cleavable linker. In some embodiments, the masking moiety is linked to an Fc polypeptide via a cleavable linker. In some embodiments, the masking moiety is linked to an Fc polypeptide via a non-cleavable linker. In some embodiments, the IL-2 polypeptide is linked to an Fc polypeptide via a cleavable linker. In some embodiments, the IL-2 polypeptide is linked to an Fc polypeptide via a non-cleavable linker.

In some embodiments, the first polypeptide comprises SEQ ID NO: 191. In some embodiments, the second polypeptide comprises SEQ ID NO: 192. In some embodiments, the third polypeptide comprises SEQ ID NO: 176. In some embodiments, the first polypeptide comprises SEQ ID NO: 191, the second polypeptide comprises SEQ ID NO: 192; and the third polypeptide comprises SEQ ID NO: 176.

In some embodiments, a masked cytokine comprises three polypeptides: (a) a first polypeptide comprising an amino sequence that is at least 90%, at least 95%, at least 96%, at least 97%, at least 98%, or at least 99% identical to SEQ ID NO: 191, (b) a second polypeptide comprising an amino sequence that is at least 90%, at least 95%, at least 96%, at least 97%, at least 98%, or at least 99% identical to SEQ ID NO: 193, and (c) a third polypeptide comprising an amino sequence that is at least 90%, at least 95%, at least 96%, at least 97%, at least 98%, or at least 99% identical to SEQ ID NO: 176.

In some embodiments, the first polypeptide comprises an amino acid sequence that is at least 90% identical to SEQ ID NO: 191. In some embodiments, the first polypeptide comprises an amino acid sequence that is at least 92% identical to SEQ ID NO: 191. In some embodiments, the first polypeptide comprises an amino acid sequence that is at least 95% identical to SEQ ID NO: 191. In some embodiments, the first polypeptide comprises an amino acid sequence that is at least 96% identical to SEQ ID NO: 191. In some embodiments, the first polypeptide comprises an amino acid sequence that is at least 97% identical to SEQ ID NO: 191. In some embodiments, the first polypeptide comprises an amino acid sequence that is at least 98% identical to SEQ ID NO: 191. In some embodiments, the first polypeptide comprises an amino acid sequence that is at least 99% identical to SEQ ID NO: 191.

In some embodiments, the second polypeptide comprises an amino acid sequence that is at least 90% identical to SEQ ID NO: 193. In some embodiments, the second polypeptide comprises an amino acid sequence that is at least 92% identical to SEQ ID NO: 193. In some embodiments, the second polypeptide comprises an amino acid sequence that is at least 95% identical to SEQ ID NO: 193. In some embodiments, the second polypeptide comprises an amino acid sequence that is at least 96% identical to SEQ ID NO: 193. In some embodiments, the second polypeptide comprises an amino acid sequence that is at least 97% identical to SEQ ID NO: 193. In some embodiments, the second polypeptide comprises an amino acid sequence that is at least 98% identical to SEQ ID NO: 193. In some embodiments, the second polypeptide comprises an amino acid sequence that is at least 99% identical to SEQ ID NO: 193.

In some embodiments, the third polypeptide comprises an amino acid sequence that is at least 90% identical to SEQ ID NO: 176. In some embodiments, the third polypeptide comprises an amino acid sequence that is at least 92% identical to SEQ ID NO: 176. In some embodiments, the third polypeptide comprises an amino acid sequence that is at least 95% identical to SEQ ID NO: 176. In some embodiments, the third polypeptide comprises an amino acid sequence that is at least 96% identical to SEQ ID NO: 176. In some embodiments, the third polypeptide comprises an amino acid sequence that is at least 97% identical to SEQ ID NO: 176. In some embodiments, the third polypeptide comprises an amino acid sequence that is at least 98% identical to SEQ ID NO: 176. In some embodiments, the third polypeptide comprises an amino acid sequence that is at least 99% identical to SEQ ID NO: 176.

In some embodiments, a masked cytokine comprises three polypeptides: (a) a first polypeptide comprising an amino sequence of SEQ ID NO: 191, (b) a second polypeptide comprising an amino sequence of SEQ ID NO: 193, and (c) a third polypeptide comprising an amino sequence of SEQ ID NO: 176.

TABLE 13
Exemplary sequences of Masked Cytokines.

| SEQ ID | |
|---|---|
| NO | Sequence |
| 191 | QVQLVQSGVEVKKPGASVKVSCKASGYTFTNYYMYWVRQAPGQGLEWMGGINPSNGGTNEN |
| | EKFKNRVTLTTDSSTTTAYMELKSLQFDDTAVYYCARRDYRFDMGFDYWGQGTTVTVSSAS |
| | TKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPPCPAPELLGGPSVE |
| | LFPPKPKDTLMISRTPEVTCVVVDVSHEDPEVKENWYVDGVEVHNAKTKPREEQYASTYRV |
| | VSVLTVLHQDWLNGKEYKCKVSNKALPAPIEKTISKAKGQPREPQVYTLPPCRDELTKNQV |
| | SLWCLVKGFYPSDIAVEWESNGQPENNYKTTPPVLDSDGSFFLYSKLTVDKSRWQQGNVES |
| | CSVMHEALHNRFTQKSLSLSPGGGSSPPGGGSSGGGSGPAPTSSSTKKTQLQLEHLLLDLQ |
| | MILNGINNYKNPKLTRMLTEKFYMPKKATELKHLQCLEEELKPLEEVLNLAQSKNFHLRPR |
| | DLISNINVIVLELKGSETTEMCEYADETATIVEFLNRWITFAQSIISTLT |
| 192 | QVQLVQSGVEVKKPGASVKVSCKASGYTFTNYYMYWVRQAPGQGLEWMGGINPSNGGTNEN |
| | EKFKNRVTLTTDSSTTTAYMELKSLQFDDTAVYYCARRDYRFDMGEDYWGQGTTVTVSSAS |
| | TKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPPCPAPELLGGPSVE |
| | LFPPKPKDTLMISRTPEVTCVVVDVSHEDPEVKENWYVDGVEVHNAKTKPREEQYASTYRV |
| | VSVLTVLHQDWLNGKEYKCKVSNKALPAPIEKTISKAKGQPREPQVCTLPPSRDELTKNQV |
| | SLSCAVKGFYPSDIAVEWESNGQPENNYKTTPPVLDSDGSFFLVSKLTVDKSRWQQGNVES |
| | CSVMHEALHNHYTQKSLSLSPGSPGGGGPMPYDLYHPSGGGEVQLVESGGGLVQPGGSLRL |
| | SCAASGSIFSINVMGWYRQAPGKQRELVAAISSGGSTNYADSVKGRFTISRDNAKNTVYLQ |
| | MNSLKPEDTAVYYCMYASSWYEDETDYWGQGTQVTVSSAAA |
| 193 | QVQLVQSGVEVKKPGASVKVSCKASGYTFTNYYMYWVRQAPGQGLEWMGGINPSNGGTNEN |
| | EKFKNRVTLTTDSSTTTAYMELKSLQFDDTAVYYCARRDYRFDMGFDYWGQGTTVTVSSAS |
| | TKGPSVFPLAPSSKSTSGGTAALGCLVKDYFPEPVTVSWNSGALTSGVHTFPAVLQSSGLY |
| | SLSSVVTVPSSSLGTQTYICNVNHKPSNTKVDKKVEPKSCDKTHTCPPCPAPELLGGPSVE |
| | LFPPKPKDTLMISRTPEVTCVVVDVSHEDPEVKENWYVDGVEVHNAKTKPREEQYASTYRV |
| | VSVLTVLHQDWLNGKEYKCKVSNKALPAPIEKTISKAKGQPREPQVCTLPPSRDELTKNQV |
| | SLSCAVKGFYPSDIAVEWESNGQPENNYKTTPPVLDSDGSFFLVSKLTVDKSRWQQGNVES |
| | CSVMHEALHNHYTQKSLSLSPGSPGGGGPMPYDLYHPSGGGEVQLVESGGGLVQPGGSLRL |
| | SCAASGSIFSINVMGWYRQAPGKQRELVAAISSGGSTNYADSVKGRFTISRDNAKNTVYLQ |
| | MNSLKPEDTAVYYCMYASSWYEDETDYWGQGTQVTVSS |

### Targeted IL-2 Cytokine

In some embodiments, a masked IL-2 cytokine further comprises a targeting moiety to form a targeted IL-2 cytokine.

In some embodiments, a targeted cytokine of the present invention comprises a masking moiety, a carrier moiety, a targeting moiety and a cytokine or variant thereof. In some embodiments, a targeted cytokine comprises a masking moiety, an Fc domain as a carrier moiety, a targeting moiety and a cytokine or variant thereof. In some embodiments, a targeted cytokine comprises a VHH antibody as a masking moiety, a carrier moiety, a targeting moiety and a cytokine or variant thereof. In some embodiments, a targeted cytokine comprises a VHH antibody as a masking moiety, an Fc domain as a carrier moiety, a targeting moiety and a cytokine or variant thereof.

In some embodiments, the IL-2 polypeptide is linked to an Fc domain via a cleavable or a non-cleavable linker.

In some embodiments, the masking moiety is linked to an Fc domain via a cleavable or a non-cleavable linker. In some embodiments, the targeting moiety is linked to an Fc domain with or without cleavable or a non-cleavable linker. In some embodiments, the masking moiety is linked to an Fc polypeptide via a cleavable linker. In some embodiments, the masking moiety is linked to an Fc polypeptide via a non-cleavable linker. In some embodiments, the IL-2 polypeptide is linked to an Fc polypeptide via a cleavable linker. In some embodiments, the IL-2 polypeptide is linked to an Fc polypeptide via a non-cleavable linker.

In some embodiments, a targeted cytokine comprises a first polypeptide comprising a heavy chain of a targeting moiety and a first Fc polypeptide linked to a cytokine via a non-cleavable linker in N-to-C terminus orientation, a second polypeptide comprising a heavy chain of the targeting moiety, a second Fc polypeptide linked to a masking moiety via a cleavable linker in N-to-C terminus orientation, and a third polypeptide comprising a light chain of the targeting moiety that associates with the heavy chain of the targeting moiety.

In some embodiments, a targeted cytokine comprises a first polypeptide comprising a heavy chain of a targeting moiety and a first Fc polypeptide linked to a cytokine via a cleavable linker in N-to-C terminus orientation, a second polypeptide comprising a heavy chain of the targeting moiety, a second Fc polypeptide linked to a masking moiety via a non-cleavable linker in N-to-C terminus orientation, and a third polypeptide comprising a light chain of the targeting moiety that associates with the heavy chain of the targeting moiety.

In some embodiments, a targeted cytokine comprises a first polypeptide comprising a scFv targeting moiety (e.g., heavy chain variable region fused to a light chain variable region (VH-VL or VL-VH)), a first Fc polypeptide linked to a cytokine via a non-cleavable linker in N-to-C terminus orientation, and a second polypeptide comprising a scFv targeting moiety (e.g., heavy chain variable region fused to a light chain variable region (VH-VL or VL-VH)), a second Fc polypeptide linked to a masking moiety via a cleavable linker in N-to-C terminus orientation.

In some embodiments, a targeted cytokine comprises a first polypeptide comprising a scFv targeting moiety (e.g., heavy chain variable region fused to a light chain variable region (VH-VL or VL-VH)), a first Fc polypeptide linked to a cytokine via a cleavable linker in N-to-C terminus orientation, and a second polypeptide comprising a scFv targeting moiety (e.g., heavy chain variable region fused to a light chain variable region (VH-VL or VL-VH)), a second Fc polypeptide linked to a masking moiety via a non-cleavable linker in N-to-C terminus orientation.

### Methods of Treatment

Provided herein are methods for treating or preventing a disease in a subject comprising administering to the subject an effective amount of any masked and/or targeted cytokine described herein or compositions thereof. In some embodiments, methods are provided for treating or preventing a disease in a subject comprising administering to the subject any composition described herein. In some embodiments, the subject (e.g., a human patient) has been diagnosed with cancer or is at risk of developing such a disorder. In some embodiments, methods are provided for treating or preventing disease in a subject comprising administering to the subject an effective amount of any targeted cytokine described herein or compositions thereof, wherein the targeted cytokine is activated upon cleavage by an enzyme. In some embodiments, the targeted cytokine is activated at a tumor microenvironment. The targeted cytokine is therapeutically active after it has cleaved. Thus, in some embodiments, the active agent is the cleavage product. In some embodiments, the targeted masked cytokine is therapeutically active after the masking moiety has been cleaved. Thus, in some embodiments, the active agent is the unmasked targeted cytokine.

In some embodiments, the masked cytokine is activated at a tumor microenvironment. The masked cytokine is therapeutically active after the masking moiety has been cleaved. Thus, in some embodiments, the active agent is the unmasked cytokine. In some embodiments, the masked cytokine is therapeutically active after the cytokine has been cleaved. Thus, in some embodiments, the active agent is the cleavage product.

In one aspect, the present invention provides a method of treating a disorder or disease such as cancer with any of the masked cytokines or compositions described herein. Disorders or diseases that are treatable with the formulations of this present invention include leukemia, lymphoma, head and neck cancer, colorectal cancer, prostate cancer, pancreatic cancer, melanoma, breast cancer, neuroblastoma, lung cancer, ovarian cancer, osteosarcoma, bladder cancer, cervical cancer, liver cancer, kidney cancer, skin cancer (e.g., Merkel cell carcinoma) or testicular cancer.

In some embodiments, provided herein is a method of treating or preventing a cancer by administration of any masked cytokines or compositions described herein. In some embodiments, provided herein is a method of treating or preventing a cancer by administration of any masked cytokine or composition described herein in combination with an anti-cancer agent. The anti-cancer agent can be any agent capable of reducing cancer growth, interfering with cancer cell replication, directly or indirectly killing cancer cells, reducing metastasis, reducing tumor blood supply, or reducing cell survival. In some embodiments, the anti-cancer agent is selected from the group consisting of a PD-1 inhibitor, an EGFR inhibitor, a HER2 inhibitor, a VEGFR inhibitor, a CTLA-4 inhibitor, a BTLA inhibitor, a B7H4 inhibitor, a B7H3 inhibitor, a CSFIR inhibitor, an HVEM inhibitor, a CD27 inhibitor, a KIR inhibitor, an NKG2A inhibitor, an NKG2D agonist, a TWEAK inhibitor, an ALK inhibitor, a CD52 targeting antibody, a CCR4 targeting antibody, a PD-L1 inhibitor, a KIT inhibitor, a PDGFR inhibitor, a BAFF inhibitor, an HD AC inhibitor, a VEGF ligand inhibitor, a CD19 targeting molecule, a FOFR1 targeting molecule, a DFF3 targeting molecule, a DKK1 targeting molecule, a MUC1 targeting molecule, a MUG 16 targeting molecule, a PSMA targeting molecule, an MSFN targeting molecule, an NY-ESO-1 targeting molecule, a B7H3 targeting molecule, a B7H4 targeting molecule, a BCMA targeting molecule, a CD29 targeting molecule, a CD151 targeting molecule, a CD 123 targeting molecule, a CD33 targeting molecule, a CD37 targeting molecule, a CDH19 targeting molecule, a CEA targeting molecule, a Claudin 18.2 targeting molecule, a CFECi2A targeting molecule, an EGFRVIII targeting molecule, an EPCAM targeting molecule, an EPHA2 targeting molecule, an FCRH5 targeting molecule, an FLT3 targeting molecule, a GD2 targeting molecule, a glypican 3 targeting molecule, a gpA33 targeting molecule, a GPRC5D targeting molecule, an IL-23R targeting molecule, an IL-1RAP targeting molecule, a MCSP targeting molecule, a RON targeting molecule, a ROR1 targeting molecule, a STEAP2 targeting molecule, a TfR targeting molecule, a CD166 targeting molecule, a TPBG targeting molecule, a TROP2 targeting molecule, a proteasome inhibitor, an ABE inhibitor, a CD30 inhibitor, a FLT3 inhibitor, a MET inhibitor, a RET inhibitor, an IL-1(3 inhibitor, a MEK inhibitor, a ROS1 inhibitor, a BRAE inhibitor, a CD38 inhibitor, a RANKE inhibitor, a B4GALNT1 inhibitor, a SLAMF7 inhibitor, an IDH2 inhibitor, an mTOR inhibitor, a CD20 targeting antibody, a BTK inhibitor, a PI3K inhibitor, a FLT3 inhibitor, a PARP inhibitor, a CDK4 inhibitor, a CDK6 inhibitor, an EGFR inhibitor, a RAF inhibitor, a JAK1 inhibitor, a JAK2 inhibitor, a JAK3 inhibitor, an IL-6 inhibitor, a IL-17 inhibitor, a Smoothened inhibitor, an IL-6R inhibitor, a BCL2 inhibitor, a PTCH inhibitor, a PIGF inhibitor, a TGFB inhibitor, a CD28 agonist, a CD3 agonist, CD40 agonist, a GITR agonist, a 0X40 agonist, a VISTA agonist, a CD137 agonist, a LAG3 inhibitor, a TIM3 inhibitor, a TIGIT inhibitor, and an IL-2R inhibitor.

In some embodiments, provided herein is a method of treating or preventing a cancer by administration of any masked cytokine described herein in combination with an anti-inflammatory agent. The anti-inflammatory agent can be any agent capable of preventing, counteracting, inhibiting, or otherwise reducing inflammation.

In some embodiments, the anti-inflammatory agent is a cyclooxygenase (COX) inhibitor. The COX inhibitor can be any agent that inhibits the activity of COX-1 and/or COX-2. In some embodiments, the COX inhibitor selectively inhibits COX-1 (i.e., the COX inhibitor inhibits the activity of COX-1 more than it inhibits the activity of COX-2). In some embodiments, the COX inhibitor selectively inhibits COX-2 (i.e., the COX inhibitor inhibits the activity of COX-2 more than it inhibits the activity of COX-1). In some embodiments, the COX inhibitor inhibits both COX-1 and COX-2.

In some embodiments, the COX inhibitor is a selective COX-1 inhibitor and is selected from the group consisting of SC-560, FR122047, P6, mofezolac, TFAP, flurbiprofen, and ketoprofen. In some embodiments, the COX inhibitor is a selective COX-2 inhibitor and is selected from the group consisting of celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, and SD-8381. In some embodiments, the COX inhibitor is selected from the group consisting of ibuprofen, naproxen, ketorolac, indomethacin, aspirin, naproxen, tolmetin, piroxicam, and meclofenamate. In some embodiments, the COX inhibitor is selected from the group consisting of SC-560, FR122047, P6, mofezolac, TFAP, flurbiprofen, ketoprofen, celecoxib, rofecoxib, meloxicam, piroxicam, deracoxib, parecoxib, valdecoxib, etoricoxib, a chromene derivative, a chroman derivative, N-(2-cyclohexyloxynitrophenyl) methane sulfonamide, parecoxib, lumiracoxib, RS 57067, T-614, BMS-347070, JTE-522, S-2474, SVT-2016, CT-3, ABT-963, SC-58125, nimesulide, flosulide, NS-398, L-745337, RWJ-63556, L-784512, darbufelone, CS-502, LAS-34475, LAS-34555, S-33516, diclofenac, mefenamic acid, SD-8381, ibuprofen, naproxen, ketorolac, indomethacin, aspirin, naproxen, tolmetin, piroxicam, and meclofenamate.

In some embodiments, the anti-inflammatory agent is an NF-KB inhibitor. The NF-KB inhibitor can be any agent that inhibits the activity of the NF-KB pathway. In some embodiments, the NF-KB inhibitor is selected from the group consisting of an IKK complex inhibitor, an IKB degradation inhibitor, an NF-KB nuclear translocation inhibitor, a p65 acetylation inhibitor, an NF-KB DNA binding inhibitor, an NF-KB transactivation inhibitor, and a p53 induction inhibitor.

In some embodiments, the IKK complex inhibitor is selected from the group consisting of TPCA-1, NF-KB Activation Inhibitor VI (BOT-64), BMS-345541, amlexanox, SC-514 (GK-01140), IMD-0354, and IKK-16. In some embodiments, the IKB degradation inhibitor is selected from the group consisting of BAY-11-7082, MG-115, MG-132, lactacystin, epoxomicin, parthenolide, carfilzomib, and MLN-4924 (pevonedistat). In some embodiments, the NF-KB nuclear translocation inhibitor is selected from the group consisting of JSH-23 and rolipram. In some embodiments, the p65 acetylation inhibitor is selected from the group consisting of gallic acid and anacardic acid. In some embodiments, the NF-KB DNA binding inhibitor is selected from the group consisting of GYY-4137, p-XSC, CV-3988, and prostaglandin E2 (PGE2). In some embodiments, the NF-KB transactivation inhibitor is selected from the group consisting of LY-294002, wortmannin, and mesalamine. In some embodiments, the p53 induction inhibitor is selected from the group consisting of quinacrine and flavopiridol. In some embodiments, the NF-KB inhibitor is selected from the group consisting of TPCA-1, NF-KB Activation Inhibitor VI (BOT-64), BMS-345541, amlexanox, SC-514 (GK-01140), IMD-0354, IKK-16, BAY-11-7082, MG-115, MG-132, lactacystin, epoxomicin, parthenolide, carfilzomib, MLN-4924 (pevonedistat), JSH-23 rolipram, gallic acid, anacardic acid, GYY-4137, p-XSC, CV-3988, prostaglandin E2 (PGE2), LY-294002, wortmannin, mesalamine, quinacrine, and flavopiridol.

In some embodiments, provided herein is a method of treating or preventing a cancer by administration of any masked cytokine or composition described herein in combination with an anti-cancer therapeutic protein. The anti-cancer therapeutic protein can be any therapeutic protein capable of reducing cancer growth, interfering with cancer cell replication, directly or indirectly killing cancer cells, reducing metastasis, reducing tumor blood supply, or reducing cell survival. Exemplary anti-cancer therapeutic proteins may come in the form of an antibody or fragment thereof, an antibody derivative, a bispecific antibody, a chimeric antigen receptor (CAR) T cell, a fusion protein, or a bispecific T-cell engager (BiTE). In some embodiments, provided herein is a method of treating or preventing a cancer by administration of any masked cytokine or composition described herein in combination with CAR-NK (Natural Killer) cells.

### Methods for Producing Masked Cytokine Constructs

Some embodiments of the methods and compositions provided herein relate to methods for producing a targeted masked agent such as a targeted masked cytokine. The targeted masked cytokines as described herein can be produced and manufactured using any known technology in the art. The targeted masked cytokines and/or constructs may be produced using any mammalian expression system.

Some embodiments of the methods and compositions provided herein relate to methods for producing a masked agent such as a masked cytokine. The masked cytokines as described herein can be produced and manufactured using any known technology in the art. The masked cytokines and/or constructs may be produced using any mammalian expression system.

Mammalian cells are commonly used for producing recombinant proteins. The cells are be derived from human, rat, mouse and other mammals. Commonly used mammalian cell lines include but are not limited to, HEK cells, CHO cells, recombinant CHO cells, BHK cells, NSO cells, SP2/O—Ag14 cells, HT-1080 cells, PER.C6 cells, CAP (CEVEC's Amniocyte Production), Hela cells, and HuH-7 (Human hepatoma) cells. The cell line can be used for transient gene expression or as stable cell line for stable expression.

In some embodiments, the cells are Chinese Hamster ovary (CHO) cells. The CHO cells can be recombinant CHO cell lines, e.g., CHO-K1, CHO DUX and CHO DG44.

In some embodiments, the cells are human cells. In some embodiments, the cells are Human Embryo Kidney (HEK) cells or variants thereof (e.g., HKB11).

The host cells are transformed to express a nucleic acid or vector encoding a targeted masked cytokine described herein. Where appropriate, the engineered host cells can be cultured in conventional nutrient media.

In other embodiments, the targeted masked cytokines may be produced using non-mammalian expression systems, such as baculovirus expression systems, bacterial systems, yeast cells, insect cell lines and plant cells. The insect cell lines include but are not limited to Sf9, Sf21 and BTI 5B1-4. E. coli cells are commonly used bacteria to express a recombinant protein.

In some embodiments, the masked cytokines and/or constructs may be produced using a cell-free protein express system. Cell-free expression systems can use mRNA transcribed from a DNA construct comprising a promoter operably linked to a nucleic acid encoding the polypeptide or fragment thereof.

### EXAMPLES

While certain compounds, compositions and methods of the present invention have been described with specificity in accordance with certain embodiments, the following examples serve only to illustrate the compounds of the invention and are not intended to limit the same.

### Example 1. Effective Masking and Activation of Masked Cytokines

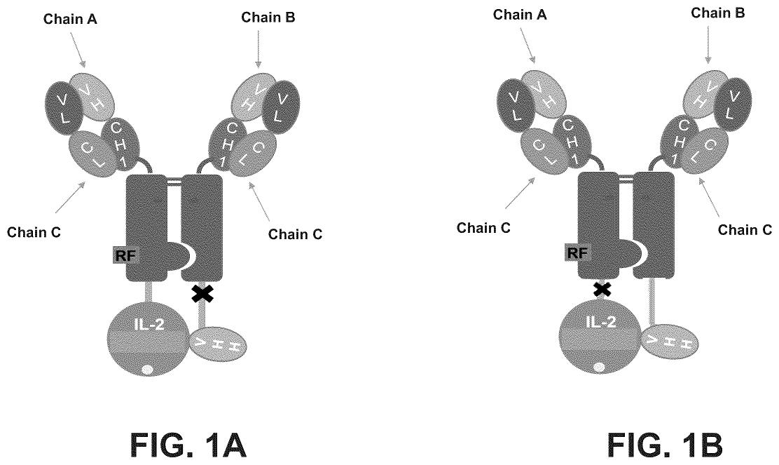

This example demonstrates that the masked IL-2 cytokines of the present invention are effectively masked by the VHH masking moieties, and activated once the VHH masking moieties are released by tumor-specific proteases. In some embodiments, a masked cytokine comprises a format illustrated in FIG. 1A. In some embodiments, a masked cytokine comprises a format illustrated in FIG. 1B.

TABLE 7
Exemplary Masked IL-2 Cytokines

| | | Masking | Targeting | Carrier | |
|---|---|---|---|---|---|
| Construct | Cytokine | Moiety | Moiety | Moiety | Linker |
| C5 | IL-2 V3 | N/A | Anti-PD1 | Fc Domain | Non-Cleavable |
| | N/A | VHH46 | Anti-PD1 | Fc Domain | Cleavable |
| C6 | IL-2 V3 | N/A | Anti-PD1 | Fc Domain | Non-Cleavable |
| | N/A | VHH1 | Anti-PD1 | Fc Domain | Cleavable |
| Unmasked | IL-2 V3 | N/A | Anti-PD1 | Fc Domain | Non-Cleavable |
| Control | N/A | N/A | Anti-PD1 | Fc Domain | N/A |

In this particular example, masked IL-2 cytokines shown in Table 7 were prepared with purity of greater than at least 97%. All masked IL-2 cytokines tested were able to inhibit PD-1/PD-L1 interaction and were efficiently cleaved by at least one MMP (data not shown).

pSTAT5 activity was measured on human PBMCs. Briefly, hPBMCs were activated with anti-CD3 and anti-CD28. The masked cytokines, either cleaved with MMP or uncleaved, were titrated onto hPBMCs. Unmasked control and recombinant human IL-2 (rhIL-2) were used as controls. hPBMCs were stained for CD4+ T cells, CD8+ T cells, and pSTAT5. High EC50 values of pSTAT activity on CD8+ T cells by masked (uncleaved) cytokines indicate that the cytokines are effectively masked. The EC50 values by masked (uncleaved cytokines) of greater than 300-fold as compared to unmasked control indicates that the IL-2 cytokine is effectively masked.

TABLE 8
pSTAT on hPBMCs

| | Average | Average | Fold Masking | Fold Change |
|---|---|---|---|---|
| | EC50 (nM) | EC50 (nM) | (Uncleaved/ | (Cleaved/ |
| | without | with | Unmasked | Unmasked |
| Construct | MMP | MMP | Control) | Control) |
| rhIL-2 | 0.0017 | | | |
| Unmasked | 0.055 | | | 1 |
| Control | | | | |
| C5 | 411 | 0.25 | 7473 | 4.5 |
| C6 | 470 | 0.28 | 8546 | 5.1 |

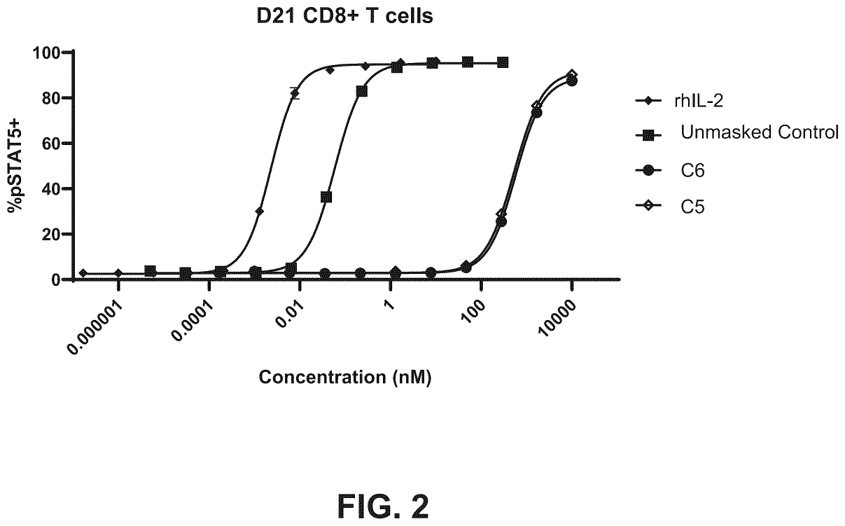

% of pSTAT in CD8+ T cells with different concentrations of rhIL-2, unmasked control, C5, and C6 are shown in FIG. 2. The average EC50 values for masked constructs on pSTAT activity of CD8+ T cell are shown in Table 8. Table 8 shows that the both C5 and C6 have EC50 values of greater than ˜7400 and ˜8500-fold as compared to the unmasked control, illustrating that the cytokines are effectively masked. Additionally, the IL-2 cytokine in the C5 and C6 constructs restored its activity when the masking moiety was cleaved by MMP, as illustrated by the low values of fold change as compared to unmasked control in Table 8.

### Example 2. Ex Vivo Tumor-Selective Cleavage of Masked IL-2 Cytokines

This example demonstrates the masked IL-2 cytokines of the present invention are activated by human tumors but not by human plasma.

Tumor and plasma samples from different cancer indications, melanoma, head and neck cancer (HNC), lung cancer, colon cancer, and renal cell carcinoma (RCC) were cultured and treated with constructs shown in Table 9. % of cleaved molecules were determined 24 hour post treatment. Preferentially, % of cleaved molecules in tumor is greater than 5% in at least one of the cancer indications tested, and % of cleaved molecules is below the lower limit of quantitation (LLOQ) in plasma.

TABLE 9
Exemplary Masked IL-2 Cytokines

| | | Masking | Targeting | Carrier | |
|---|---|---|---|---|---|
| Construct | Cytokine | Moiety | Moiety | Moiety | Linker |
| C5 | IL-2 V3 | N/A | Anti-PD1 | Fc Domain | Non-Cleavable |
| | N/A | VHH46 | Anti-PD1 | Fc Domain | Cleavable |
| C6 | IL-2 V3 | N/A | Anti-PD1 | Fc Domain | Non-Cleavable |
| | N/A | VHH1 | Anti-PD1 | Fc Domain | Cleavable |
| Non- | IL-2 V3 | N/A | Anti-PD1 | Fc Domain | Non-Cleavable |
| cleavable | | | | | |
| Control | N/A | VHH1 | Anti-PD1 | Fc Domain | Non-Cleavable |

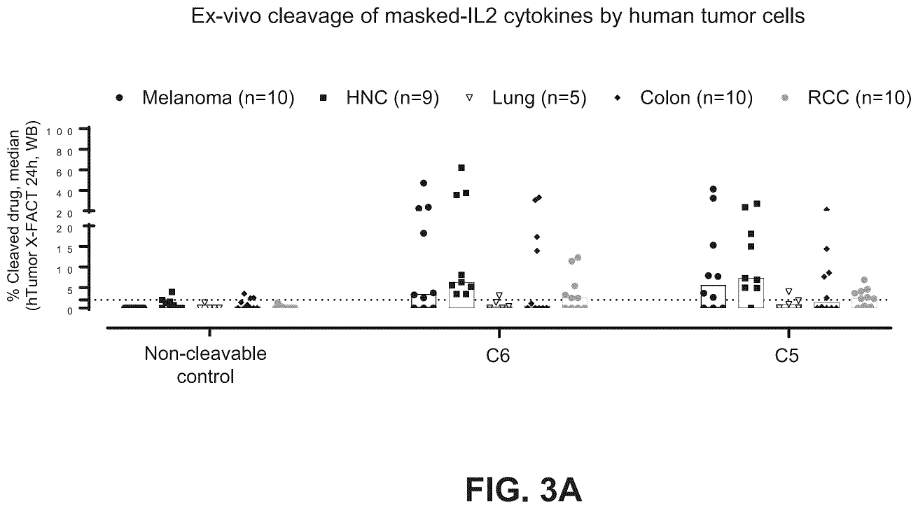

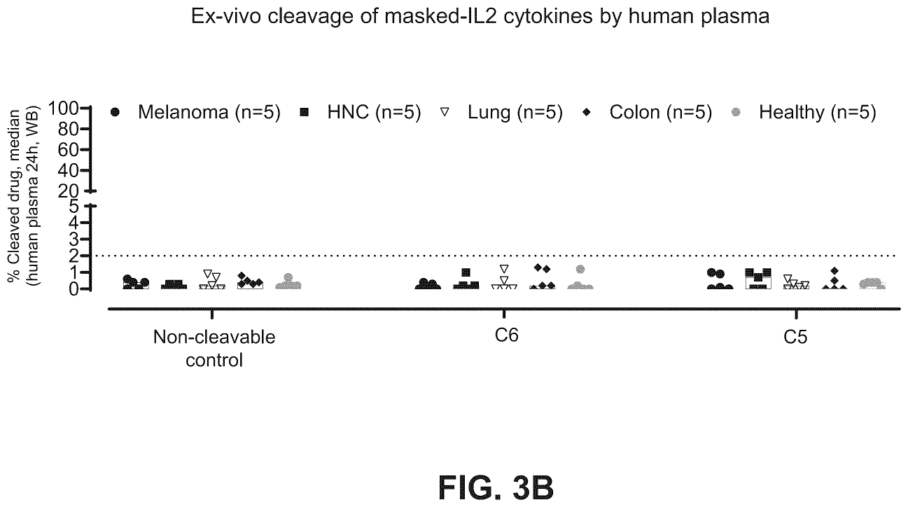

As shown in FIG. 3A, both C5 and C6 constructs were cleaved with significantly higher rate than the non-cleavable control, illustrating that C5 and C6 are activated in multiple tumor indications. Additionally, minimal cleavage of masked cytokines was observed in human plasma across all indications (FIG. 3B).

### Example 3. In Vivo Efficacy of Masked IL-2 Cytokines

This example demonstrates the masked IL-2 cytokines of the present invention are preferentially activated in tumor by tumor specific protease and reduce tumor volume in vivo. The masked cytokines tested contain three polypeptide chains (1 copy of chain A, 1 copy of chain B, and two copies of chain C) as illustrated in FIG. 1A and Table 10.

TABLE 10
Masked IL-2 Cytokines

| | Polypeptide | | SEQ ID |
|---|---|---|---|
| Construct | chain | Format | NO |
| C5 | A | [anti-PD-1 VH-CH1]-[Fc domain]- | 191 |
| | | [non-cleavable linker]- | |
| | | [attenuated IL-2] | |
| | B | [anti-PD-11 VH-CH1]-[Fc domain]- | 192 |
| | | [cleavable linker]- | |
| | | [VHH masking moiety] | |
| | C | Anti-PD1 VL-CL | 176 |
| C6 | A | [anti-PD-1 VH-CH1]-[Fc domain]- | 191 |
| | | [non-cleavable linker]- | |
| | | [attenuated IL-2] | |
| | B | [anti-PD-11 VH-CH1]-[Fc domain]- | 193 |
| | | [cleavable linker]- | |
| | | [VHH masking moiety] | |
| | C | Anti-PD1 VL-CL | 176 |
| Unmasked | A | [anti-PD-1 VH-CH1]-[Fc domain]- | |
| Control | | [non-cleavable linker]- | |
| | | [attenuated IL-2] | |
| | B | [anti-PD-11 VH-CH1]-[Fc domain] | |
| | C | Anti-PD1 VL-CL | |

### Masked IL-2 Cytokines are Tumor-Selective

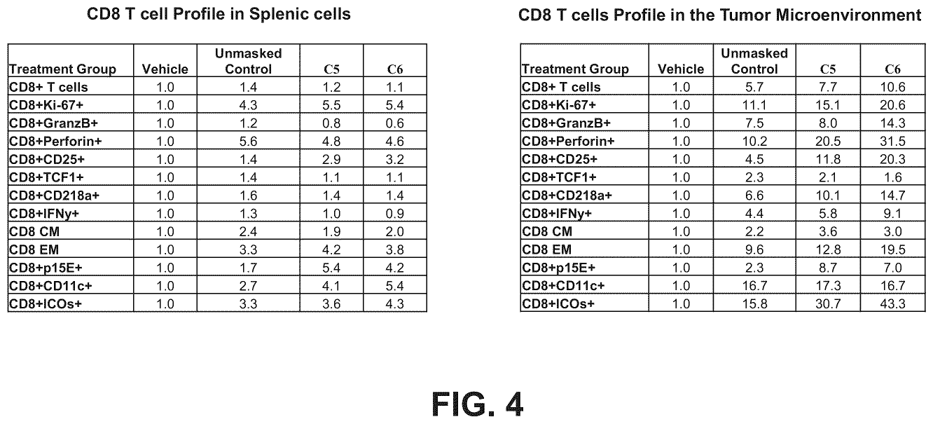

To determine whether the masked IL-2 cytokines of the present invention are preferentially activated in tumor, mice with hPD-1 (n=5 in each group) were subcutaneously injected with 0.5×10⁶ MC38 tumor cells. Treatment started when tumor volume reached ˜350-400 mm³. On day 0 and day 3, mice received construct C5, C6, unmasked control, or vehicle. On day 6, mice were sacrificed and FACS analysis was performed on blood, spleen, and tumor tissues. FIG. 4 shows CD8 T cell profiles in splenic cells and in the tumor microenvironment, respectively, illustrating that masked cytokines C5 and C6 demonstrate tissue-selective expansion of intra-tumoral CD8 T cells.

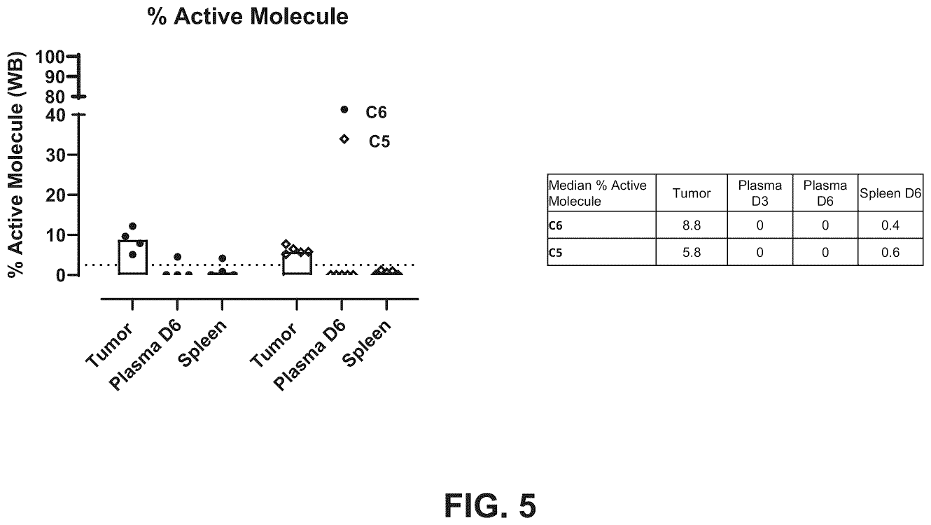

% of active IL-2 cytokines were also measured in tumor, plasma, and spleen. FIG. 5 shows that there is no detectable % active molecule in plasma and spleen for C5 and C6 treatment groups. Active IL-2 cytokines were detected in tumors from all C5 and C6 treatment groups, confirming that C5 and C6 are tumor selective with minimal activation in plasma or spleen.

### Masked IL-2 Cytokines are Efficacious In Vivo

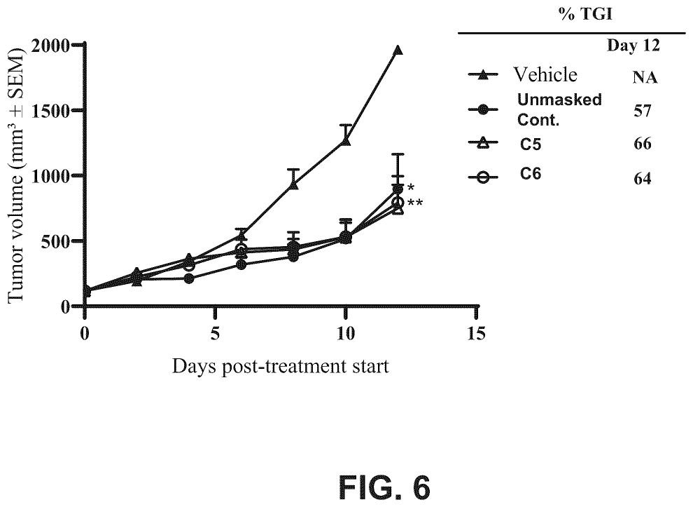

To determine in vivo efficacy of the masked IL-2 cytokines of the present invention, mice with hPD-1 (n=8 in each group) were subcutaneously injected with 0.5×10⁶ MC38 tumor cells in the right flank. Treatment started when tumor volume reached ˜100-150 mm³. On day 0 and day 5, mice were dosed with constructs C5, C6, unmasked control, or vehicle. Tumor volume was measured intermittently until day 12. FIG. 6 shows that both C5 and C6 were able to inhibit tumor growth significantly as compared to vehicle control. C5 and C6 had % of tumor growth inhibition of 66% and 64%, respectively.

Next, mice with hPD-1 (n=8 in each group) were subcutaneously injected with 1×10⁶ MB49 tumor cells in the right flank. On day 0 and day 5, mice were dosed with C6 construct, control masked IL-2 cytokine, control masked cytokine+anti-PD1 antibody, anti-PD1 antibody, or vehicle shown in Table 11. The control masked IL-2 cytokine used in this study comprises two polypeptides: (1) [Fc domain]-[non-cleavable linker]-[not-alpha IL-2]; and (2) [Fc domain]-[cleavable linker]-[IL-2 receptor β masking moiety];

TABLE 11
Treatment groups for in vivo study with MB49 tumor mice

| Treatment Group | Components | % TGI (day 12) |
|---|---|---|
| Monotherapy | C6 | 96 |
| Control Masked | Masked IL2 cytokine | 2 |
| IL-2 Cytokine | | |
| Combination | Control Masked IL2 cytokine + | 85 |
| Therapy | anti-PD1 antibody | |
| Antibody only | Anti-PD1 antibody | 50 |
| Neg. Control | Vehicle | N/A |

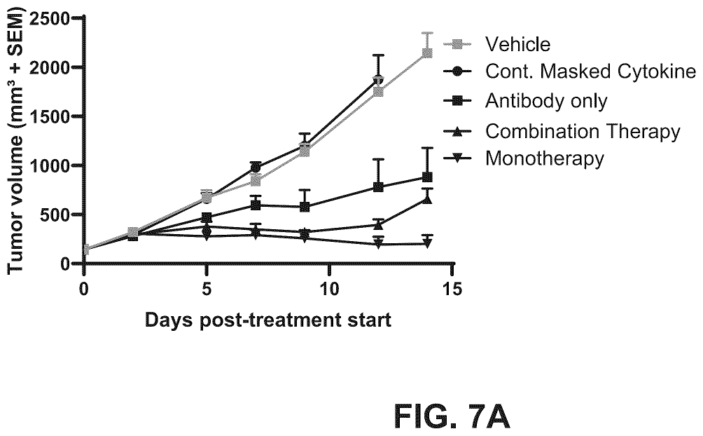

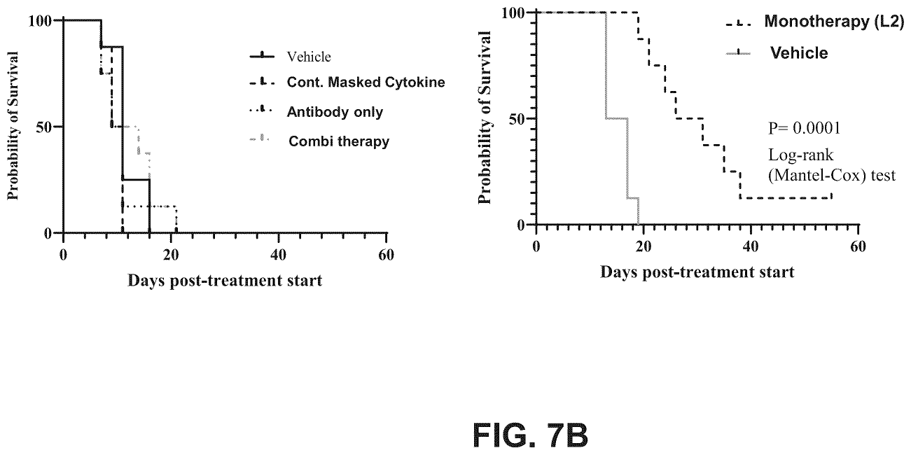

As shown in FIG. 7A, the masked IL-2 cytokine of the present invention inhibited tumor growth, illustrating significant monotherapy activity. Additionally, overall survival was significantly higher than any of other groups tested (FIG. 7B).

### Example 4. Exemplary Masked IL-2 Cytokines Block PD-1/PD-L1 Interaction

This example demonstrates that exemplary masked IL-2 cytokines maintain the ability to target PD-1 and block the PD-1/PD-L1 axis.

Exemplary masked IL-2 cytokines and a reference anti-PD1 antibody with blocking activity for PD-1 were tested with the Promega PD-1/PD-L1 Blockade Bioassay. The bioassay was set up and run according to the manufacturer's protocol.

Briefly, the day before performing the assay, cell recovery medium supplied with the bioassay kit was prepared by adding the supplied FBS (90% Ham's F-12/10% FBS). A vial of PD-L1 aAPC/CHO-K1 cells was gently thawed in the 37° C. water bath for 3 minutes. The thawed vial of PD-L1 aAPC/CHO-K1 cells was added to pre-warmed recovery medium, and the cell solution was mixed by inversion 2 times. A volume of 100 μL of cell suspension was added to a tissue-culture treated 96-well, white, flat-bottom assay plate. The plates were covered and incubated overnight (37° C., 5% CO₂).

On the following day, the assay plates containing the PD-L1 aAPC/CHO-K1 cells were removed from the incubator, and 95 μL of the recovery medium was removed from each of the wells. Serial dilutions of test articles, the exemplary masked IL-2 cytokines and reference antibody, were prepared in the provided assay buffer (99% RPMI 1640/1% FBS). 40 μL aliquots of the serial dilutions were added to the wells containing the PD-L1 aAPC/CHO-K1 cells with the final concentrations of the test articles ranging from 0.033-50 nM. Next, the PD-1 effector cells were prepared by thawing a vial of these cells in the 37° C. water bath for 3 minutes and adding the thawed effector cells to prewarmed assay buffer. The cell suspension was mixed by inversion 2 times before 40 μL of effector cell suspension was added to each of the wells. The assay plate was covered and incubated for 6 hours (37° C., 5% CO₂).

After the 6-hour incubation, the assay plates were removed from the incubator and let to equilibrate to ambient temperature for 5-10 minutes. 80 μL of Bio-Glo reagent was added to each well, and the plates were incubated for 5 minutes at ambient temperature prior to measuring the luminescence.

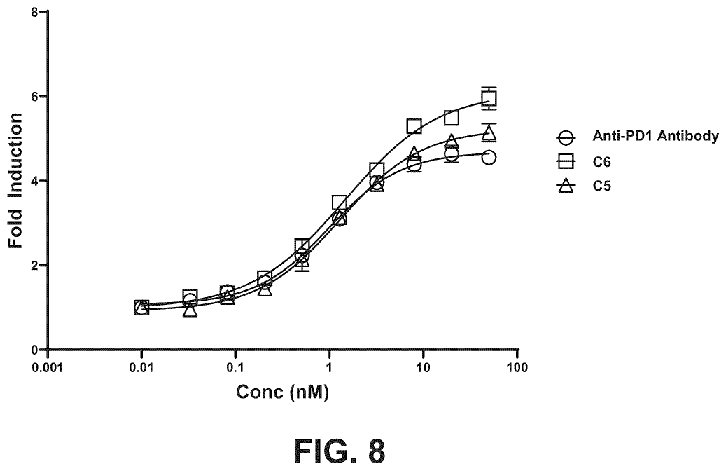

The addition of the exemplary masked IL-2 cytokine in this assay serves to block the interaction between PD-L1, from the PD-L1 aAPC/CHO-K1, and PD-1, from the PD-1 effector cells. By blocking the interaction, the exemplary masked IL-2 cytokine releases the inhibitory signal, resulting in T-cell receptor (TCR) signaling and an increase in luminescence. The fold induction of luminescence for exemplary masked IL-2 cytokines is shown in FIG. 8.

Data analysis was performed in GraphPad Prism. The fold induction was calculated by subtracting the luminescence of the background from the luminescence of the test article, and then dividing that by product of subtracting the background from a no antibody control (RLU [antibody−background]/RLU [no antibody control−background]). The calculated ECso value of each exemplary masked IL-2 cytokine, as shown in Table 12, was calculated using a nonlinear regression of the [Agonist] vs. response with a variable slope (four parameters).

TABLE 12
EC₅₀ Values for Exemplary Masked
IL-2 Cytokines

| Test Article | EC₅₀ |
|---|---|
| Anti-PD1 | 0.981 |
| Antibody | |
| C6 | 1.459 |
| C5 | 1.292 |

Overall, this example shows that exemplary masked IL-2 cytokines have similar ability in blocking the PD-1/PD-L1 axis as a reference anti-PD1 antibody with known blocking activity.

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

1. A masked cytokine comprising:
a) an attenuated interleukin 2 (IL-2) polypeptide,
b) a masking moiety comprising a heavy-chain-only antibody (VHH),
c) a carrier moiety, and
d) an anti-PD1 targeting moiety.

2. The masked cytokine of claim 1, wherein the attenuated IL-2 polypeptide comprises a mutation at amino acid position R38, F42, K43, Y45, E62, L72, and/or C125 compared to the sequence of a mature IL-2 having SEQ ID NO: 2.

3.-6. (canceled)

7. The masked cytokine of claim 1, wherein the VHH specifically binds to the IL-2 polypeptide.

8. The masked cytokine of claim 7, wherein the VHH comprises:
an HCDR1 comprising an amino acid sequence GSIFSINVMG (SEQ ID NO: 6),
an HCDR2 comprising an amino acid sequence AISSGGSTNYADSVKG (SEQ ID NO: 7), and
an HCDR3 comprising an amino acid sequence selected from ASSWYEDETDY (SEQ ID NO: 8).

9.-10. (canceled)

11. The masked cytokine of claim 7, wherein the VHH further comprises a C-terminal amino acid extension.

12.-16. (canceled)

17. The masked cytokine of claim 1, wherein the targeting moiety comprises an antigen binding domain selected from the group consisting of Fab, a single chain Fv (scFv), a single domain antibody (VHH), one or more CDRs, a variable heavy chain (VH), a variable light chain (VL), a Fab-like bispecific antibodies (bsFab), a single-domain antibody-linked Fab (s-Fab), an antibody, and a combination thereof.

18.-19. (canceled)

20. The masked cytokine of claim 1, wherein the targeting moiety comprises:

| | a heavy chain CDR1 sequence of |
|---|---|
| (SEQ ID NO: 172) | |
| | GYTFTNYY, |
| | a heavy chain CDR2 sequence of |
| (SEQ ID NO: 173) | |
| | INPSNGGT, |
| | a heavy chain CDR3 sequence of |
| (SEQ ID NO: 174) | |
| | ARRDYRFDMGFDY, |
| | a light chain CDR1 sequence of |
| (SEQ ID NO: 177) | |
| | KGVSTSGYSY, |
| | a light chain CDR2 sequence of |
| (SEQ ID NO: 178) | |
| | LAS, |
| | and |
| | a light chain CDR3 sequence of |
| (SEQ ID NO: 179) | |
| | QHSRDLPLT. |

21.-26. (canceled)

27. The masked cytokine of claim 1, wherein the carrier moiety comprises an Fc domain, and wherein the Fc domain comprises a first Fc polypeptide and second Fc polypeptide.

28. The masked cytokine of claim 27, wherein the first Fc polypeptide is linked to IL-2 polypeptide through a first linker and the second Fc polypeptide is linked to masking moiety through a second linker, and
wherein the first or the second linker comprises a tumor-associated protease cleavage site such that the masking moiety is released from the IL-2 polypeptide.

29.-37. (canceled)

38. The masked cytokine of claim 28, wherein the tumor-associated protease cleavage site comprises a sequence selected from Table 3.

39. (canceled)

40. A masked cytokine comprising:
a) an attenuated interleukin 2 (IL-2) polypeptide comprising an amino acid substitution of F42E and C125A;
b) a VHH masking moiety comprising a CDR1 of SEQ ID NO: 6, a CDR2 of SEQ ID NO: 7, and a CDR3 of SEQ ID NO: 8;
c) an Fc domain comprising a first Fc polypeptide linked to the attenuated IL-2 polypeptide through a first linker and a second Fc polypeptide linked to the VHH masking moiety through a second linker; and
d) an anti-PD1 targeting moiety.

41. The masked cytokine of claim 40, wherein:
a) the attenuated interleukin 2 (IL-2) polypeptide comprises SEQ ID NO: 4;
b) the VHH masking moiety comprises SEQ ID NO: 5 or SEQ ID NO: 12;
c) the Fc domain comprises the first Fc polypeptide of SEQ ID NO: 21 linked to the attenuated IL-2 polypeptide through the first linker and the second Fc polypeptide of SEQ ID NO: 20 linked to the VHH masking moiety through the second linker; and
d) the anti-PD1 targeting moiety comprises SEQ ID NOs: 169 and 175.

42. A masked cytokine comprising three polypeptides:
a) a first polypeptide comprising a variable heavy chain of an anti-PD1 targeting moiety, a first Fc polypeptide, a non-cleavable linker, and an attenuated IL-2 polypeptide;
b) a second polypeptide comprising a variable heavy chain of an anti-PD1 targeting moiety, a second Fc polypeptide, a cleavable linker, and a VHH masking moiety; and
c) a third polypeptide comprising a variable light chain of an anti-PD1 targeting moiety.

43.-52. (canceled)

53. The masked cytokine of claim 42, wherein:
a) the first polypeptide comprises:
the variable heavy chain of the anti-PD1 targeting moiety comprising SEQ ID NO: 169,
the first Fc polypeptide of SEQ ID NO: 21,
the non-cleavable linker, and
the attenuated IL-2 polypeptide of SEQ ID NO: 4;
b) the second polypeptide comprises:
the variable heavy chain of an anti-PD1 targeting moiety comprising SEQ ID NO: 169,
the second Fc polypeptide of SEQ ID NO: 20,
the cleavable linker comprising MPYDLYHP (SEQ ID NO: 34), and
the VHH masking moiety of SEQ ID NO: 5 or SEQ ID NO: 12, and
c) the third polypeptide comprises the variable light chain of the anti-PD1 targeting moiety comprising SEQ ID NO: 175.

54.-60. (canceled)

61. A nucleic acid encoding the masked cytokine of claim 1.

62. A vector comprising the nucleic acid of claim 61.

63. A host cell comprising the nucleic acid of claim 61.

64. A method producing a masked cytokine, comprising culturing the host cell of claim 63 under a condition that produces the masked cytokine.

65. A pharmaceutical composition comprising the masked cytokine of claim 1, and a pharmaceutically acceptable carrier.

66. A kit comprising the masked cytokine of claim 1.

67. A method of treating or preventing a neoplastic disease in a subject, the method comprising administering to the subject an effective amount of the masked cytokine of claim 1.

68. A method of treating or preventing an inflammatory or autoimmune disease in a subject, the method comprising administering to the subject an effective amount of the masked cytokine of claim 1.

69.-72. (canceled)
